(19) United States
(12) Patent Application Publication
(10) Pub. No.: US 2024/0424140 A1
(43) Pub. Date: Dec. 26, 2024

(54) METHODS AND COMPOSITIONS FOR DIRECT LINEAGE REPROGRAMMING OF ASTROCYTES TO OLIGODENDROCYTE LINEAGE CELLS

(71) Applicant: THE GOVERNING COUNCIL OF THE UNIVERSITY OF TORONTO, Toronto (CA)

(72) Inventors: Maryam FAIZ, Toronto (CA); Justine BAJOHR, Toronto (CA); Mershad SADRIA, Toronto (CA); Gary BADER, Toronto (CA)

(21) Appl. No.: 18/642,049

(22) Filed: Apr. 22, 2024

Related U.S. Application Data
(60) Provisional application No. 63/497,357, filed on Apr. 20, 2023.

(51) Int. Cl. A61K 48/00; A61K 38/17; C12N 15/86

(52) U.S. Cl. CPC A61K 48/0058; A61K 38/1709; C12N 15/86; C12N 2740/15043; C12N 2830/008

(57) ABSTRACT

The present application provides methods for production of induced oligodendrocyte lineage cells (IOLCs) from donor astrocytes comprising contact the donor astrocytes with a polynucleotide comprising a nucleic acid encoding a single transcription factor (TF), wherein the single TF is Olig2, Sox10, or Nkx6.2. Also provided are pharmaceutical compositions for performing the method and associated uses thereof.

### CROSS-REFERENCE TO RELATED APPLICATION

This application claims priority from U.S. patent application No. 63/497,357, filed Apr. 20, 2023, which is hereby incorporated herein in its entirety.

### REFERENCE TO A SEQUENCE LISTING SUBMITTED AS AN XML FILE

The Sequence Listing written in file PUS33107SEQLIST.xml is 51 kilboytes, was created on Apr. 22, 2024, and is hereby incorporated by reference.

### FIELD OF THE INVENTION

The present application pertains to the field of gene therapy. More particularly, the present application relates to gene therapy for direct lineage reprogramming of astrocytes, and vectors and compositions for use therein.

### INTRODUCTION

Direct lineage reprogramming (DLR) is a technology involving the conversion of one specialized cell type to another without the need for a pluripotent intermediate. To date, a wide variety of cell types have been successfully generated using direct reprogramming, both in vitro and in vivo. The newly converted cells have the potential to replace cells that are lost to disease and/or injury. Recently the use of DLR for in vivo central nervous system (CNS) tissue repair has been studied. The aim of this approach is to convert resident CNS cells into new target cells to replace those lost to disease or injury. While many studies have shown the production of different types of neurons via DLR from other neural cell types (1-3), few studies have examined DLR to oligodendrocytes (OLs).

OLs are mature, terminally differentiated cells that function, at least in part, to form myelin sheaths around axons. In the CNS, oligodendrocyte progenitor cells (OPCs) mature into OLs. OL lineage cells (OLCs) therefore include both OPCs and OLs. OLCs provide a broad range of functions, including trophic support of ensheathed axons, formation of myelin, ionic homeostasis, synaptic transmission, brain energy metabolism and learning and memory.

Prior studies looking at OL replacement via DLR have targeted fibroblasts or pericytes and used combinatorial transcription factor cocktails. In addition, to date these studies employed in vitro reprogramming, with the goal of transplanting OLs for cell replacement. However, in vivo DLR remains an interesting potential therapeutic route, since the loss or dysfunction of oligodendrocyte lineage cells (OLCs) is associated with many types of CNS disease and injury, including, multiple sclerosis, Alzheimer's disease, cerebral palsy, and spinal cord injury are all characterized by oligodendrocyte failure. Prior studies have also examined delivery of a single transcription factor to unspecified CNS cell types without any cell-specific promoter to direct expression and without targeting of astrocyte populations for DLR.

Different types of OLCs are lost in different types and stages of CNS disease or injury. For example, O4⁺ committed oligodendrocyte progenitor cells (COPs) are significantly more susceptible to infantile hypoxic-ischemic injuries than early oligodendrocyte progenitor cells (OPCs) (4), while mature OLs (mOLs) and myelin forming OLs (mfOLs) are primarily lost in progressive stages of Multiple Sclerosis (MS) (5). Considering the differences in OLC loss in different disease types and stages, there remains a need for DLR strategies to generate specific types of induced OLC (iOLC) cells, which could be matched to therapeutic need.

The above information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present application is to provide methods and compositions for direct lineage reprogramming of astrocytes to different stages and/or types of oligodendrocyte lineage cells. In accordance with an aspect of the present application, there is provided an in vivo method for production of induced oligodendrocyte lineage cells (iOLCs) from donor astrocytes in a subject in need thereof, the method comprising contacting the donor astrocytes with a polynucleotide comprising a nucleic acid encoding a single transcription factor (TF), wherein the single TF is Olig2, Sox10, or Nkx6.2, wherein the nucleic acid encoding the single TF is operably linked to an astrocyte-specific promoter sequence.

In some embodiments, the donor astrocytes are GFAP+cortical astrocytes.

In certain embodiments, the polynucleotide is within a vector, which is a viral vector, such as an adenoviral vector, an adeno-associated viral vector or a lentiviral vector, or a non-viral vector. Optionally, the polynucleotide, whether or not incorporated in a vector, is packaged in a nanoparticle, such as a lipid nanoparticle.

In certain embodiments of the method for production of iOLCs, the single TF is Olig2 and the iOLCs are induced oligodendrocyte progenitor cells. In other embodiments, the single TF is Sox10 and the iOLCs are myelinating olidodendrocytes and/or pre-myelinating oligodendrocyte precursor cells. In yet other embodiments, the single TF is is Nkx6.2 and the iOLCs are myelinating olidodendrocytes.

In accordance with another aspect, there is provided a vector comprising a nucleic acid encoding a single transcription factor (TF), wherein the single TF is Olig2, Sox10, or Nkx6.2, and wherein the nucleic acid encoding the single TF is operably linked to an astrocyte-specific promoter sequence. The vector a viral vector, such as an adenoviral vector, an adeno-associated viral vector or a lentiviral vector.

In accordance with another aspect, there is provided a viral particle and a host cell comprising the vector described above.

In accordance with another aspect, there is provided a pharmaceutical composition comprising a nucleic acid encoding a single transcription factor (TF), wherein the single TF is Olig2, Sox10, or Nkx6.2, and wherein the nucleic acid encoding the single TF is operably linked to an astrocyte-specific promoter sequence and a pharmaceutically acceptable carrier or excipient. In certain embodiments, the pharmaceutical composition is formulated for direct intracranial administration, intracranial injection, intracerebroventricular injection, intracisternal injection, intrathecal injection, intravenous injection, intraperitoneal injection, intraarterial, delivery via nanoparticles, and/or administration using focused ultrasound.

In some embodiments, the astrocyte-specific promoter sequence is a Nestin, Vimentin, Gfap, gfaABC1D (minimal GFAP promoter), Aldh1L1, S100b, ApoE, AldoC, C3, Lcn2, Serpina3a, Fabp5, Clu, S100a10, Aqp4, Slc1a3, Slca12, Slc14a1, Kcnj10, Fabp7, Fabp3, Col9a3, Aldoa, Tpi1, Acsbg1, Slc6a11, Atp1b2, Atb1a2, Acot11, Ppap2b, Slc38a3, Pygm, Scl25a18, Fads2, CAG, EF1a, CMV or CBA (chimeric CMV-chicken β-actin) promoter. In particular embodiments, the astrocyte-specific promoter sequence is a GFAP promoter.

In accordance with another aspect, there is provided a use of the vector, the viral particle, or the pharmaceutical composition as described herein for direct lineage reprogramming of astrocytes to induced oligodendrocyte lineage cells (iOLCs) in a subject in need thereof. In certain embodiments, this use is for prevention or treatment of a disease or disorder that affects astrocyte and/or oligodendrocyte lineage cells, or for treatment of an injury that affects astrocyte and/or oligodendrocyte lineage cells, or for prevention or treatment of a myelin-related disorder.

In accordance with another aspect, there is provided a method for prevention or treatment of a disease or disorder that affects astrocyte and/or oligodendrocyte lineage cells in a subject, said method comprising administering to the subject a nucleic acid for expression of a single transcription factor (TF), wherein the single TF is Olig2, Sox10, or Nkx6.2, in astrocytes within the subject. In certain embodiments, the disease or disorder is a myelin-related disease or disorder.

### BRIEF DESCRIPTION OF FIGURES

For a better understanding of the application as described herein, as well as other aspects and further features thereof, reference is made to the following description which is to be used in conjunction with the accompanying drawings, briefly described below.

FIG. 1 schematically depicts lentiviral vectors for expression of Olig2 (A), Sox10 (B) and Nkx6 (C) transcription factors and a control sequence (D).

FIG. 2 illustrates that Olig2, Sox10 and Nkx6.2 can reprogram astrocytes to oligodendrocyte lineage cells (A) Experimental design and timeline. (B) Quantification of tdTomato⁺ iOLCs. PDGFRattdTomato⁺ iOPCs, O4⁺tdTomato⁺ iCOPs and MBP⁺tdTomato⁺ imfOLs at 8 (n=5), 10 (n=4), 12 (n=5) and 14 (n=7) DPT (data are presented as mean±SEM, each data point represents one individual experiment; at each time point and for each cell type marker, a matched pairs one-way ANOVA or Kruskal-Wallis (d8, d10, d12) or one-way ANOVA (d14) was performed with Dunnet's post-testing (*=p<0.05, ***=p<0.001). (C) Representative images of PDGFRattdTomato⁺, O4⁺tdTomato⁺ and MBP⁺tdTomato⁺ cells. Single channel images are shown of the boxed cells (arrows indicate double positive cells).

FIG. 3 provides characterization of starting astrocyte type and cell division showing that the process of DLR is TF-specific (A) Number of donor cells with branched, flat or OL morphology at 6DPT (For each treatment a z-test for two population proportions was performed [Olig2-branched 1/9, flat 8/9, z=−3.30, p=0.0096; Sox10-branched 10/26, flat 16/26, z=−1.66, p=0.97; Nkx6.2-branched 4/6, flat 2/6, z=1.15; p=0.25; control-branch 0/5, flat 5/5, z=−3.16, p=0.0016], ***=p<0.001, ** p<0.01). (B) Representative images of tdTomato⁺astrocytes with a branched or flat morphology at 6DPT, and tdTomato⁺ cells with an iOLC morphology at 12 DPT (dotted circles indicate cell of interest). (C) Quantification of the number of cell divisions in tdTomato⁺ cells pre- and tdTomato⁺ iOLCs post-conversion. (D) Tracked single cells from Olig2-, Sox10-, Nkx6.2- and control (Cre) cultures from 6DPT (144 hrs) to 12DPT (284 hrs). Pre-conversion cell division is shown by white dots, post-conversion cell division is shown by black dots, conversion is shown by pink dots. B=branched, and F=flat astrocyte morphologies.

FIG. 4 illustrates scRNA-seq processing for initial UMAP generation. (A) Unique molecular identifier (UMI) per barcode plotted for each sample. Red dotted lines represent the threshold drawn, cells above the red line were kept. (B) The distribution of genes (nFeature), reads (nCount) and percent of mitochondrial reads (percent.mt) per sample. (C) UMAP prior to removal of microglia cluster (cluster 8). (D) UMAP of source cultures prior to removal of microglia cluster (cluster 8). (E) Expanded UMAP of soucr cultures ot visualize contribution of each culture to microglia cluster (cluster 8). (F) Top upregulated genes characterizaing each cluster observed in (C).

FIG. 5 provides annotated OLC gene expression in reprogrammed cultures. (A) Expression of genes common and unique to Olig2-, Sox10-, Nkx6.2- and control (Cre) UMAP clusters. (B) Violin plots of cell types annotated by Dugas et al. 2006 (34) within each cluster (EarlyD=early differentiation, LteD=late differentiation, OL=oligodendrocyte, OPC=oligodendrocyte progenitor cells). (C) Heat map of Dugas et al. (34) annoted gene set accrose cluster 0-8.

FIG. 6 illustrates molecular profiles of astrocyte to iOLC DLR at 14DPT are TF specific (A) UMAP of Olig2-, Sox10-, Nkx6.2- and control (Cre) transduced cells. (B) Heatmap of gene expression from Olig2-, Sox10-, Nkx6.2- and control (Cre) transduced cells. (C) UMAP of clustered Olig2-, Sox10-, Nkx6.2- and control (Cre) transduced cells. (D) Heatmap of top upregulated genes from each cluster in (C). (E) Proportion analysis of clusters found in Olig2-, Sox10-, Nkx6.2-, and control (Cre) transduced cells. (F) Violin plots of cell types annotated by Marques et al. 2016 within each cluster (OPC=oligodendrocyte progenitor cell, COP=committed oligodendrocyte progenitor, NfOL=newly forming oligodendrocyte, MfOL=myelin forming oligodendrocyte, mOL=mature oligodendrocyte). (G) Heat map of Marques et al. annotated gene set across clusters 0-8. (H) Overlay of astrocyte genes on UMAP plot. (I) Slingshot lineage analysis of clusters.

FIG. 7 illustrates astrocyte and lineage driving gene expression in reprogrammed cultures. (A) Heatmap of astrocyte gene expressing across Olig2-, Sox10-, Nkx6.2- and control (Cre) cultures. All=clusters found in groups i-iii, group iii=clusters specific to Nkx6.2- and Olig2-, Sox10-1. Sox10-2=clusters specific to Sox10 (group ii), Other=clusters from each group (i-iv). (B) Overlay of astrocyte genes on UMAP plot. Row I=genes found in groups i-iii, row ii=genes found in group iii (Nkx6.2 and Olig2 only), row iii=genes found in Sox10-1 cluster, row iv=genes found in sox10-2 cluster, row v=genes found in all four groups. (C) Random forest analysus of genes driving lineage trajectories.

FIG. 8 illustrates use of Sox10, Olig2 and Nkx6.2 convert GFAP+ cells to oligodendrocyte lineage cells. (A) Experimental design and timeline. (B) Quantification of PDGFRattdTomato⁺ iOPCs, O4⁺tdTomato⁺ iCOPs and MBP⁺tdTomato⁺ iOLs at 8 (n=5), 10 (n=4), 12 (n=5) and 14 (n=7) days post transduction (DPT). Data are presented as mean±SEM, each data point represents one individual cell culture experiment; at each time point and for each cell type marker, a matched pairs one-way ANOVA or Kruskal-Wallis (d8, d10, d12) or one-way ANOVA (d14) was performed with Dunnet's post testing (*=p<0.05, ***=p<0.001). (C) Representative images of PDGFRattdTomato⁺,O4⁺tdTomato⁺ and MBP⁺tdTomato⁺ cells at 12DPT. Single channel images are shown of the boxed cells (arrows indicate double positive cells). (D) UMAP clustering of Olig2-, Sox10-, Nkx6.2- and control (Cre) transduced cells at 14DPT (E) UMAP of (D) overlayed with treatment (Olig2-, Sox10-, Nkx6.2- and control (Cre)) (F) Proportion analysis of clusters found in Olig2-, Sox10-, Nkx6.2-, and control (Cre) transduced cells. (G) Heatmap of top upregulated genes from each cluster in (D).

FIG. 9 shows use of lineage tracing to confirm true conversion of astrocytes to oligodendrocyte lineage cells. (A) Experimental design, timeline and outcomes. (B) Quantification of tdTomato⁺zsGreen⁺PDGFRa⁺ iOPCs at 12DPT (n=3). Data are presented as mean±SEM, each data point represents one individual cell culture, a paired t-test was performed (**=p<0.01). (C) Quantification of tdTomato⁺zsGreen⁺O4⁺ iCOPs at 14DPT (n=4). Data are presented as mean±SEM, each data point represents one individual cell culture, a Wilcoxon test was performed (ns). (D) Quantification of tdTomato⁺zsGreen⁺MBP⁺ iOLs at 12DPT (n=4 for Sox10, Nkx6.2, n=3 for Cre). Data are presented as mean±SEM, each data point represents one individual cell culture, a one-way ANOVA with Dunnet's post testing was performed (*=p<0.05). (E) Representative image of PDGFRattdTomato⁺zsGreen⁺ cells 12DPT. Single channel images are shown of the boxed cells (arrows indicate triple positive cells, scale bar=50 um (merge) and 20 um (single channel)). (F) Representative image of MBP⁺tdTomato⁺zsGreen⁺ cells 12DPT. Single channel images are shown of the boxed cells (arrows indicate triple positive cells, scale bar=50 um (merge) and 20 um (single channel)). (G) Representative tdTomato⁺zsGreen⁺ cell with astrocyte-like morphology at onset (7DPT) and OLC expression at the end (12DPT) of live cell tracking (arrow indicates tracked cell, scale bar=50 um). (H) Representative tdTomato^{neg}zsGreen⁺ cell with astrocyte-like morphology at onset (7DPT) and OLC expression at the end (12DPT) of live cell tracking (arrow indicates tracked cell, scale bar=50 um).

FIG. 10 shows 14DPT scRNA-seq processing for initial UMAP generation. (A) Distribution of genes (nFeature), reads (nCount) and percent of mitochondrial reads (percent.mt) per sample. (B) UMAP clustering prior to removal of microglia cluster. (C) Top upregulated genes characterizing each cluster observed in (B).

FIG. 11 depicts DLR timecourse scRNA-seq processing for initial UMAP generation. (A) Distribution of genes (nFeature), reads (nCount) and percent of mitochondrial reads (percent.mt) per sample. (B) UMAP clustering prior to removal of microglia and VLMC clusters. (C) Top upregulated genes characterizing each cluster observed in (B).

FIG. 12 depicts characterization of DLR cultures. (A) Quantification of contaminating iOLCs in post-natal astrocyte cultures 3DPT. (B) Quantification of MBP expression in tdTomato+ transduced cells 22DPT (n=3, data are presented as mean±SEM, each data point represents one individual cell culture, a matched pairs one way ANOVA was performed (ns)).

FIG. 13 shows a comparison of DLR clusters 14DPT to established datasets of OLCs. (A) Violin plots of cell types annotated by Marques et al. 2016 within each cluster (OPC=oligodendrocyte progenitor cells, COPs=committed oligodendrocyte progenitors, NFOL=newly formed oligodendrocyte, MFOL=myelin forming oligodendrocyte, mOL=mature oligodendrocyte). (B) Violin plots of cell types annotated by Dugas et al. 2006 within each cluster (OL=oligodendrocyte, OPC=oligodendrocyte progenitor cells).

FIG. 14 shows (A) Quantification of (i) tdTomato⁺ and (ii) zsGreen⁺PDGFRa⁺ iOPCs at 12DPT (n=3). Data are presented as mean±SEM, each data point represents one individual cell culture, a paired t-test for (i) and a Wilcoxon test for (ii) was performed (**=p<0.01). (B) Quantification of (i) tdTomato⁺ and (ii) zsGreen⁺O4⁺ iCOPs at 14DPT (n=4). Data are presented as mean±SEM, each data point represents one individual cell culture, a paired t-test was used. (C) Quantification of (i) tdTomato⁺ (ii) zsGreen⁺MBP⁺OLs at 12DPT (n=3). Data are presented as mean±SEM, each data point represents one individual cell culture, a matched pairs one way ANOVA with Geisser-Greenhouse correction and Dunnet's post testing was used (*=p<0.05).

FIG. 15 shows characterization of DLR using scRNA-seq shows terminal oligodendrocyte cluster of cells at day 14 driven by Sox10. (A) UMAP clustering of Sox10 and Cre control treated cells from prior to transduction, as well as 3, 8 and 14DPT (B) Canonical astrocyte gene expression (log-normalized, y-axis) separated by cluster (x-axis) and coloured by timepoint and treatment group. (C) Canonical gene expression (log-normalized, y-axis) of NG2 glia, VLMC, OLC, microglia and proliferation, separated by cluster (x-axis) and coloured by timepoint and treatment group. (D) UMAP clustering from (A) overlayed with timepoint and treatment group (E) Slingshot lineage analysis of Sox10 and Cre control treated clusters overlaid with UMAP embeddings. (F) Monocle3 lineage analysis of Sox10 and Cre control treated clusters overlaid on UMAP plot and coloured by pseudotime predictions. (G) The number of cells (y-axis) per cluster (x-axis) originating from each of the coloured timepoint⁺ treatment groups. (H) CellOracle modeling of in silico Sox10 knock out (KO) overlaid onto UMAP plot. Arrows indicate trajectory prediction with Sox10 KO. (I) Cell Oracle modeling of in silico Sox10 knock in (KI) overlaid onto UMAP plot. Arrows indicate trajectory prediction with Sox10 KI (J) Clustered, top differentially expressed genes (dots, y-axis) between 14DPT Sox10 (D14_S, Beige, left) and Cre (D14_C, Blue, right) control treated cells from cluster 6. Size of dot is scaled to percent of cells in the cluster expressing that gene, colour of dot represents the average scaled expression of the gene across cells. (K) Predicted cell types following Fatecode perturbation on node 16 of the latent layer in the Sox10 and control treated dataset. Blue represents the number of cells prior to perturbation. Orange (overlayed) represents the number of cells following perturbation.

FIG. 16 illustrates differential gene expression of clusters in the Monocle3 trajectory. (A) Genes enriched in leaf 14 of Monocle3 trajectory. Cells in UMAP colored according to log-normalized gene expression values. (B) Genes enriched in leaf 11 of Monocle3 trajectory. Cells in UMAP colored according to log-normalized gene expression values.

### DETAILED DESCRIPTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The terms “comprise”, “comprising”, “include”, “including”, “have” and “having” as used herein will be understood to mean that the list following is non-exhaustive and may or may not include any other additional suitable items, for example one or more further feature(s), component(s) and/or ingredient(s) as appropriate. The terms “such as”, “for example” and “e.g.” as used herein are non-limiting and are for illustrative purposes only. “Including” and “including but not limited to” are used interchangeably.

Reference throughout this specification to “one embodiment,” “an embodiment,” “another embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” or “approximately” refers to a quantity, level, value, number, frequency, percentage, dimension, size, amount, weight or length that varies by as much as 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±10%, ±9%, ±8%, ±7%, ±6%, ±5%, ±4%, ±3%, ±2%, or ±1% about a reference quantity, level, value, number, frequency, percentage, dimension, size, amount, weight or length.

As used herein, the term “effective amount” of a substance is an amount sufficient to produce a desired effect. In some embodiments, an effective amount of a nucleic acid or vector is an amount sufficient to infect a sufficient number of target cells of a target tissue of a subject. The effective amount will depend on a variety of factors such as, for example, the species, age, weight, health of the subject, and the mode or site of administration, and may thus vary among subjects and administrations.

As used herein, the terms “peptide”, “polypeptide” and “protein” are used interchangeably and thus the term polypeptide may be used to refer to a full-length protein and may also be used to refer to a fragment of a full-length protein, and/or functional variants thereof. The terms also apply to amino acid polymers in which one or more amino acids are chemical analogues or modified derivatives of a corresponding naturally-occurring amino acids.

As used herein, the terms “polynucleotide”, “nucleotide”, and “nucleic acid” may be used interchangeably and may refer to a deoxyribonucleotide or ribonucleotide polymer, in linear or circular conformation, and in either single- or double-stranded form, including but not limited to: RNA, DNA, mRNA, cDNA, etc. These terms may be used to reference full length sequences, functional variants, and/or fragments thereof. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The term “treating” or “treatment” is art-recognized and includes inhibiting a disease, disorder or condition in a subject, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected. The term “treating” or “treatment” is also used to refer to amelioration of an injury or at least one symptom thereof. As used herein, the term “amelioration” means the prevention, reduction or palliation of a state, or improvement of the state of a subject. Amelioration includes, but does not require, complete recovery or complete prevention of a disease, disorder, condition or injury.

The term “preventing” is art-recognized and includes stopping a disease, disorder or condition from occurring in a subject, which may be predisposed to the disease, disorder and/or condition but has not yet been diagnosed as having it. Preventing a condition related to a disease includes stopping the condition from occurring after the disease has been diagnosed but before the condition has been diagnosed.

As used herein, the terms “improve,” “increase” or “reduce,” or grammatical equivalents, indicate values that are relative to a reference (e.g., baseline) measurement, such as a measurement taken under comparable conditions (e.g., in the same individual prior to initiation of a treatment described herein, or a measurement in a control individual (or multiple control individuals) in the absence of treatment) described herein.

As used herein, the terms “target site” or “target tissue” refers to any cell, tissue, or region in a subject that is affected by a disease, disorder, condition or injury to be treated. In some embodiments, target cells, target tissues, or target regions include those cells, tissues, or regions in which there is a loss or dysfunction of oligodendrocyte lineage cells. In some embodiments, target cells, target tissues, or target regions include those cells, tissues, or regions that display a disease-associated pathology, symptom, or feature.

The term “pharmaceutical composition” refers to a formulation containing the disclosed compounds or materials in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active component (e.g., a polynucleotide or vector) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “vector” is meant to refer to a vehicle to artificially carry foreign genetic material into a host cell, such as a recombinant plasmid or virus that comprises a nucleic acid to be delivered into the host cell, either In vitro or in vivo.

As used herein, the term “expression vector” refers to a vector that directs expression of an RNA or polypeptide from sequences linked to transcriptional regulatory sequences on the vector. The sequences expressed will often, but not necessarily, be heterologous to the host cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and, as appropriate, secreting proteins, including where applicable, but not limited to, for example, transcription, transcript processing, translation and protein folding, modification and processing. Expression products include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may be a mutant gene and may or may not include regions preceding and following the coding region, e.g., 5′ untranslated (5′UTR) and Kozak sequence, or “leader” sequences and 3′ UTR or “trailer” sequences, as well as intervening sequences (introns) between individual coding segments (exons).

Typically, direct lineage reprogramming (DLR) has been achieved by the ectopic expression of transcription factors (TFs). Seminal work in the Tesar Lab demonstrated the combined power of oligodendrocyte lineage determinants, Olig2, Sox10 and Nkx6.2, in inducing OLC fate (6). Olig2/Sox10/Nkx6.2 was shown to reprogram fibroblasts to induced OPCs (iOPCs), and more recently, the combination of Sox10 and Olig2 was used convert pericytes to iOPCs (7). Of interest, each of these “Tesar factors”, Olig2, Sox10, and Nkx6.2, play a different role and show different temporal expression during OL development. Olig2, long considered an OL lineage fate determinant (8), is also strongly expressed in immature astrocytes, which suggests a broad role in early glial commitment (9, 10). Sox10 is important throughout OL development; Sox10 promotes early OL lineage specification by re-inducing Olig2 (11) and by inhibiting Sufu (12), but is also required for OL survival following myelination (13). In contrast, Nkx6.2 is expressed late in OL development, with myelin genes Mbp and Mog, and plays a role in regulating myelination (14, 15).

The present inventors have now demonstrated that by taking advantage of the unique roles of each of these TFs in development, these single factors can be harnessed to push the creation of distinct stage and/or types of induced iOLCs.

As DLR in the CNS requires the removal of a resident cell to generate a target cell, it is important to also consider how donor cell type influences the specific types of target OLCs produced. Astrocytes are an attractive donor cell type given their shared neural origin with oligodendrocytes (16, 17). Astrocytes may already have relevant epigenetic marks and active transcription factors that could make DLR faster or more efficient (18, 19). In addition, closely related cells may require fewer TFs for conversion. Indeed, conversion of astrocytes to an iOLC-like cell has been suggested using only Sox10 (20). However, astrocytes are molecularly, and functionally heterogeneous, and have been demonstrated to have regional differences in DLR capability (21). Therefore, it was important to take into consideration how astrocyte diversity influences DLR driven by different TFs.

The present inventors have now surprisingly found that delivery of a single TF, Olig2, Sox10, or Nkx6.2, can convert astrocytes from various target sites or tissues to different types of iOLCs, depending on both the target astrocyte population and the TF. With live cell imaging, and single cell RNA sequencing (scRNA-seq) the inventors have demonstrated that each of these single TF-based DLR strategies converts different types of astrocytes, and follows different paths to iOLC generation. Altogether, these results provide a method that facilitates DLR of different donor astrocyte cell types based on choice of transcription factor to produce specific types of target oligodendrocyte lineage cells.

The therapeutic method and use of the present application comprises introduction of a single TF, Olig2, Sox10, or Nkx6.2, or nucleic acid encoding the single TF, to the site or tissue where intervention is required. The selection of TF and target astrocyte population is used to tailor the treatment to the disease, disorder or injury to be treated. The present method or use facilitates expression of the single TF-encoding nucleic acid in a specific region that has been injured or that has been affected by disease, such that local astrocytes are converted to the type of iOLC useful for treatment of the disease, disorder or injury, or symptoms thereof. Consequently, in the present method or use, a specific population of astrocytes can be targeted and/or leveraged to provide a therapeutic benefit that is tailored to a particular disease, disorder or injury.

### Nucleic Acids and Vectors

The methods, uses and compositions of the present application employ nucleic acids encoding a single transcription factor (TF), wherein the single TF is Olig2, Sox10, or Nkx6.2, and wherein the coding sequence for the single TF is under control of an astrocyte-specific promoter sequence. According to the present disclosure, such nucleic acids are useful in DLR of astrocytes to OLCs. In some embodiments, such nucleic acids have or comprise nucleic acid sequences as set forth in SEQ ID NO:1 (encoding Olig2), SEQ ID NO:2 (encoding Sox10) or SEQ ID NO: 3 (encoding Nkx6.2). In some embodiments, useful nucleic acids have at least 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% overall sequence identity with one or more of SEQ ID NO:1, 2 or 3 and encode functional variants of Olig2, Sox10, or Nkx6.2, respectively. A functional variant of a nucleic acid of SEQ ID NO:1, 2 or 3 is one that encodes a TF that retains the ability to be useful in DLR of astrocytes to OLCs.

A variety of methods of making nucleic acids that are “variants” with respect to a reference nucleic acid (e.g., a naturally-occurring or other reference nucleic acid) are well known in the art. These include, for example, procedures in which nucleic acid sequences obtained from natural isolates are modified to generate nucleic acids that encode polypeptides having characteristics that enhance their value, for example by enhancing their expression in a particular target cell, tissue or site.

The present application further provides a nucleic acid, or nucleic acid construct, designed to express an Olig2, Sox10, or Nkx6.2 transcription factor polypeptide in a target cell, tissue or site. Such a nucleic acid comprises a polynucleotide encoding an Olig2, Sox10, or Nkx6.2 transcription factor as described herein operably linked to one or more control sequences that direct the expression of the coding sequence in the target cell, tissue or site under conditions compatible with the control sequences. In some embodiments, the nucleic acid is designed for expression in target astrocytes.

In some embodiments, the nucleic acid is DNA, while in other embodiments, the nucleic acid is mRNA.

In addition to the polynucleotide encoding a transcription factor polypeptide, the nucleic acid can contain one or more regulatory elements operably linked to the coding sequence. As used herein, “operably linked” refers to positioning of a regulatory element in a vector relative to a nucleic acid in such a way as to permit or facilitate expression of the encoded polypeptide. Examples of regulatory elements can include, without limitation, translation initiation sequences (e.g., a Kozak consensus sequence or a tissue-specific Kozak sequence (e.g., McClements et al. Mol. Vis. 2021, 27:233-242) to avoid expression in non-target tissues), promoter sequences, enhancer sequences, response elements, signal peptides, internal ribosome entry sequences, 5′UTR, 3′UTR, polyadenylation signals, terminators, or inducible elements that modulate expression (e.g., transcription or translation) of a nucleic acid.

For example, a promoter can be included in the nucleic acid to facilitate transcription of the nucleic acid encoding a transcription factor polypeptide. A promoter can be constitutive or inducible, and can affect the expression of a nucleic acid encoding a polypeptide in a general or tissue-specific manner. Examples of cell-specific and/or tissue-specific promoters that can be used to drive expression of a transcription factor polypeptide in astrocytes include, without limitation, Nestin, Vimentin, Gfap, gfaABC1D (minimal GFAP promoter), Aldh1L1, S100b, ApoE, AldoC, C3, Lcn2, Serpina3a, Fabp5, Clu, S100a10, Aqp4, Slc1a3, Slca12, Slc14a1, Kcnj10, Fabp7, Fabp3, Col9a3, Aldoa, Tpi1, Acsbg1, Slc6a11, Atp1b2, Atb1a2, Acot11, Ppap2b, Slc38a3, Pygm, Scl25a18, Fads2, CAG, EF1a, CMV and CBA (chimeric CMV-chicken β-actin) promoters. In particular embodiments, a GFAP promoter can be included in a viral vector to facilitate transcription of a nucleic acid encoding a transcription factor polypeptide in astrocytes.

In some embodiments, a single TF-encoding nucleic acid is inserted into a viral vector for delivery to a subject. For example, retrovirus vectors can be used as a recombinant delivery system for transferring single TF-encoding nucleic acid for expression in vivo. Retroviruses useful in methods of the present disclosure include, but are not limited to, murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), FBR marine osteosarcoma virus (FBR MSV), Moloney murine sarcoma virus (Mo-MSV), Abelson murine leukemia virus (A-MLV), Avian myelocytomatosis virus-29 (MC29), Avian erythroblastosis virus (AEV) and all other retroviridiae including lentiviruses. A replication defective retrovirus can be packaged into virions that can be used to infect a target cell through the use of a helper virus by standard techniques.

In accordance with specific embodiments of the present disclosure there is provided a lentiviral vector comprising a single TF-encoding nucleic acid as described herein.

In other embodiments, adenovirus-derived vectors are used to deliver a single TF-encoding nucleic acid. The genome of an adenovirus can be manipulated such that it encodes and expresses the TF, but is inactivated in terms of its ability to replicate in a normal lytic viral life cycle.

In some embodiments, an adeno-associated virus (AAV) is used to deliver a single TF-encoding nucleic acid. AAV vectors have been successfully used to introduce a variety of nucleic acids into different cell types, such that these vectors and method for their manufacture are now well known to those of skill in the art. Useful AAVs include those that normally infect humans (e.g., serotypes 1, 2, 3A, 3B, 4, 5, and 6) or primates (e.g., serotypes 1 and 4). In addition, serotypes AAV9 and AAVrh10 can be particularly useful since they can transport across the blood-brain barrier and can, therefore, be useful as a non-invasive method to target the CNS.

In other embodiments, non-viral methods are useful to deliver a single TF-encoding nucleic acid to a subject. Such non-viral methods of gene transfer can exploit mechanisms normally used by mammalian cells for uptake and intracellular transport of macromolecules. For example, liposomal delivery systems, poly-lysine conjugates, and artificial viral envelopes can be used. In some embodiments, a single TF-encoding nucleic acid is entrapped in liposomes bearing positive charges on their surface (e.g., lipofectins). In some embodiments, a liposome can be conjugated to a targeting agent. In some embodiments in which the single TF-encoding nucleic acid is used in a liposomal delivery system, the nucleic acid in mRNA.

Certain cationic polymers (“complexation agents”) known to spontaneously bind to and condense nucleic acids into nanoparticles can also be used including, e.g., naturally occurring proteins, peptides, or derivatives, as well as synthetic cationic polymers such as polyethylenimine (PEI), polylysine (PLL), etc. Many useful polymers contain both chargeable amino groups, to allow for ionic interaction with negatively charged DNA phosphate, and a degradable region, such as a hydrolyzable ester linkage. Examples of these include, without limitation, poly(alpha-(4-aminobutyl)-L-glycolic acid), network poly(amino ester), and poly(beta-amino esters). Such complexation agents can protect DNA against degradation, e.g., by nucleases, serum, components, etc., and create a less negative surface charge, which may facilitate passage through hydrophobic membranes (e.g., cytoplasmic, lysosomal, endosomal, nuclear) of the cell. Certain complexation agents facilitate intracellular trafficking events such as endosomal escape, cytoplasmic transport, and nuclear entry, and can dissociate from the nucleic acid.

In some embodiments, cationic polymers are used together with cyclodextrin as a non-viral delivery vector for the single TF-encoding nucleic acids described herein.

### Therapeutic Use and Compositions

The present application provides a method and use for direct lineage reprogramming of astrocytes to oligodendrocytes for the treatment of a disease, disorder or injury that affects astrocyte and/or oligodendrocyte lineage cells. According to the present method or use, a specific population of astrocytes can be targeted and/or leveraged to provide a therapeutic benefit that is tailored to treatment of a particular disease, disorder or injury and/or a particular symptom thereof. By targeting specific types or populations of astrocytes according to the method of the present disclosure, off target effects are minimized both within the broad astrocyte population and among the neural cell population (i.e., neurons and oligodendrocytes). Targeting of particular astrocyte populations can be achieved, for example, through the use of a specific AAV serotype or specific lipid nanoparticles (LNPs) that can target astrocytes, which can be used alone or in combination with a specific promoter in the nucleic acid for expression in the target astrocyte population.

As described herein, a mammal can be treated by delivering a nucleic acid encoding one of Olig2, Sox10, or Nkx6.2 transcription factor to astrocytes within the mammal's CNS, in a manner that triggers the astocytes at the target site to form OLCs.

In a non-limiting embodiment, the present method or use can be used in treatment of MS. In one example of this embodiment, a single TF-encoding nucleic acid is administered or directed to regions of the optic nerve where significant pathology is found.

In another non-limiting embodiment, the present method or use can be used in treatment of spinal cord injury by administration or direction of a single TF-encoding nucleic acid to the site of injury in the cord.

In another non-limiting embodiment, the present method or use can be used in treatment of Alzheimer's disease.

In another non-limiting embodiment, the present method or use can be used in treatment of a non-traumatic brain injury, such as cortical stroke, or traumatic brain injury, whereby a single TF-encoding nucleic acid is administered or directed to the site of injury, e.g., the cortex in the case of a cortical stroke.

In another non-limiting embodiment, the present method or use can be used in treatment of perivascular leukomacia or cerebral palsy.

Depending on the target tissue or site, expression of the single TF-encoding nucleic acid and subsequent conversion of local astrocytes to iOLCs, can: provide increased myelination; change inflammatory environment/immune response; decrease axonal degeneration; to provide better functional outcomes (motor, cognitive, vision). Accordingly, in another non-limiting embodiment, the present method or use can be used in the treatment of diseases or disorders that: affect or impair myelin production or function; are characterized by impaired inflammatory or immune response; and/or are characterized by axonal degeneration.

In specific embodiments the formed OLCs function to enhance myelin production in the mammal. In some embodiments in which the formed OLCs function to enhance myelin production, the disorder to be treated is a myelin-related disorder, which is optionally multiple sclerosis (MS), Alexander Disease (AxD), neuromyelitis optica (NMO), transverse myelitis, chronic inflammatory demyelinating polyneuropathy, Guillain-Barre Syndrome, progressive multifocal leukoencephalopathy (PML), encephalomyelitis (EPL), central pontine myelolysis (CPM), adrenoleukodystrophy, Alexander's disease, Pelizaeus Merzbacher disease (PMD), Wallerian Degeneration, optic neuritis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, AR, Bassen-Komzweig syndrome, Marchiafava-Bignami syndrome, metachromatic leukodystrophy, trigeminal neuralgia, acute disseminated encephalitis, Marie-Charcot-Tooth disease or Bell's palsy.

In other embodiments, the formed OLCs function to change an inflammatory environment or immune response, to decrease axonal degeneration and/or to provide better functional outcomes, for example, by providing improved motor activity, improved cognition, improved sensory activity and/or improved vision.

In a specific embodiment, Nkx6.2-expression has been found by the present inventors to direct an astrocyte population with gene expression enriched in Aldoc, Slc1a2, Gjb6, Mertk and Fam107a towards a myelinating oligodendrocyte. Accordingly, in some non-limiting embodiments, there is provided a method comprising use of an Nkx6.2-expressing nucleic acid to reduce an astrocyte population having gene expression enriched in Aldoc, Slc1a2, Gjb6, Mertk and Fam107a and/or to provide DLR of an astrocyte population having gene expression enriched in Aldoc, Slc1a2, Gjb6, Mertk and Fam107a to produce myelinating oligodendrocytes.

In a specific embodiment, Olig2-expression has been found by the present inventors to direct an astrocyte population with gene expression enriched in Aldoc, Slc1a2, Gjb6, Mertk and Fam107a towards an oligodendrocyte progenitor cell. Accordingly, in some non-limiting embodiments, there is provided a method comprising use of an Olig2-expressing nucleic acid to reduce an astrocyte population with gene expression enriched in Aldoc, Slc1a2, Gjb6, Mertk and Fam107a and/or to provide DLR of an astrocyte population with gene expression enriched in Aldoc, Slc1a2, Gjb6, Mertk and Fam107a to produce oligodendrocyte progenitor cells.

In another specific embodiment, Sox10-expression has been found by the present inventors to provide direct lineage reprogramming of two populations of astrocytes with distinct gene expression profiles (astrocytes enriched in Nes, Id3, Vim, Tspo and Cryab and astrocytes enriched in C3, Lcn2, Thbs1, Tnc and Serpina3n). These distinct astrocyte populations generate different oligodendrocyte cell types, either myelinating oligodendrocytes or committed oligodendrocyte progenitors. Accordingly, in some non-limiting embodiments, there is provided a method comprising use of a Sox10-expressing nucleic acid to reduce an astrocyte population with gene expression enriched in Nes, Id3, Vim, Tspo and Cryab and/or to provide DLR of an astrocyte population with gene expression enriched in Nes, Id3, Vim,
Tspo and Cryab to produce myelinating oligodendrocytes. In other non-limiting embodiments, there is provided a method comprising use of a Sox10-expressing nucleic acid to reduce an astrocyte population with gene expression enriched in C3, Lcn2, Thbs1,
Tnc and Serpina3n and/or to provide DLR of an astrocyte population with gene expression enriched in C3, Lcn2, Thbs1, Tnc and Serpina3n to produce committed oligodendrocyte progenitors.

Also provided herein are compositions comprising the TF-encoding nucleic acids as described above and one or more diluent, excipient or carrier, which is one or more pharmaceutically acceptable diluent, excipient or carrier when the composition is formulated for administration to treat a subject.

In one embodiment, the TF-encoding nucleic acid of the present disclosure can be delivered directly within an expression vector designed for expression of the coding sequence within the astrocytes.

In other embodiments, the TF-encoding nucleic acid can be packaged within a particle (especially, a nanoparticle), such as a liposome (e.g., a liposome nanoparticle), for delivery to astrocytes in the target tissue or site. In one example of this embodiment, the TF-encoding nucleic acid is mRNA.

In accordance with another aspect, there is provided a pharmaceutical composition comprising the single TF-encoding nucleic acid and a pharmaceutically acceptable carrier or excipient. The composition is optionally formulated for direct intracranial administration, intracranial injection, intracerebroventricular injection, intracisternal injection, intrathecal injection, intravenous injection, intraperitoneal injection, delivery via nanoparticles, delivery via extracellular vesicles, and/or administration using focused ultrasound.

To gain a better understanding of the invention described herein, the following examples are set forth. It should be understood that these examples are for illustrative purposes only. Therefore, they should not limit the scope of this invention in any way.

### EXAMPLES

### Example 1

In this Example lentiviral delivery of Olig2, Sox10, or Nkx6.2 was used to show that single TFs can convert postnatal (P0-P5) GFAP+cortical astrocytes to different types of iOLCs. With live cell imaging, and single cell RNA sequencing (scRNA-seq) it was further demonstrated that each of these single TF-based DLR strategies converts different types of astrocytes, and follows different paths to iOLC generation. Altogether, these results demonstrate that donor astrocyte cell type and transcription factor choice converge to produce specific types of target oligodendrocyte lineage cells.

### Materials and Methods

### Experimental Design

The goal of this study was to demonstrate the role that the choice of transcription factor and donor cell heterogeneity have on the outcomes of DLR. In vitro DLR of astrocytes to oligodendrocytes was investigated using Olig2, Sox10, Nkx6.2 or a Cre control. The types and numbers of oligodendrocyte lineage cells produced were determined using immunocytochemistry over time and the process of DLR was analyzed using live cell imaging and single cell RNA sequencing. In the live cell experiments, cells that were not in the field of view for the entirety of the time course were not considered for analysis. In the single cell RNA sequencing experiment, microglia that were present in all treatment conditions were omitted from the final analysis. No outliers were identified.

### Animals

All experiments were performed in accordance with approved Animal Use Protocols from the Division of Comparative Medicine at the University of Toronto. Ai14 (B6;129S6-Gt (ROSA) 26Sor^{tm14(CAG-tdTomato)Hze}/J) mice were used for reactive astrocyte culture generation, reprogramming and live cell analysis. Ai14 mice were bred with S100β-EGFP (B6;D2-Tg (S100β-EGFP) 1Wjt/J) mice and these Ai14;S1006-EGFP mice were used for live cell analysis.

### Cell Culture

Cortical astrocytes were isolated from P0-P5 Ai14 mice as previously described (38). Briefly, mice were decapitated, followed by the removal of the skull and meninges. Cortices were dissected and mechanically dissociated in astrocyte media [DMEM (Gibco 10569-010), 10% fetal bovine serum (FBS) (Gibco 10082147) and 1% penicillin/streptomycin (Gibco 15140122)]. Cells were cultured in flasks pre-coated with 10 μg/ml poly-d-lysine (Sigma P6407), and incubated at 37° C., 5% CO₂. Media was changed the day following isolation and every other day thereafter. Once the cells reached 80% confluency, typically after 6 days, flasks were placed on an orbital shaker for 30 minutes at 180 rpm to remove contaminating microglia. Astrocyte media was replaced, and flasks were returned to the orbital shaker overnight at 180 rpm followed by vigorous shaking for one minute to remove contaminating OPCs and OLs. Media was removed and astrocytes were incubated in TrypLE Express Enzyme (Gibco 12604013) for 5 minutes at 37° C., 5% CO2 to lift off the astrocytes. To inactivate the enzyme, astrocyte media was added at a 3:1 ratio (media: TrypLE). Cell suspension was collected and centrifuged at 300×g for 5 minutes. Following removal of the supernatant, the pellet was resuspended in astrocyte media. Cells were then plated on poly-l-ornithine/laminin coated coverslips at either 50,000 or 70,000 cells/well in 24 well plates and incubated at 37° C., 5% CO2. For live cell analysis, cells were plated at 300,000 cells/well in 6 well plates and at 10,000 cells/well in 96 well plates, both with poly-I-ornithine/laminin coating.

For poly-I-ornithine/laminin coating, 0.1 mg/ml poly-I-ornithine (Sigma P4957) was added overnight at 37° C., washed 3 times with 1X phosphate buffered saline (PBS), and then incubated for two hours at 37° C. with 10 μg/ml laminin (Sigma L2020).

### Reprogramming

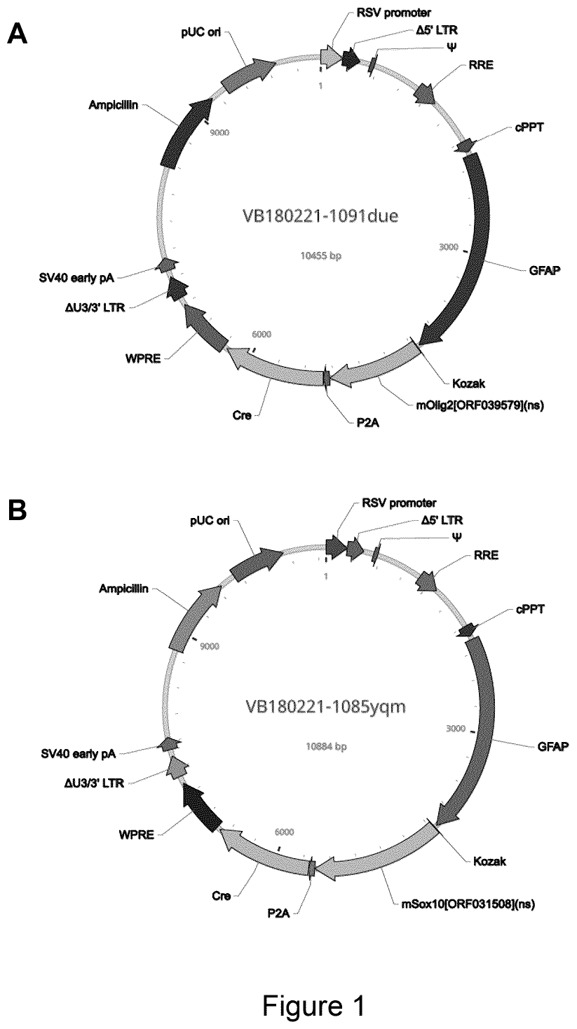

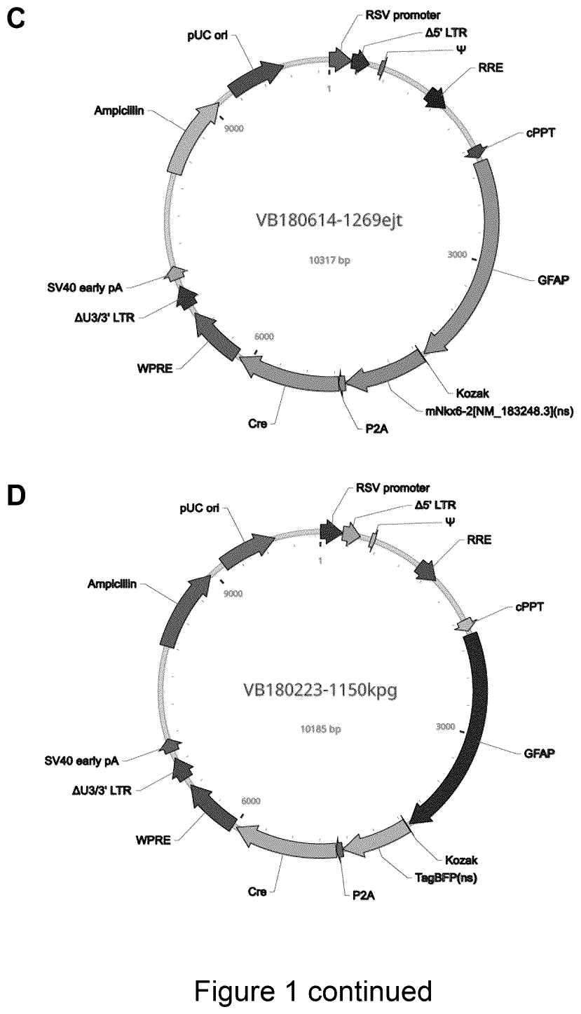

Lentiviral particles purchased from VectorBuilder were used to deliver the transcription factor of interest or control BFP linked to a Cre under the control of gfaABC (1) D, the 681 bp GFAP promoter, (LV-GFAP: : Olig2-P2A-Cre (SEQ ID NO:4); LV-GFAP: : Sox10-P2A-Cre (SEQ ID NO:5); and LV-GFAP: : Nkx6.2-P2A-Cre (SEQ ID NO:6), referred to as LV-GFAP: : Olig2, LV-GFAP:: Sox10, and LV-GFAP: : Nkx6.2, respectively; and LV-GFAP: : TagBFP-T2A-Cre (SEQ ID NO:7), referred to as LV-GFAP: : Cre) to astrocyte cultures. Schematic representations of these vectors are shown in FIG. 1A-D. A multiplicity of infection (MOI) of 100 was used for all experiments. Virus-containing astrocyte media was placed on the cells and left overnight. Viral media was replaced with fresh astrocyte media one day post transduction (DPT). Three DPT, cells were switched to OPC differentiation media (6) [DMEM/F12/Glutamax™ (Gibco 11330032), 1X N2 (Gibco 17502048), 1X B27 without vitamin A (Gibco 17504044), 200 ng/ml SHH (R&D Systems 464-SH), 20 ng/ml FGF (R&D Systems 3139-FB), 20 ng/ml PDGF (Sigma SRP3228)]. At 10 DPT, the cells were switched to OL differentiation media (6) [DMEM/F12/Glutamax™, 1X N2, 1X B27 without vitamin A, 40 ng/ml T3 (T2877 Sigma), 200 ng/ml SHH, 100 ng/ml Noggin (R&D Systems 1967-NG), 10 UM cAMP (Sigma A9501), 100 ng/ml IGF (R&D Systems 791-MG), 10 ng/ml NT3 (Sigma SRP6007)].

### Live Cell Analysis

For assessment of the reprogramming process and cell origin, transduced 96 well plates were imaged every hour for six days from 6 to 12DPT, using the Apotome live cell system (Zeiss). Four z-stack tiled images per well were captured every hour with brightfield and the 568 nm fluorescent wavelength. Images were stitched to create a continuous video for each well. At 12DPT, cells were fixed. Cells with oligodendrocyte lineage morphology at 12 DPT were analyzed for astrocyte or OLC morphology, and cell division from 6-12 DPT. OLC morphology was considered as either a round cell body with thin bipolar processes (OPC) or a round cell body with multiply, thin, branched processes (OL). Astrocyte morphology was considered as a star shaped cell body that was flat or branched.

### Immunocytochemistry

Cells were fixed in 4% paraformaldehyde (PFA) (Sigma P6148) for 20 minutes followed by three washes with 1×PBS. Cell membranes were permeabilized with 0.1% Triton-X-100 (Sigma X100) for 10 minutes at room temperature (RT), followed by three washes with 1×PBS, and then blocked with 5% milk for one hour at RT. Cells were incubated with primary antibodies in 1×PBS overnight at 4° C., washed three times with 1×PBS, and then incubated with secondary antibodies and DAPI in 1×PBS at RT for one hour. Following three final 1X PBS washes, coverslips were mounted on glass slides (Fisher Scientific 125523) with Mowiol mounting solution (Sigma 81381). For staining of membrane bound proteins (04, PDGFRa), no permeabilization step with Triton-X-100 was performed.

Primary antibodies: rabbit anti-GFAP (Dako Z0334), rabbit anti-OLIG2 (Millipore AB9610), mouse anti-SOX10 (Santa Cruz sc-365692), rabbit anti-PDGFRa (Abcam ab203491), mouse anti-04 (R&D systems MAB1326, 1:1000) and rat anti-MBP (Abcam 7349, 1:50)

Secondary antibodies: anti-mouse, anti-rabbit and anti-rat secondary Alexa Fluor 488 and 647 (Invitrogen, 1:1000).

### Microscopy and Image Analysis

Fluorescent images for quantification were taken on an LSM 880 Elyra

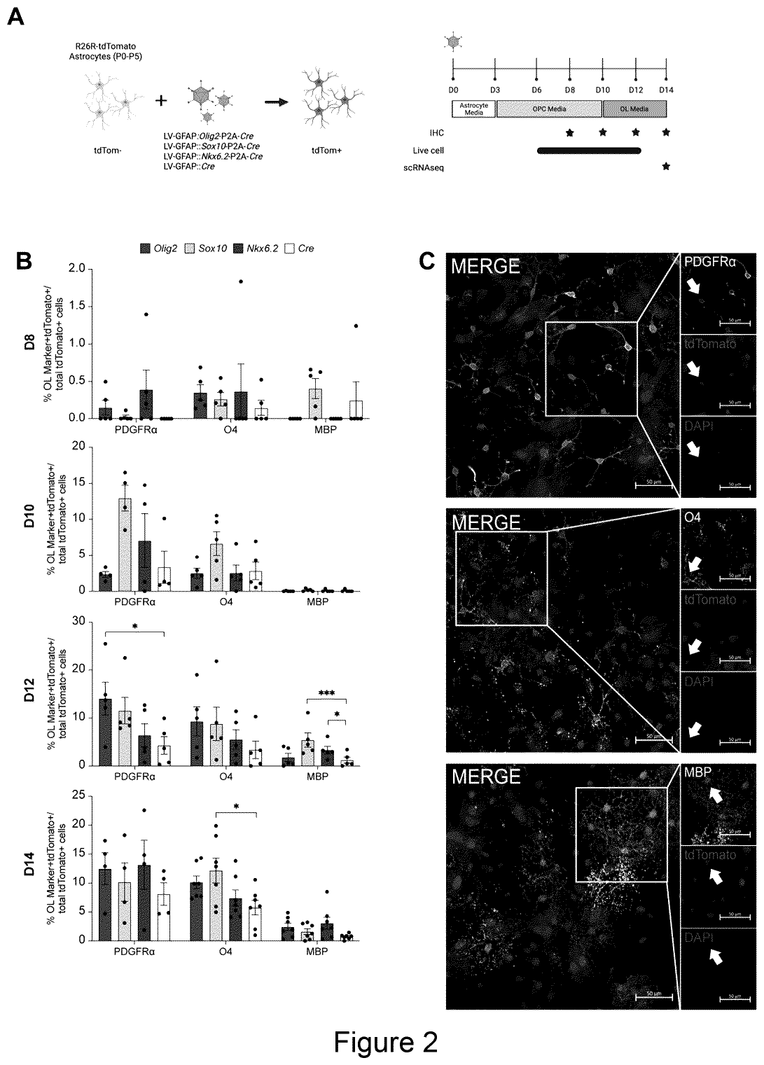

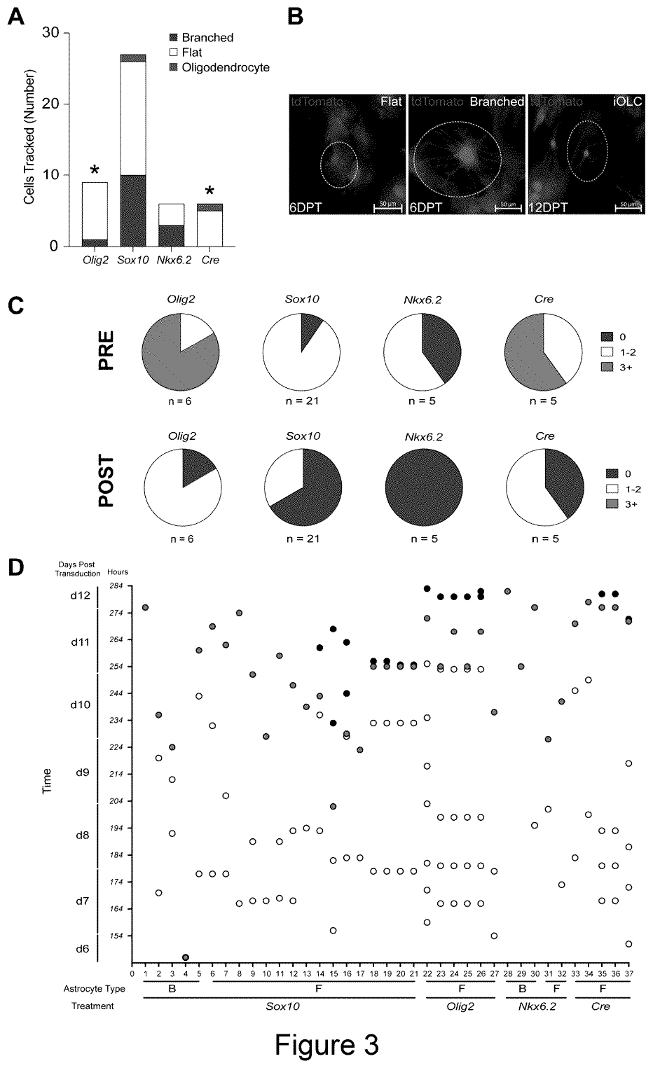

Superresolution and ZEISS LSM 900 (Zeiss) using a 20× objective and Zen software (Zeiss). Post-acquisition linear adjustments of brightness were made to micrographs using the Zen software in FIG. 2C and FIG. 3B). Threshold intensity was set according to the background signal detected in controls. For quantification, ten regions of interest were blindly selected at random for each well using DAPI fluorescence as reference. Images were analyzed using ImageJ software (National Institutes of Health). Transduction efficiency was calculated as a measure of total tdTomato⁺DAPI⁺ cells over total DAPI⁺ cells. Reprogramming efficiency was calculated as a measure of total OPC/OL markerttdTomato+DAPI⁺ cells over total tdTomato⁺DAPI⁺ cells.

### Single-Cell RNA-Seq

At 14DPT, LV-GFAP: Sox10, LV-GFAP: Olig2, LV-GFAP: : Nkx6.2, and control LV-GFAP:: Cre cultures were processed using the BD Rhapsody™ system (BD Biosciences) and then sequenced. For single-cell isolation, an average of 9814 viable cells were captured in wells at load. The BD Rhapsody™ scanner reported an average multiplet rate of 10.13% and an average number of wells with viable cells and a bead of 7081. Detailed metrics for each sample can be found in the Table 1 below.

TABLE 1

| | Control | Sox10 | Olig2 | Nkx6.2 | Average |
|---|---|---|---|---|---|
| Cell multiplet rate | 10.10% | 10.10% | 7% | 13.30% | 10.13% |
| Number of viable cells captured | 6726 | 12127 | 6733 | 13669 | 9813.75 |
| in wells at cell load | | | | | |
| Number of wells loaded with | 5967 | 10582 | 6134 | 11546 | 8557.25 |
| viable cells at cell load | | | | | |
| Number of wells with viable cells | 4943 | 9322 | 5562 | 8499 | 7081.5 |
| and a bead | | | | | |

Samples were down-sampled to approximately 2000 cells and carried through and converted to cDNA using the BD Rhapsody™ WTA Reagent Kit (Becton Dickinson Canada, Cat No. 633802). Each cell was sequenced at approximately 50,000 reads per cell, at 2×150 bp on a Novaseq™ (Donnelly Sequencing Centre, University of Toronto).

scRNA-Seq Analysis

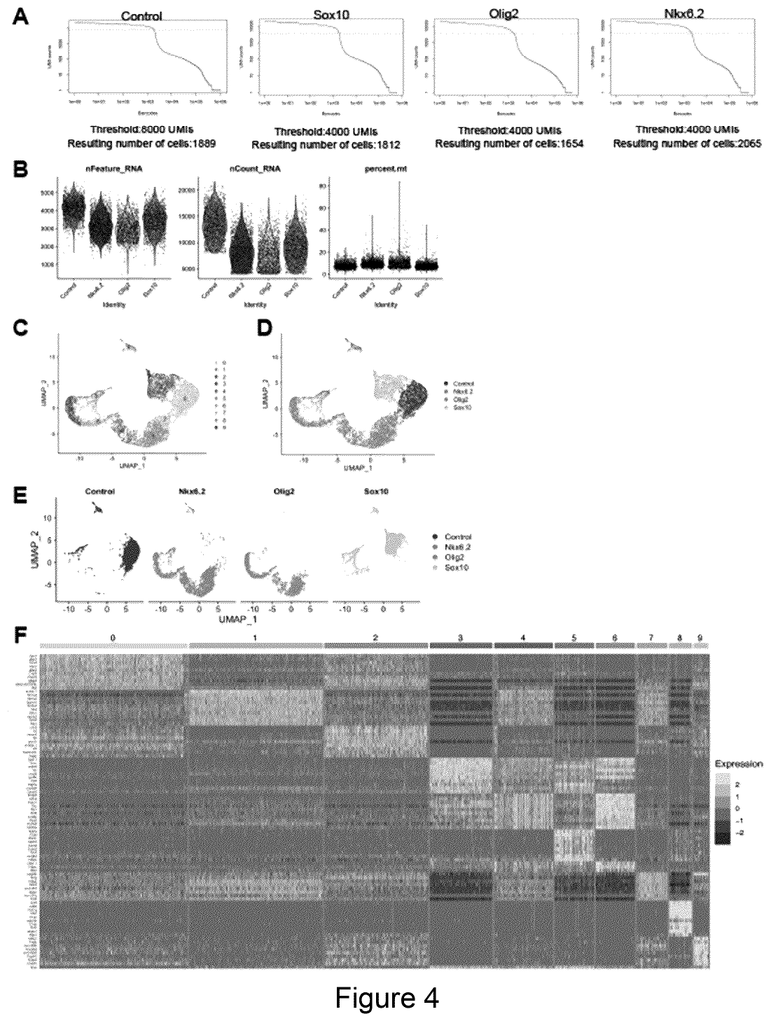

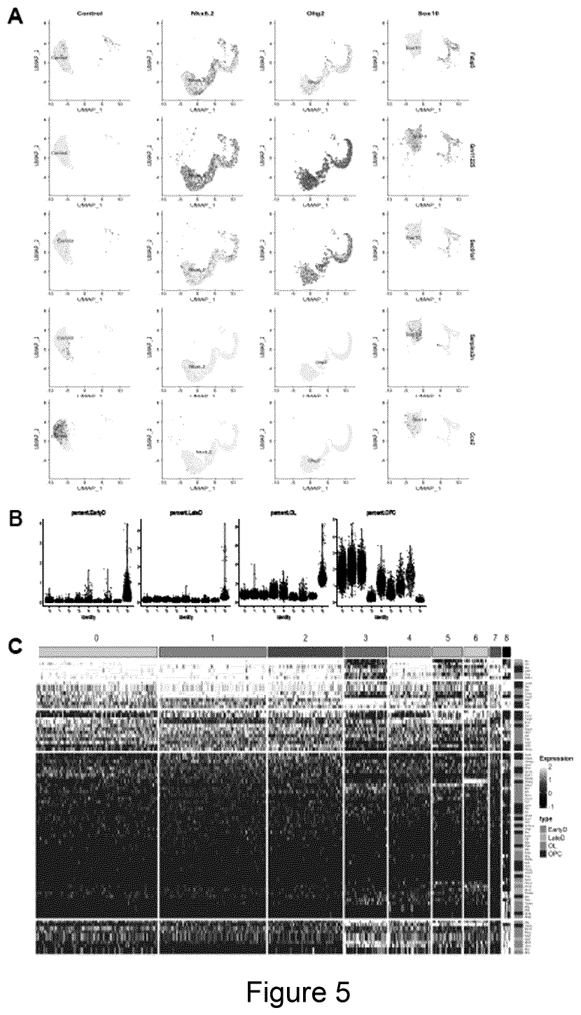

Fastq files were first demultiplexed with Kallisto (39) (v0.48.0) and Bustools (40) (V 0.41.0) using supplied whitelists with the -BDWTA option and aligning to GRCm38.96. Bustools (40) was then used to generate gene count tables. Cells were plotted based upon UMI counts per barcode, thresholds were selected based on inflection point of UMI count per barcode plots (FIG. 4A). These thresholds produced read and gene count distributions that were comparable between all treatment groups (FIG. 4B). Gene count tables were made into S4 objects, scaled, normalized and dimensions reduced (PCA then UMAP) using the Seurat package (41) (v4.1). Upon clustering there was a distinct cluster identifying as microglia, (FIG. 4C-F), cluster 8, which was removed for further analysis. Gene markers for oligodendrocyte lineage cells were adopted from studies observing in vivo mouse oligodendrocyte lineage cells across several areas in young and mature CNS tissues (33). These markers were converted into percent expression of each UMAP cluster using the PercentageFeatureSet function from Seurat (41), with further gene resolution displayed by heatmaps created using ComplexHeatmap (42) (v12.13.1). An additional set of gene markers, demonstrating similar, but less resolved, conclusions was also used from an in vitro rat study looking at OLCs from the cortex (34) (FIG. 5A-B). Astrocyte markers (35) were scored, using AddModuleScore function from Seurat (41), and overlayed onto UMAP plots. Trajectories were inferred using Slingshot (43) (v2.3.1) and differential genes along trajectories using random forest models from tidymodels (44) (v0.2.0) and Parsnip (45) (v0.2.1). Plots were produced using ggplot2 (46) (v3.3.5) and figure generating scripts were run in R studio (47) (v4.2.0), with demultiplexing using Kallisto (39) and Bustools (40) run on a Compute Canada HPC cluster. All scripts used for processing of scRNA-seq data and for figure generation can be found at github.com/eyscott.

### Statistical Analysis

Percentage values were transformed using the arcsine square root transformation and assessed for normal distribution and variance using the Shapiro-Wilks test and Levene's test, respectively. When distribution and variance were equal, a matched pairs one-way ANOVA (D8, D10, D12) or one-way ANOVA (D14) was performed to compare reprogramming efficiency of TF groups to a control group (LV-GFAP: : Cre). When transformed values did not follow a Gaussian distribution, a Kruskal-Wallis nonparametric test was performed to compare reprogramming efficiency to a control group (LV-GFAP: : Cre). In both cases, Dunnett's post-hoc testing was performed to correct for multiple comparisons. Comparison of percentages between groups was done using two sample z-tests for individual proportions. Differences were considered significant at p<0.05. Values are presented as mean±SEM. The statistical software used was GraphPad Prism version 9.0.1 (48).

### Results

Different Types of iOLCs are Generated Via DLR with Olig2, Sox10 or Nkx6.2

To examine the reprogramming potential of Olig2, Sox10, and Nkx6.2, postnatal, cortical astrocyte cultures from Ai14 mice were transduced with either LV-GFAP: : Olig2, LV-GFAP:: Sox10, LV-GFAP: : Nkx6.2 or a control LV-GFAP: : Cre. To assess the purity of the cultures, the percentage of contaminating tdTomato⁺OLCs in control Cre-transduced cultures at 3 days post transduction (DPT) was quantified. Negligible numbers of tdTomato⁺SOX10+, tdTomato⁺O4+ and tdTomato⁺MBP+OLCs were found. However, 66.63% of tdTomato⁺ cells expressed OLIG2, in line with previous studies showing that astrocyte subsets express OLIG2 (9, 22).

To determine the ability of Olig2, Sox10, and Nkx6.2 to reprogram astrocytes, the numbers of tdTomato⁺ transduced cells at 8, 10, 12 and 14 DPT that co-expressed markers of OPCs, COPs, and myelin forming OLs (mfOLS) were quantified (FIG. 2A). By 8 DPT tdTomato⁺ cells that showed classic OL lineage morphology (a round cell body with thin processes) and markers of iOLCs were present in all cultures. However, at both 8 and 10 DPT, no differences were seen in the percentage of tdTomato⁺PDGFRa⁺ iOPCs, tdTomato⁺O4⁺ iCOPs, or tdTomato⁺MBP+ imfOLs in TF-transduced cultures compared to controls (FIG. 2B). This suggested that there is baseline astrocyte to iOLC conversion following astrocyte exposure to OLC media.

By 12 DPT an increase in iOLCs in TF-transduced cultures was observed relative to controls. Olig2-cultures showed an increase in the percentage of tdTomato⁺PDGFRa⁺ iOPCs (FIG. 2B-C), whereas Sox10- and Nkx6.2-cultures showed an increase in tdTomato⁺MBP⁺ imfOLs (FIG. 2B-C). No differences were seen in the percentage of tdTomato⁺O4⁺ iCOPs in any condition at 12DPT (FIG. 2B-C).

At 14DPT, there were no differences observed in the percentage of tdTomato⁺PGDFRa⁺ iOPCs or tdTomato⁺MBP⁺ imfOLs, however, Sox10-cultures showed higher percentages of tdTomato⁺O4⁺ iCOPs (FIG. 2B).

An important functional aspect of OLs is their ability to myelinate. To determine whether the lack of tdTomato⁺MBP⁺ imfOLs in Olig2-cultures was due to insufficient time in culture, cells were cultured in OL differentiation media for an additional 8 days and then the cells were analyzed at 22 DPT. No differences were seen in tdTomato⁺MBP⁺ imfOLs in any of the conditions.

Altogether, these findings show that different TFs generate iOLCs at different stages in the OL lineage. Olig2 converts astrocytes to iOPCs, Sox10 can promote two distinct phenotypes, myelinating (imfOLs) and pre-myelinating (iCOPS) but along different time courses, while Nkx6.2 generates imfOLs.

iOLCs Arise from Bonafide Astrocyte Conversion

To confirm whether the iOLCs are a result of astrocyte conversion, live cell imaging of Olig2-, Sox10-, Nkx6.2-, and control cultures was performed. Cells were imaged every hour from 6DPT-12DPT in order to capture cells both prior to, and during the process of reprogramming (FIG. 2A). To track cell conversion, cells were identified at 12DPT as iOLCs based on morphology and their conversion was observed retrospectively to 6DPT. iOPCs had a round nucleus with bipolar thin processes, while iOLs had a round nucleus and multiple branched thin processes. Of the 42 cells tracked in Olig2, Sox10, and Nkx6.2-cultures, all but one arose from astrocytes, indicating that the TF-derived iOLCs observed at 12 DPT result from astrocyte conversion (FIG. 3A). Of the six Cre-transduced control cells tracked, five also arose from astrocytes and one from a contaminating OL lineage cell (FIG. 3A), which confirmed the baseline level of astrocyte conversion previously observed (FIG. 2B). Of note, the number of cells tracked per treatment was not indicative of the reprogramming efficiency of each factor, but rather due to frame rate limitations that preclude the tracking of some cells for the entire live cell time course.

The type of astrocyte converted is transcription factor-dependent

In the above live cell experiment, also observed were two predominate astrocyte morphologies at 6DPT from which iOLCs originated: i) a flat, multi-branched cell (referred to as branched), or ii) a flat cell with little to no branching (referred to as flat) (FIG. 3A-B). Conversion of branched astrocytes to iOLCs was only observed in TF-transduced cultures, while flat astrocytes were seen to convert in both TF- and Cre-transduced cultures (FIG. 3A). Although each TF could convert astrocytes with both morphologies, Olig2 preferentially converted flat astrocytes (branched: 1/9, flat: 8/9, z=−3.30, p=0.0096) (FIG. 3A). Without wishing to be bound by theory, this data suggested that branched astrocytes are only converted via TF-mediated reprogramming, and that different TFs may preferentially convert branched-or flat-types of astrocytes.

### Cell Division During DLR is TF-Specific

To understand whether DLR of astrocytes to iOLCs requires proliferation, the numbers and types of cell divisions in Olig2-, Sox10-, Nkx6.2-, and control cultures were analyzed from 6DPT-12DPT. In both Sox10- and Nkx6.2-transduced cultures, direct reprogramming was observed in non-mitotic cells (0 cell divisions) (FIG. 3C-D, cells 1, 4, 28 and 29), while in Olig2- and control transduced cultures, no conversion was seen in non-mitotic cells (FIG. 3C-D). Next the number of divisions in astrocytes prior to the acquisition of iOLC fate were compared, and it was found that 15/21 Sox10-transduced cultures underwent 2 cell divisions, 4/21 underwent a single cell division, and 2/21 cells reprogrammed directly (FIG. 3C-D). Nkx6.2-transduced cells showed little division prior to conversion, whereby 3/5 cells underwent 1 division and 2 converted directly (FIG. 3C-D). In contrast, Olig2- and Cre control-transduced cells underwent multiple divisions prior to conversion (Olig2: 1/6 cells, 5+ divisions, 4/6, 4 divisions, 1/6 1 division; Cre: 1/5, 4 divisions, 2/5, 3 divisions, 2/5, 2 divisions; FIG. 2C-D, Supplementary Video S1). When comparing the number of divisions after iOLC fate acquisition, 5/6 Olig2-transduced cells divided, compared to 7/21 Sox-10-transduced cells, 0/5 Nkx6.2-transduced cells and 3/5 Cre transduced cells (FIG. 2C-D). This supported the previous finding that Olig2 drives a mitotic iOPC identity while Nkx6.2 results in a non-proliferative imfOL identity (FIG. 2B). Overall, these findings demonstrate that the process of conversion and proliferative capacity of the transduced cells is also TF-dependent.

Olig2-, Sox10- and Nkx6.2-Direct Different Pathways to iOLC Conversion

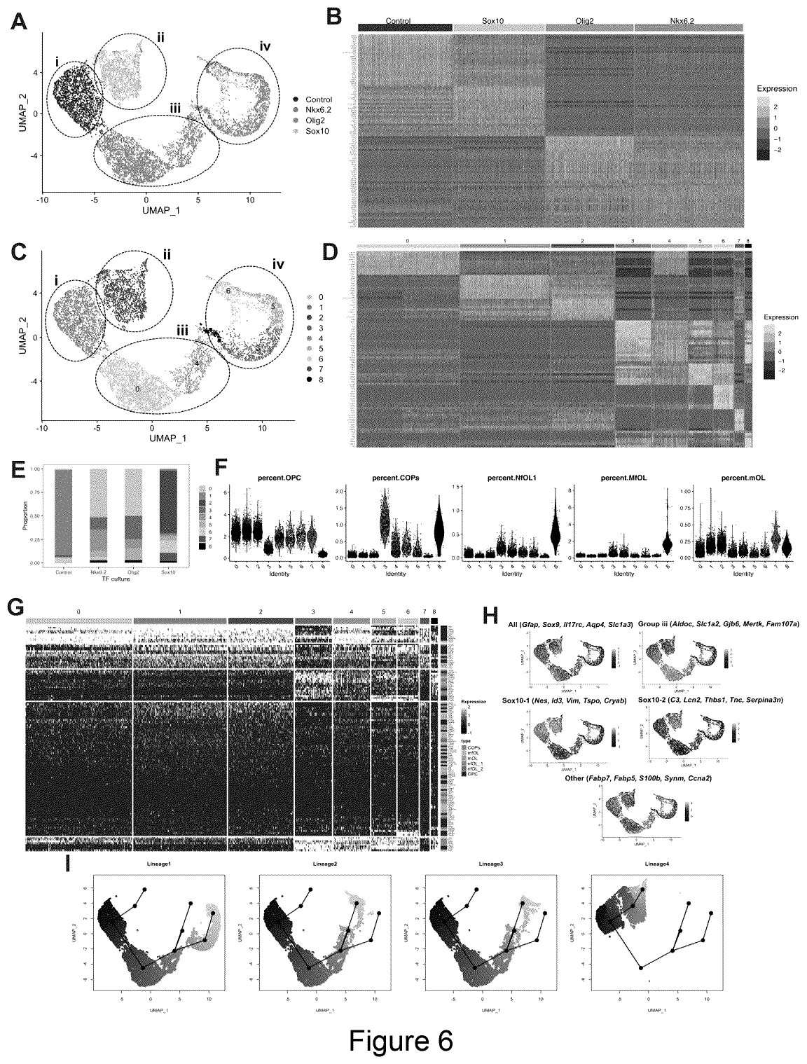

To further resolve the TF-based differences in DLR that were observed in the time course and live cell experiments, scRNA-seq was performed on Olig2-, Sox10-, Nkx6.2- and control cultures at 14DPT (FIG. 2A). Combined transcriptomic analysis showed four distinct groups comprised of: i) control cells, ii) Sox10-transduced cells, iii) subsets of Nkx6.2- and Olig2-transduced cells, and iv) cells from all four cultures, to varying degrees (FIG. 6A). Control cells (group i; astrocytes cultured in OL media) showed upregulation of genes that are important for shaping the OL niche. For example, matrix metalloproteases (Mt2) and their inhibitors (Timp3), which are secreted by astrocytes and can regulate OL differentiation and myelination (23), as well as Ccn2, shown to be important for OL maturation (24), 2017) (FIG. 3B). In Sox10-transduced cells (group ii), the enriched genes were associated with astrocyte reactivity [Nestin (25), Serpina3n and C3 (26)](FIG. 6B). In Nkx6.2- and Olig2-transduced cells (group iii), pan-astrocytic genes were upregulated (Slc1a2/Glast (27, 28) and Aldoc (29), and there was enrichment of reactive astrocyte genes that were common to Nkx6.2- and Olig2-cells (Vcan (30)), or expressed only in Nkx6.2-(Fabp5 (31)) or Olig2-cells (Sec61a1 (32)) (FIG. 6B, FIG. 5A).

To further understand the heterogeneity of the samples used, these broadly defined groups were clustered. The control cells (group i) remained as a single cluster (cluster 1); the Sox10-cells (group ii) separated into clusters 2 and 7, the Nkx6.2- and Olig2-cells (group iii) was divided into clusters 0 and 4, and the group with cells from all three TFs (group iv) was split into clusters 3, 5, 6 and 8) (FIG. 6C). Next, the gene expression in group iv was examined, as it included cells from all four cultures. Clusters 3, 5, 6, and 8 in group iv were characterized by canonical markers of the OL lineage (Plp1, Vcan, Gpr17, and Lhfpl3) (FIG. 6D). To understand whether different iOLCs originated as a result of specific TF expression, the relative proportion of the clusters within Sox10-, Olig2-, Nkx6.2-, and control cultures was examined. In each condition, a dominant cluster was found that encompassed over 50% of the transcription: cluster 1 was predominant in control cells (group i), cluster 2 in Sox10-cultures, (group ii) and cluster 0 in Nkx6.2- and Olig2-cultures (group iii) (FIG. 3E).

To understand how similar the iOLCs for this Example were to OLCs found in vivo, the dataset obtained from this study was compared to established OL lineage transcriptomic datasets (33, 34) (FIG. 6F-G, FIG. 5B-C). OL specific annotations of mouse fetal and adult OLCs derived from Marques et al, 2016, were used to bin the data. OPC-like signatures were found in all clusters, except cluster 3 and 8 (FIG. 6F). COP signatures were found in cluster 3 and to a lesser degree, cluster 8 (FIG. 6F). nfOL and mfOL signatures were seen in cluster 8 (FIG. 6F). Cluster 7, which was exclusively comprised of Sox10-induced cells (group ii), showed mature OL (mOL) signatures (FIG. 6C,F). This demonstrated that ectopic expression of Sox10 generated at least two mature forms of OLCs (mOL and mfOL) via unique paths.

When examining genes driving the OLC cell “types” determined by Marques et al, 2016, it was observed that a subset of OPC markers (Mt3, Ddah1, Ccnd2, Ccng1 and Glul) was predominantly expressed in clusters 0, 1, 2 and 4, while genes that belong to several OLC types (Plp1, Fyn, Vcan, Gpr17, Cd9 and Lhfpl3) were enriched in the clusters 3, 5, 6 and 8 (FIG. 3G).

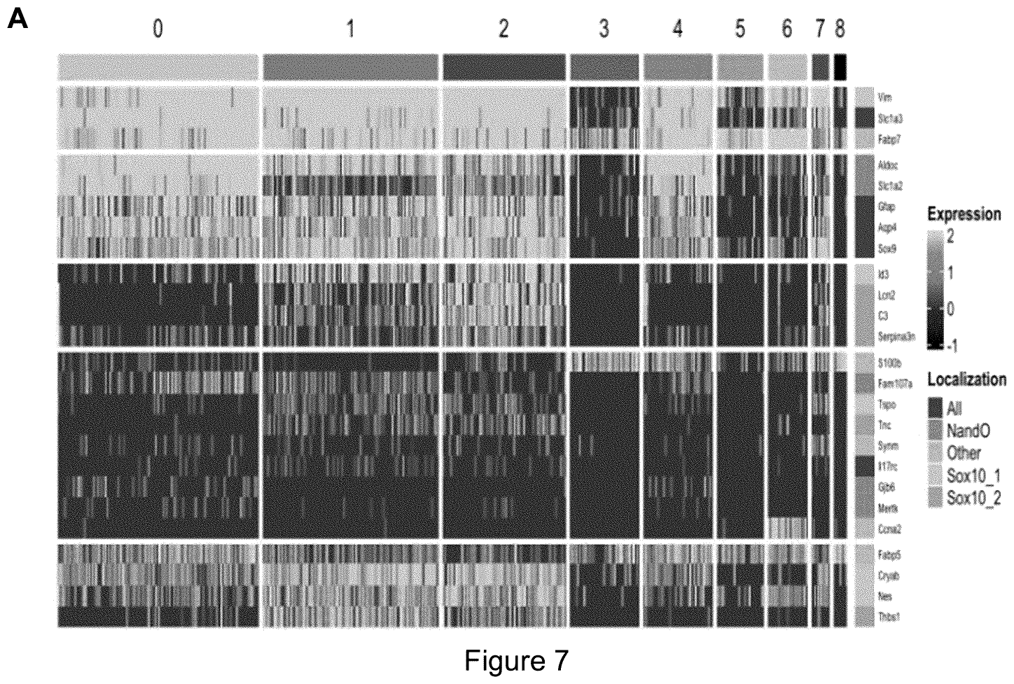

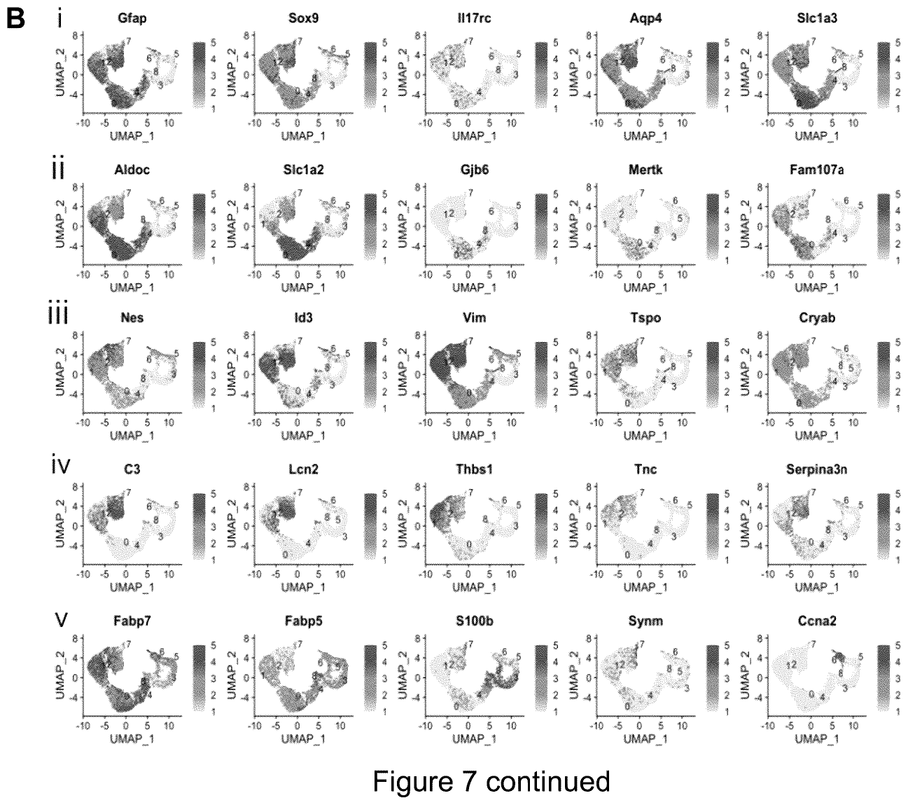

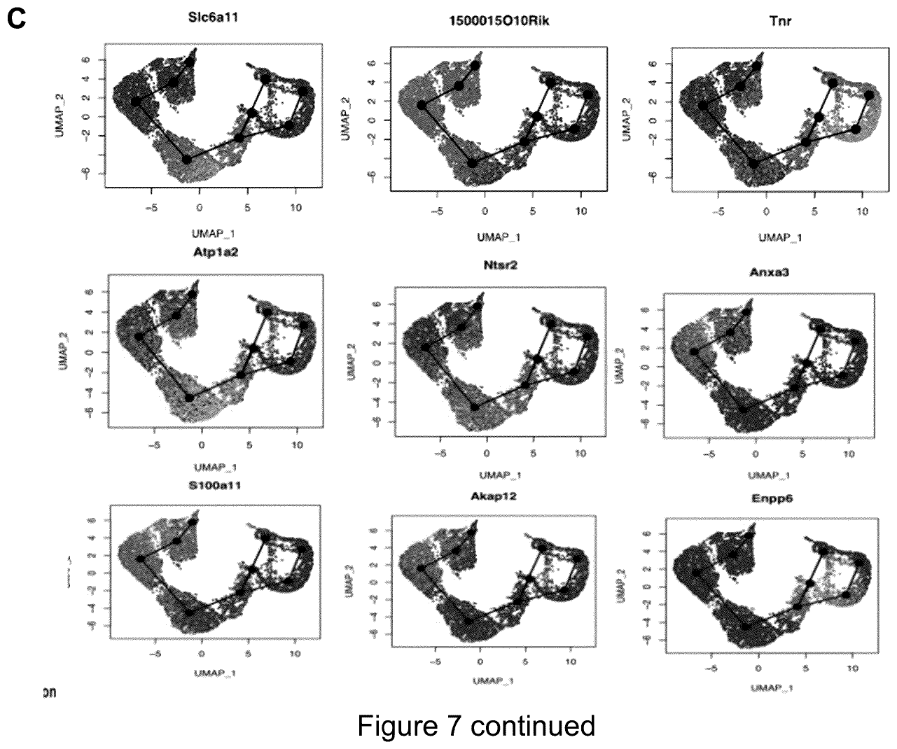

Given that different gene expression signatures related to astrocyte reactivity were observed in each culture, changes in astrocytes during DLR were further examined (FIG. 6H, FIG. 7A,Bi-v). When individual genes associated with astrocytes/astrocyte reactivity were overlaid onto the UMAP it was found that all cells in groups (i), (ii), and (iii) expressed pan astrocyte genes (Gfap, Sox9, Slc1a3), however, gene expression patterns were identified that were specific to each TF (FIG. 6H, FIG. 7A, Bi-v). Nkx6.2- and Olig2-cultures (group iii) were preferentially enriched with astrocyte genes Aldoc, Slc1a2, Gjb6, Mertk and Fam107a (FIG. 6H, FIG. 7A, Bii) that are associated with a mature astrocyte phenotype, and have roles in general brain homeostasis and flux, trophic and metabolic support for neurons, and modulating neuronal activity (35). Of interest, Sox10-cultures (group ii) showed two unique astrocytic signatures that occupied different positions within cluster 2. The first (Sox10-1) was driven by Nes, Id3, Vim, Tspo and Cryab, while the second (Sox10-2) was restricted to a subset of cells and driven by C3, Lcn2, Thbs1, Tnc and Serpina3n (FIG. 6H, FIG. 7A,Biii-iv). There was also a separate astrocyte gene signature (Fabp7, Fabp5, S100b, Synm and Ccna2) present in all groups (i-iv) that was absent from clusters 7, 8 and 3 (FIG. 6H, FIG. 7A,Bv). Altogether, these data illustrate that specific TFs push astrocytes into different states.

Next, lineage analysis was performed on the present scRNA-seq dataset to differentiate between intermediate and terminal stages of DLR. As expected, the trajectories showed a progression of reprogramming. Clusters 0, 2, 4 represented intermediate stages of DLR, while clusters 5,6,7,8 represented end stages of DLR ((FIG. 6I). This supports the previous findings that group iv (clusters 3, 5, 6 and 8) represent more complete reprogramming to iOLCs, while groups ii and iii (clusters 2, 7 and 0, 4) represent astrocytes that were not fully reprogrammed. In addition, through analysis of the genes driving these trajectories, the inventors were able to identify additional gene candidates for iOLC reprogramming (FIG. 7C). Taken together, the lineage analysis showed the presence of transitory versus more differentiated iOLC cell states within the present scRNA-seq data and showed that TF-specific paths are taken during the reprogramming process.

### DISCUSSION

DLR, via ectopic expression of TFs, can generate new cells for CNS repair. Here, the inventors, have demonstrated that the single TFs, Sox10, Olig2, and Nkx6.2, can be used to reprogram astrocytes to iOLCs. While previous studies demonstrated the generation of iOLCs from different types of somatic cells using components or combinations of Sox10, Olig2 and Nkx6.2 (6, 7, 36), this is the first demonstration of the individual reprogramming ability of each these TFs and the associated processes. Importantly, it has now been shown that Sox10, Olig2 and Nkx6.2, direct the generation of different types of iOLCs, target different astrocytes, and shift astrocytes into specific states and along specific pathways that are unique to each TF.

Among the three TFs, the inventors have demonstrated that Sox10 produced two types of OLCs at different timepoints, MBP⁺ imfOLs at 12DPT and O4⁺ iCOPs at 14DPT. This suggested at least two different paths to reprogramming, which was supported by scRNAseq data that showed Sox10-shifts astrocytes into two different states and along two different lineage trajectories during conversion to iOLCs. In a previous study, Sox10 was also used as a single factor for astrocyte to OLC conversion, but Khanghahi et al. found that Sox10 expression in astrocytes in vitro led to an increase in OPCs at 21 DPT (20), a different cell type and time course than observed in this study. Although the primary cells were comparable [P3-P5 astrocytes cultured in OPC media (20) versus this study using P0-P5 cortical astrocytes in OPC then OL media], the differences in the present studies may be due to the use of different delivery strategies and metrics of reprogramming. In the present study, the inventors have used a lentiviral delivery strategy in which Sox10 was delivered under the control of a GFAP promoter and linked to a Cre recombinase. This resulted in the permanent labelling of astrocytes (derived from Ai14 mice) with tdTomato and enabled tracking of the fate of transduced cells and quantify iOLCs (based on tdTomato+iOLC marker+expression). In contrast, in the Khanghahi et al study, the authors used a SSFV promoter to deliver Sox10 linked to a GFP reporter and reported the number of GFP+iOLCs in the Sox10-transduced cultures. Without an astrocyte specific promoter, a contaminating GFAP-, more distantly related cell, may have been transduced that would need more time to convert. Without wishing to be bound by theory this could explain the differences in timing and iOLC cell type generated in the present studies.

The present inventors have shown that different TFs result in different iOLC lineage cells. Olig2-results in PDGFRa⁺ iOPCs at 12DPT, which is consistent with the findings from live cell experiments that suggested Olig2 induces a proliferative iOLC type (many cell divisions post conversion). In contrast, it was found that Nkx6.2 results in a more mature MBP⁺ imfOL identity at 12DPT, which corresponds to a non-proliferative phenotype (no cell divisions post-conversion). These differences were not present at 14DPT, the time point of the scRNA-seq analysis, which could explain the closely related transcriptomes observed between Olig2- and Nkx6.2-cultures.

Although it was found that there was baseline conversion in the present cultures, likely due to media favoring OLCs, the present data demonstrates that ectopic expression of TFs results in distinct types of DLR that are more specific and robust. First, analysis of iOLCs at 12 and 14 DPT showed a clear increase in the percentage of iOLCs. Second, proliferation analysis showed that only Nkx6.2 and Sox10 were able to convert non-mitotic cells (no cell divisions pre-iOLC conversion). Third, scRNA-seq analysis demonstrated that control cells are a relatively homogeneous population that is transcriptomically separate from TF-induced cultures, and largely characterized by astrocyte gene expression.

An important aspect of DLR is that the generation of a new cell is accompanied by the removal of a donor cell. Astrocytes are an attractive donor cell type for DLR in the CNS, as astrocytes respond to all forms of neurological injury and disease and their proximity to the site of injury or damage makes them a promising target for conversion. In addition, the removal of specific types of astrocytes that drive disease pathology can improve disease outcomes. However, the removal of astrocytes in DLR has been criticized (37) as it is very well established that astrocytes can aid in the recovery from injury and offer benefit in disease. Therefore, it is of importance that the present data establishes that DLR can target different types of astrocytes, as this allows for more specific, tailored therapeutic strategies that do not remove all astrocytes during DLR. The present live imaging experiments showed that TF-based DLR can convert both flat and branched astrocytes, and that Olig2 preferentially targets astrocytes with flat morphology.

The inventors' finding that different TFs push astrocytes to different states, that are classified herein as reprogramming intermediates, is particularly interesting. Many of the genes that were enriched in some of these astrocyte intermediates have previously been associated with reactivity. In particular, genes enriched in Sox10-cultures (group ii) are associated with immune challenge (26) and genes in Nkx6.2- and Olig2-(group iii) cultures (Aldoc, Slc1a2, Gjb6, Mertk and Fam107a) have primarily been associated with a mature, physiological astrocyte type (35).

In conclusion, this Example showed that TF choice influences DLR to iOLCs. These results provide the ability to tailor DLR therapies to generate specific types of OLCs and target specific types of astrocytes according to disease need. These findings provide a means to provide therapeutic benefit of astrocyte to OL DLR using Olig2, Sox10, or Nkx6.2 in different types of CNS disease and injury.

### Example 2: Direct Lineage Conversion of Postnatal Mouse Cortical Astrocytes to Oligodendrocyte Lineage Cells

Oligodendrocyte lineage cells (OLCs) are lost in many CNS diseases. Direct lineage reprogramming (DLR) of astrocytes can generate new cells for CNS repair. This example demonstrates the conversion of mouse postnatal astrocytes to OLCs by ectopic expression of Sox10, Olig2 and Nkx6.2. Using stringent analyses including, Aldh1l1-astrocyte fate mapping and live cell imaging these results confirm that Sox10 and Olig2 directly convert Aldh1l1POS astrocytes to MBP+ and PDGFRax+ induced OLCs (iOLCs), respectively. The molecular signatures of iOLCs were uncovered with single cell RNA sequencing (scRNA-seq). Transcriptomic analysis of Sox10- and control cultures over time revealed a clear trajectory from astrocytes to iOLCs. Finally, perturbation models CellOracle™ and Fatecode™ support the idea that Sox10 drives cells towards a terminal iOLC fate. Altogether, this multidimensional analysis shows bonafide conversion of astrocytes to iOLCs using Sox10 or Olig2 and provides a foundation for use of astrocyte DLR strategies to promote OLC repair.

Oligodendrocytes (OLs) are best known as the myelinating cells of the central nervous system (CNS). OLs ensheath neuronal axons to enable fast propagation of action potentials. Consequently, the loss or dysfunction of OLs results in impaired neurological function that is characteristic of many types of CNS disease and injury. Multiple sclerosis (MS), Alzheimer's Disease, spinal cord injury, white matter stroke and cerebral palsy are all characterized by oligodendrocyte failure. Thus, therapeutic strategies aimed at replacing OLs are of significant clinical interest. (49)

Direct lineage reprogramming (DLR) aims to generate new target cells lost to disease via the forced conversion of donor cells. Typically, DLR is performed by the overexpression of transcription factors. Early pioneering work from the Tesar Lab demonstrated fibroblast to induced oligodendrocyte progenitor cell (iOPC) conversion by ectopic expression of Olig2, Sox10, and Nkx6.2, determinants of OL cell fate in the embryonic brain. (6) The combination of Sox10 and Olig2 was also later used to generate iOPCs from pericytes. (7) Therapeutically, however, these newly generated iOPCs still require transplantation into the brain. Therefore, methods to reprogram endogenous CNS cells would be advantageous.

In parallel, astrocytes, CNS-resident cells, have emerged as donor cells in DLR strategies aimed at generating new neurons. (1,3,50,51) Astrocytes are an attractive donor cell type for OL conversion given their shared neural origin. (16,17) Astrocytes may already have relevant epigenetic marks and active TFs that could make DLR faster or more efficient. (18,19,52,53) In addition, closely related cells may require fewer TFs for conversion. Indeed, conversion of astrocytes to an induced oligodendrocyte-like cell was suggested using Sox10 alone. (20) Therefore, single ‘Tesar’ factors, Olig2, Sox10, or Nkx6.2, were identified by the present inventors to be studied to force astrocyte conversion to new, induced oligodendrocyte lineage cells (iOLCs).

The OL lineage is comprised of oligodendrocyte progenitor cells (OPCs) that give rise to committed oligodendrocyte progenitors (COPs). These COPs differentiate into mature OLs (mOLs), comprising at least 6 different states. (33) Of interest, during OL development, each of these ‘Tesar’ factors shows different temporal expression and plays a different role in OL fate specification. Olig2, long considered an OL lineage fate determinant, (8) is also expressed in astrocytes, (54,55) which suggests a broad role in early glial commitment. (9,10) Sox10 is important throughout OL development; Sox10 promotes early OL lineage specification by re-inducing Olig2 (11) and by inhibiting Sufu, (12) but is also required for OL survival following myelination. (13,56) In contrast, Nkx6.2 is expressed late in OL development, with myelin genes Mbp and Mog, and plays a role in regulating myelination. (14,15) Given the unique roles of each of these TFs in development, the present inventors have studied the use of single Tesar factors to create distinct types of iOLCs that ranged from OPCs to mature, myelinating OLs.

Recent studies have highlighted the need for rigorous reporting of DLR outcomes, following controversy of astrocyte to neuron DLR in vivo. A landmark study using astrocyte fate mapping strategies suggested that conversion was misrepresented as a result of AAV and promoter confounds. (57) Therefore, it is important that new DLR paradigms utilize fate mapping, and stringent, multi-faceted analysis to determine the origin of the newly generated cells.

In the present example, lentiviral delivery of Olig2, Sox10, or Nkx6.2 was used to investigate single TF conversion of postnatal (P0-P5) GFAP+cortical astrocytes to different types of iOLCs. Lineage tracing experiments using Aldh1l1-CreERT2;Ai14 mice demonstrated that Sox10 and Olig2 convert Aldh1l1+astrocytes to iOLCs. Moreover, live cell imaging, single cell RNA sequencing (scRNA seq) and deep learning methods further support the findings that iOLCs can be generated from astrocytes following TF delivery. Altogether, our findings show bonafide astrocyte to iOLC DLR and support the use of DLR for treating diseases involving OLC dysfunction and loss.

### Methods

### Animals

All experiments were performed in accordance with approved Animal Use Protocols from the Division of Comparative Medicine at the University of Toronto. P0-P5 Ai14 (B6;129S6-Gt (ROSA) 26Sor^{tm14(CAG-tdTomato)Hze}/J, RRID: IMSR_JAX: 007908) and Ai14;Ald1l1-CreERT2 mice (Ai14 crossed to B6N.FVB-Tg (Aldh1l1-cre/ERT2) 1Khakh/J [RRID: IMSR_JAX: 031008]) were used to generate postnatal astrocyte cultures.

### Cell Culture

Cortical astrocytes were isolated from male and female P0-P5 mice as previously described³⁸. Briefly, mice were decapitated, followed by the removal of the skull and meninges. Cortices were dissected, pooled, and mechanically dissociated in astrocyte media [DMEM (Gibco Catalogue No. 10569-010), 10% fetal bovine serum (FBS) (Gibco Catalogue No. 10082147) and 1% penicillin/streptomycin (Gibco Catalogue No. 15140122)]. Cells were cultured in flasks pre-coated with 10 μg/ml poly-d-lysine (Sigma Catalogue No. P6407), and incubated at 37° C., 5% CO₂. Media was changed the day following isolation and every other day thereafter. Once the cells reached 80% confluency, typically after 6 days, flasks were placed on an orbital shaker for 30 minutes at 180 rpm to remove contaminating microglia. Astrocyte media was replaced. For Ai14;Aldh1l1-CreERT2 cultures, 1 uM 4-OHT was added to the astrocyte media at this step. Flasks were returned to the orbital shaker overnight at 180 rpm, followed by vigorous shaking for one minute to remove contaminating OLCs. Media was removed and astrocytes were incubated in TrypLE Express Enzyme (Gibco Catalogue No. 12604013) for 5 minutes at 37° C., 5% CO₂ to lift off the astrocytes. To inactivate the enzyme, astrocyte media was added at a 3:1 ratio (media: TrypLE). Cell suspension was collected and centrifuged at 300×g for 5 minutes. Following removal of the supernatant, the pellet was resuspended in astrocyte media. Cells were then plated on poly-l-ornithine/laminin coated coverslips at either 50,000 or 70,000 cells/well in 24 well plates and incubated at 37° C., 5% CO₂. For live cell analysis, cells were plated at 10,000 cells/well in 96 well plates with poly-I-ornithine/laminin coating. For poly-I-ornithine/laminin coating, 0.1 mg/ml poly-I-ornithine (Sigma Catalogue No. P4957) was added to dishes overnight at 37° C., washed 3 times with 1X phosphate buffered saline (PBS), and then dishes were incubated for two hours at 37° C. with 10 g/ml laminin (Sigma Catalogue No. L2020).

### Reprogramming

Lentiviral particles were purchased from VectorBuilder. For Ai14 reprogramming, LV-hGFAP: : Sox10-P2A-Cre, LV-hGFAP: : Olig2-P2A-Cre, LV-hGFAP: : Nkx6.2-P2A-Cre, and control LV-hGFAP: : BFP-T2A-Cre were used. For Ai14;Aldh1l1-CreERT2 reprogramming, LV-hGFAP:: Sox10-P2A-zsGreen, LV-hGFAP: : Olig2-P2A-zsGreen, LV-hGFAP: : Nkx6.2-P2A-zsGreen and control LV-hGFAP: : zsGreen were used. A multiplicity of infection (MOI) of 100 was used for all experiments. Virus-containing astrocyte media was placed on the cells and left overnight. Viral media was replaced with fresh astrocyte media one day post transduction (DPT). Three DPT, cells were switched to OPC differentiation media6 [DMEM/F12/Glutamax™ (Gibco Catalogue No. 11330032), 1X N2 (Gibco Catalogue No. 17502048), 1X B27 without vitamin A (Gibco Catalogue No. 17504044), 200 ng/ml SHH (R&D Systems Catalogue No. 464-SH), 20 ng/ml FGF (R&D Systems Catalogue No. 3139-FB), 4 ng/ml PDGF (Sigma Catalogue No. SRP3228)]. At 10 DPT, the cells were switched to OL differentiation media6 [DMEM/F12/Glutamax™, 1X N2, 1X B27 without vitamin A, 40 ng/ml T3 (T2877 Catalogue No. Sigma), 200 ng/ml SHH, 100 ng/ml Noggin (R&D Systems Catalogue No. 1967-NG), 10 UM cAMP (Sigma Catalogue No. A9501), 100 ng/ml IGF (R&D Systems Catalogue No. 791-MG), 10 ng/ml NT3 (Sigma Catalogue No. SRP6007)].

### Live Cell Analysis

Astrocytes isolated from Ai14;Aldh1l1-CreERT2 mice were plated in 96 well plates, transduced and imaged every hour from 7 to 12DPT using the Apotome™ live cell system (Zeiss). 25 z-stack tiled images per well were captured with brightfield as well as the 488 nm and 568 nm fluorescent wavelength. Images were stitched to create a continuous video for each well. At 12DPT, cells were fixed and stained for OLC markers to confirm fate. Each well was then re-imaged at this final timepoint and OLC+ reprogrammed cells were matched to the live cell video and retrospectively analyzed for starting cell morphology and fluorescent expression.

### Immunocytochemistry

Cells were fixed in 4% paraformaldehyde (PFA) (Sigma Catalogue No. P6148) for 20 minutes followed by three washes with 1×PBS. Cell membranes were permeabilized with 0.1% Triton-X-100 (Sigma Catalogue No. X100) for 10 minutes at room temperature, followed by three washes with 1×PBS, and then blocked with 5% milk for one hour at room temperature. Cells were incubated with primary antibodies in 1×PBS overnight at 4° C., washed three times with 1×PBS, and then incubated with secondary antibodies and DAPI (Sigma Catalogue No. D9542) in 1×PBS at room temperature for one hour. Following three final 1X PBS washes, coverslips were mounted on glass slides (Fisher Scientific Catalogue No. 125523) with Mowiol mounting solution (Sigma Catalogue No. 81381). For staining of membrane bound proteins (04, PDGFRa), no permeabilization step with Triton-X-100™ was performed.

Primary antibodies: mouse anti-SOX10 (RRID: AB_10844002, 1:250), rabbit anti-PDGFRa (RRID: AB_2892065, 1:500), mouse anti-04 (RRID: AB_357617, 1:1000) and rat anti-MBP RRID: AB_305869, 1:50).

Secondary antibodies: anti-mouse IgG 488 (Invitrogen Catalogue No. A32723) and 647 (Invitrogen Catalogue No. A32728), anti-mouse IgM heavy chain 488 (Invitrogen Catalogue No. A21042) and 647 (Invitrogen Catalogue No. A21238), anti-rabbit 488 IgG (Invitrogen Catalogue No. A11034) and 647 (Invitrogen Catalogue No. A32733), anti-rat IgG 488 (Invitrogen Catalogue No. A21208) and 647 (Invitrogen Catalogue No. A21247) all at 1:1000.

### Microscopy and Image Analysis

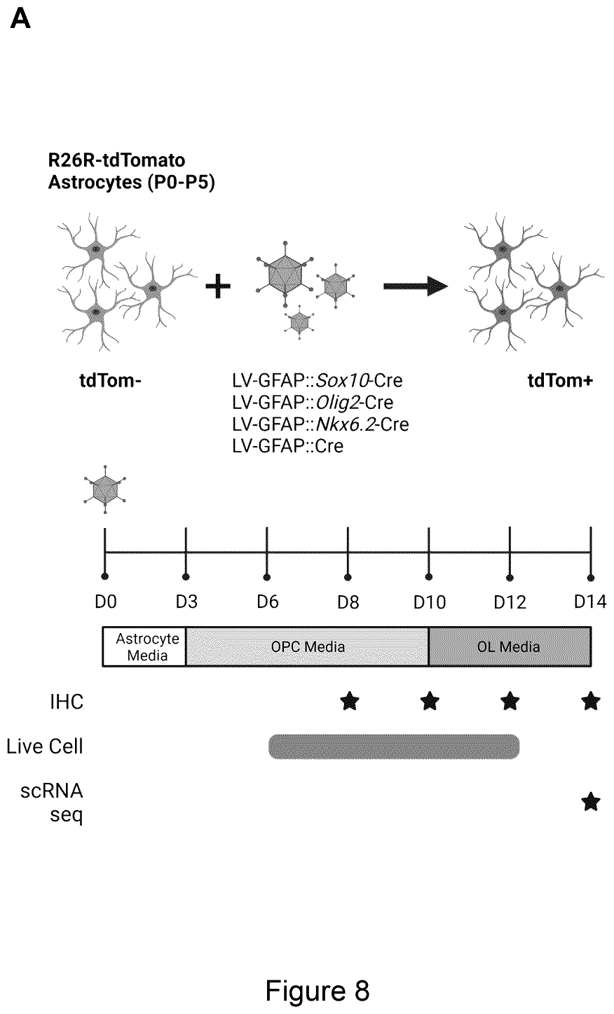

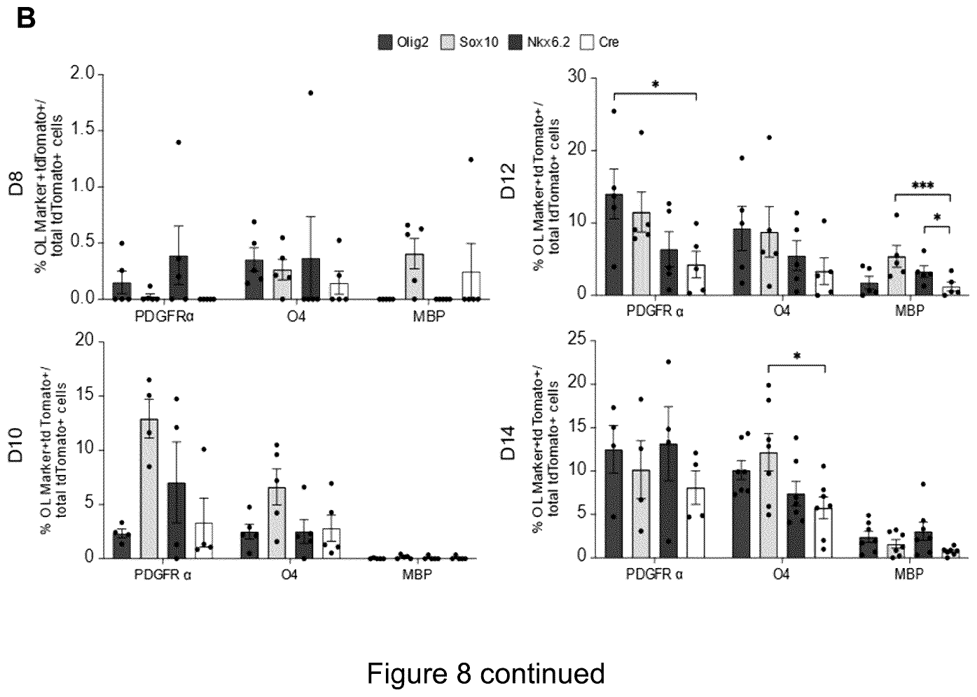

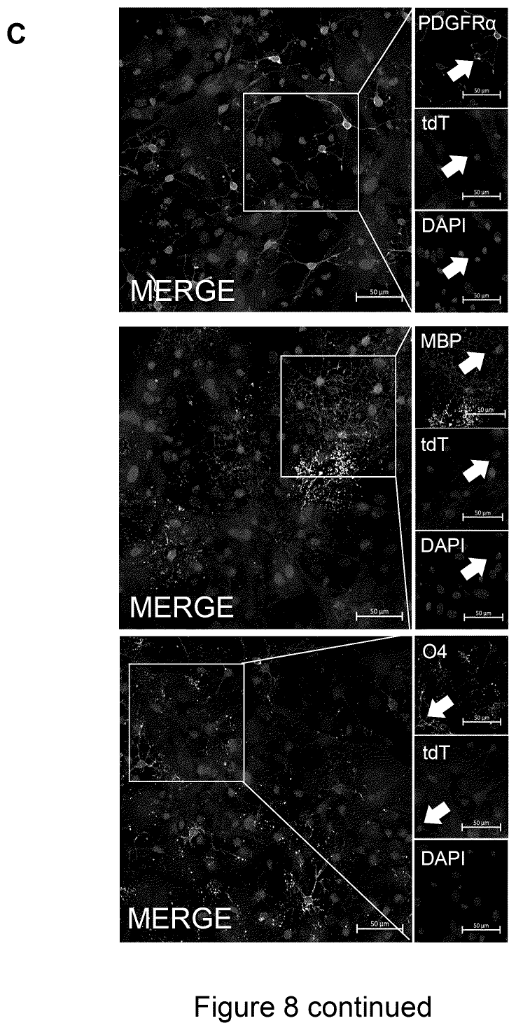

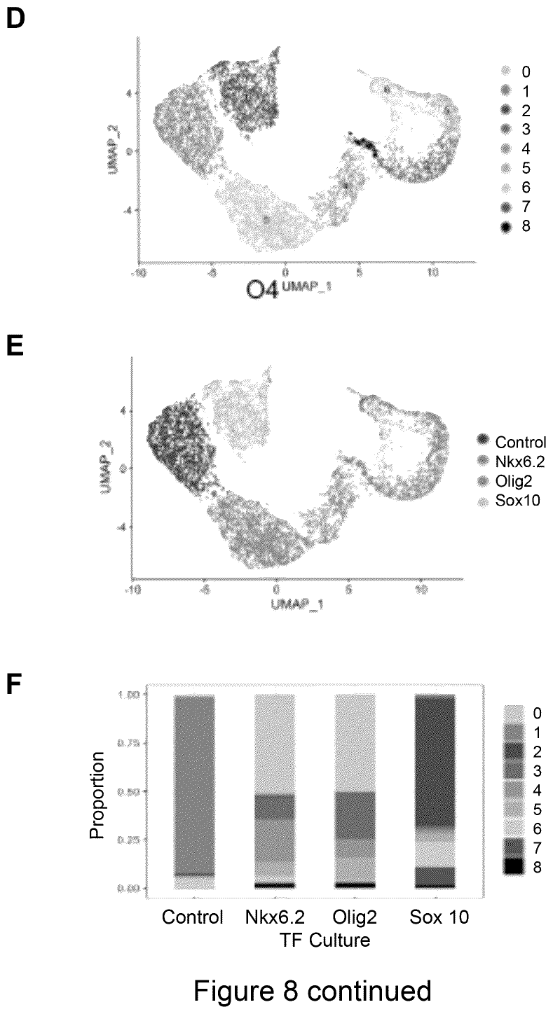

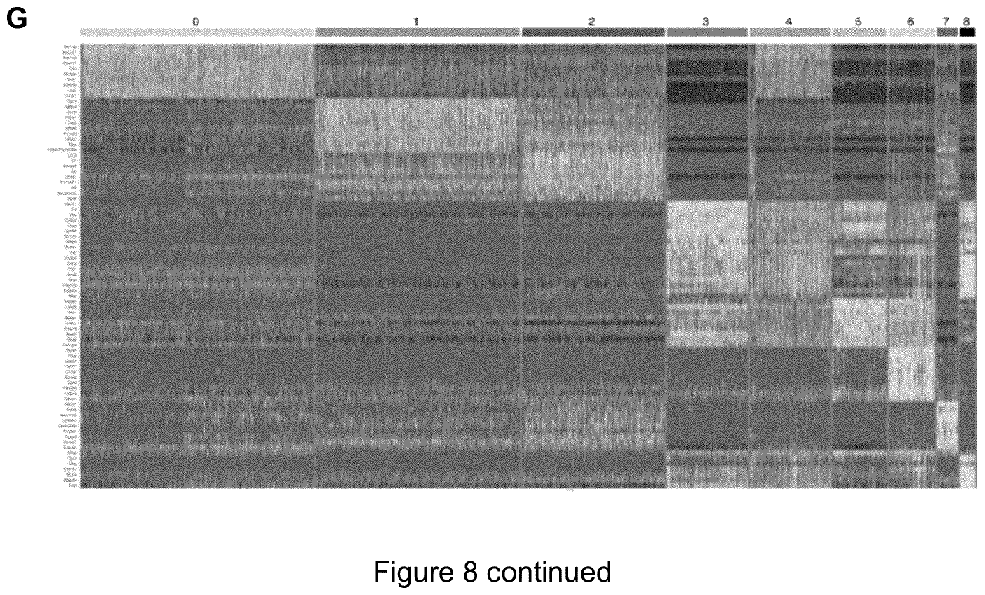

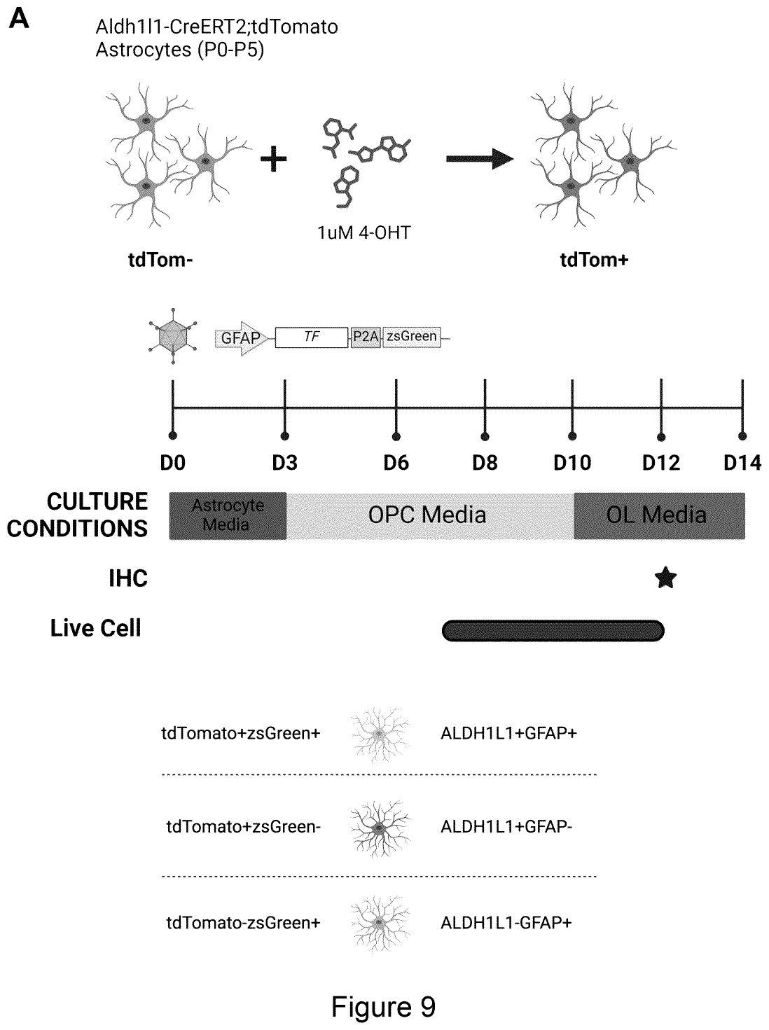

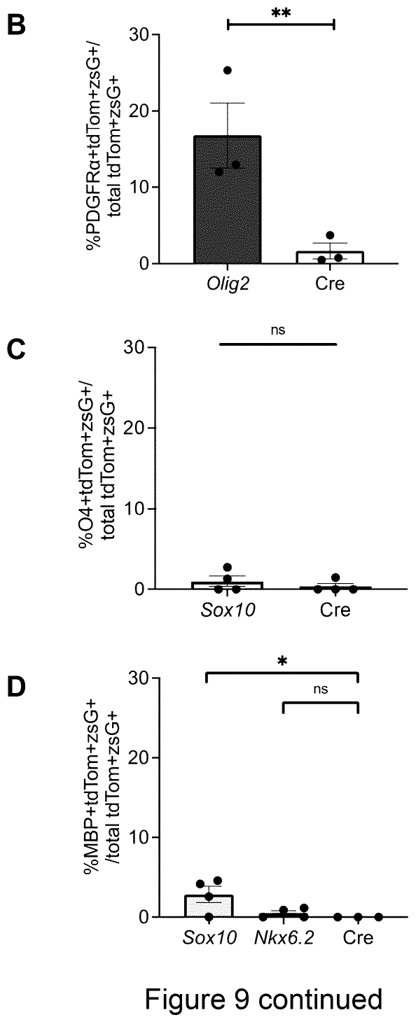

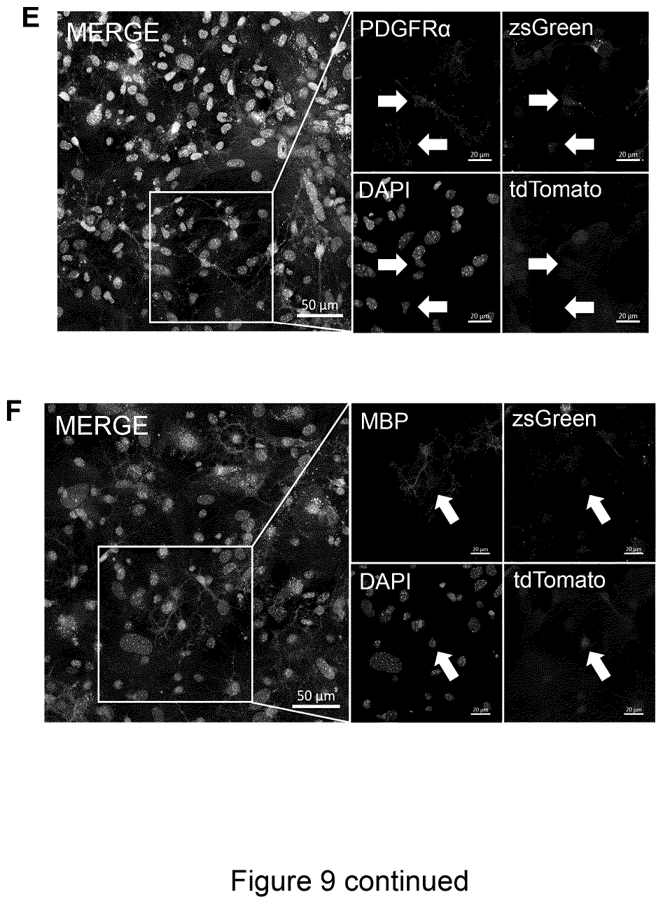

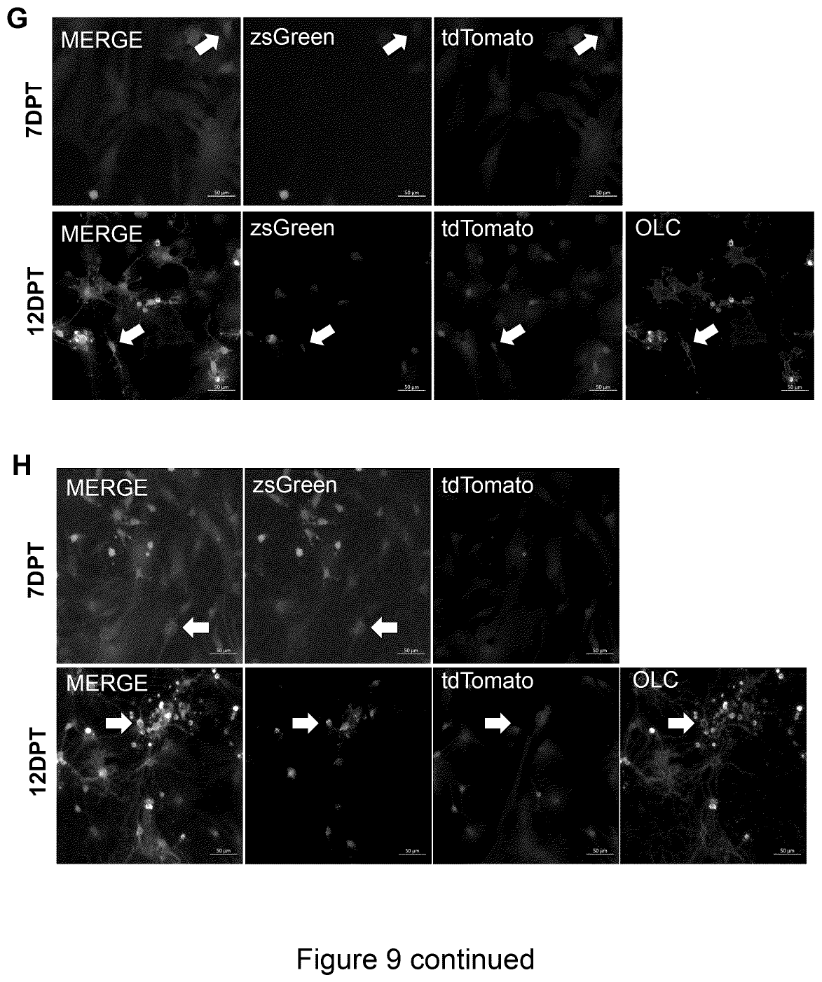

Fluorescent images for quantification were taken on an LSM 880™ Elyra Superresolution™ and LSM 900™ (Zeiss) using a 20× objective and Zen Blue™ software (Zeiss). Post-acquisition linear adjustments of brightness for all channels were made to micrographs using the Zen Blue™ software in FIG. 8C and FIG. 9E-H. For quantification, ten regions of interest were selected at random for each well. Images were analyzed using ImageJ™ software (National Institutes of Health, RRID: SCR_003070). Reprogramming efficiency was calculated as a measure of total OLC marker⁺reporter⁺DAPI⁺ cells over total reporter⁺DAPI⁺ cells.

scRNA-Seq Capture and Processing

At 14DPT, LV-hGFAP: : Sox10-P2A-Cre, LV-hGFAP: : Olig2-P2A-Cre, LV-hGFAP:: Nkx6.2-P2A-Cre, and control LV-hGFAP: : BFP-T2A-Cre cultures were processed using the BD Rhapsody System (BD Biosciences) and then sequenced. For single-cell isolation, an average of 9813.75 viable cells were captured in wells at cell load (Table 1 above). The BD Rhapsody scanner reported an average multiplet rate of 10.13% and an average number of wells with viable cells and a bead of 7081.5. Detailed metrics for each sample can be found in Table 1. Samples were down-sampled to 2500 cells and carried through and converted to cDNA using the BD Rhapsody™ WTA Reagent Kit (Becton Dickinson Canada, Catalogue No. 633802). Each cell was sequenced at approximately 100 million reads per cell (at least 2×150 bp paired-end reads) on a Novaseq™ (Donnelly Sequencing Centre, University of Toronto).

scRNA-Seq Analysis

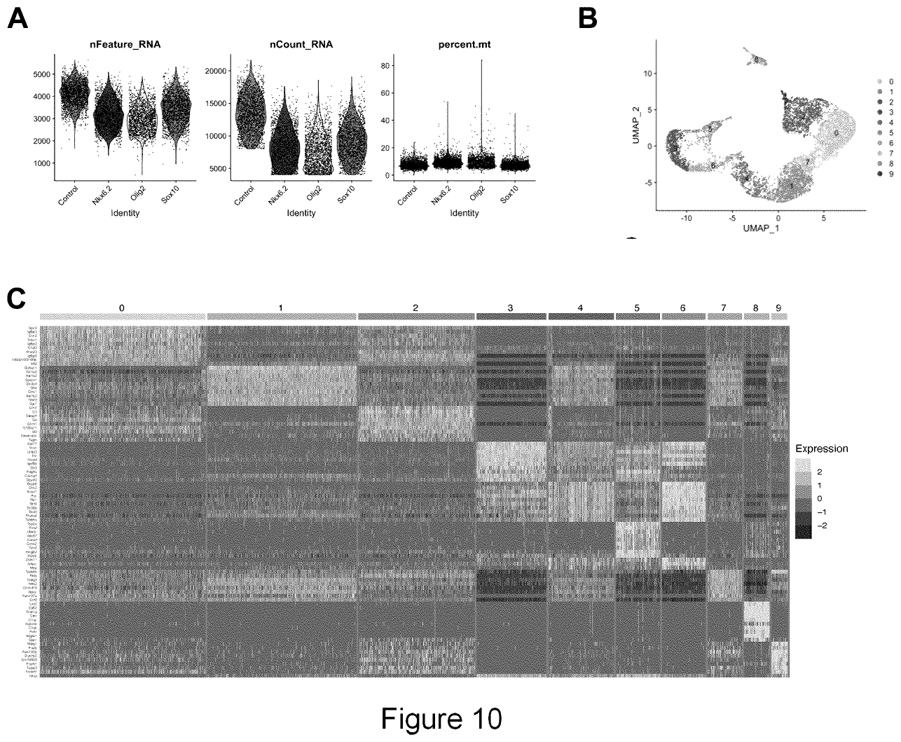

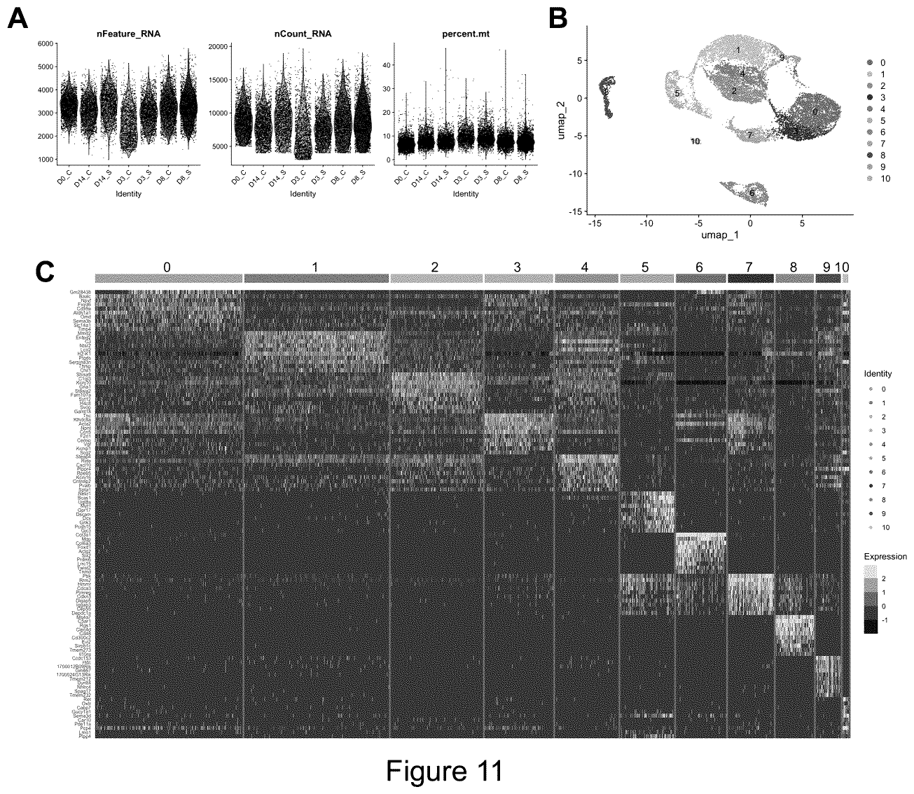

Fastq files were first demultiplexed with Kallisto (39) (RRID: SCR_016582) (v0.48.0) and Bustools (40) (RRID: SCR_018210) (V 0.41.0) using supplied whitelists (Data S1) with the -BDWTA option and aligning to GRCm38.96 with Cre sequence appended to the end. Bustools (40) was then used to generate gene count tables. Cells were plotted based upon UMI counts per barcode, thresholds were selected based on inflection point of UMI count per barcode plots. These thresholds produced read and gene count distributions that were comparable between all treatment groups (FIG. 10A, FIG. 11A). Gene count tables were made into S4 objects, scaled, normalized and dimensions reduced (PCA then UMAP) using the Seurat (41) package (RRID: SCR_016341) (v4.1). Clusters identified as microglia and as vascular and leptomeningeal cells (VLMCs) (FIG. 10B-C [cluster 8], FIG. 11B-C [cluster 6 and 8]) were removed prior to further analysis.

Gene markers for oligodendrocyte lineage cells were adopted from studies observing in vivo mouse oligodendrocyte lineage cells across several areas in young and mature CNS tissues (31). These markers were converted into percent expression of each UMAP cluster using the PercentageFeatureSet function from Seurat (41), with further gene resolution displayed by heatmaps created using ComplexHeatmap™ (42) (RRID: SCR_017270) (v12.13.1). An additional set of gene markers, demonstrating similar, but less resolved, conclusions was also used from an in vitro rat study looking at OLCs from the cortex (34). Stacked violin cluster dot plots were made with scCustomize (58) (RRID: SCR_024675) (v2.1.2). For pseudotime and trajectory analysis Slingshot (43) (RRID: SCR_017012) (v2.3.1) and Monocle3 (59-63) (RRID: SCR_018685) were used. For in silico Sox10 perturbation, CellOracle (64) (v0.14.0) was used. Plots were produced using ggplot2 (46) (RRID: SCR_014601) (v3.3.5) and figure generating scripts were run in R studio (47) (v4.2.0), with demultiplexing using Kallisto (39) and Bustools (40) run on a Compute Canada HPC cluster. All scripts used for processing of scRNA-seq data and for figure generation can be found at github.com/eyscott.

### Deep-Learning Analysis

Fatecode (65) was used to identify key genes for cellular transition. To optimize the autoencoder and subsequent classifier configuration, a grid search was conducted to systematically evaluate various combinations of hyperparameters. These included: latent layer size, number of nodes in the first and second layers of the autoencoder, classifier architecture, and type of activation function. The grid search aimed to identify the hyperparameters that minimized a combined reconstruction and classification loss function, signifying the optimal performance for our specific dataset. Perturbations on the latent space were performed and cell classifications as well as genes associated with each perturbation were obtained.

All scripts used for processing and for figure generation of Fatecode analysis can be found at https://github.com/MehrshadSD.

### Statistical Analysis

Percentage values were transformed using the arcsine square root transformation and assessed for normal distribution using the Shapiro-Wilks test. When distribution and variance were equal, a matched pairs one-way ANOVA ([Ai14: D8, D10, D12 04, D12 MBP], [Ai14;Aldh1l1-CreERT2: D12 MBP zsGreen⁺, D12 MBP tdTomato⁺) or one-way ANOVA ([Ai14: D14], [Ai14;Aldh1l1-CreERT2: D12 MBP zsGreen⁺,tdTomato⁺]), or paired t-test ([Ai14;Aldh1l1-CreERT2: D12 PDGFRa zsGreen⁺tdTomato⁺, D12 PDGFRa tdTomato⁺, 04 zsGreen⁺, 04 tdTomato⁺]) was performed to compare reprogramming efficiency of TF groups to a control group (Ai14: LV-GFAP: : Cre, Ai14;Aldh1l1-CreERT2: LV-GFAP: : zsGreen). When transformed values did not follow a Gaussian distribution, a Kruskal-Wallis test (Ai14: D12 PDGFRa, D14 PDGFRa) or Wilcoxon test (Ai14;Aldh1l1-CreERT2: PDGFRa zsGreen⁺, 04 zsGreen⁺tdTomato⁺) was performed to compare reprogramming efficiency to a control group (Ai14: LV-GFAP: : Cre, Ai14;Aldh1l1-CreERT2: LV-GFAP: : zsGreen). In both cases, Dunnett's post-hoc testing was performed to correct for multiple comparisons. Differences were considered significant at p<0.05. Values are presented as mean±SEM. The statistical software used for transformation, distribution, variance, ANOVA, Kruskal-Wallis, t-test, Wilcoxon test and Dunnett's analysis was GraphPad Prism version 9.0.1 (RRID: SCR_002798).

### Results

Different types of iOLCs are generated following expression of Olig2, Sox10 or Nkx6.2

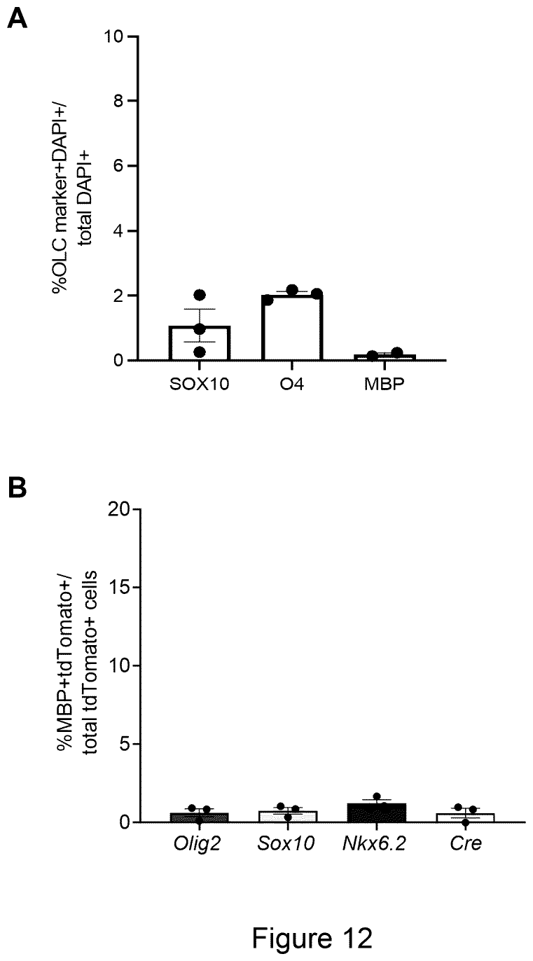

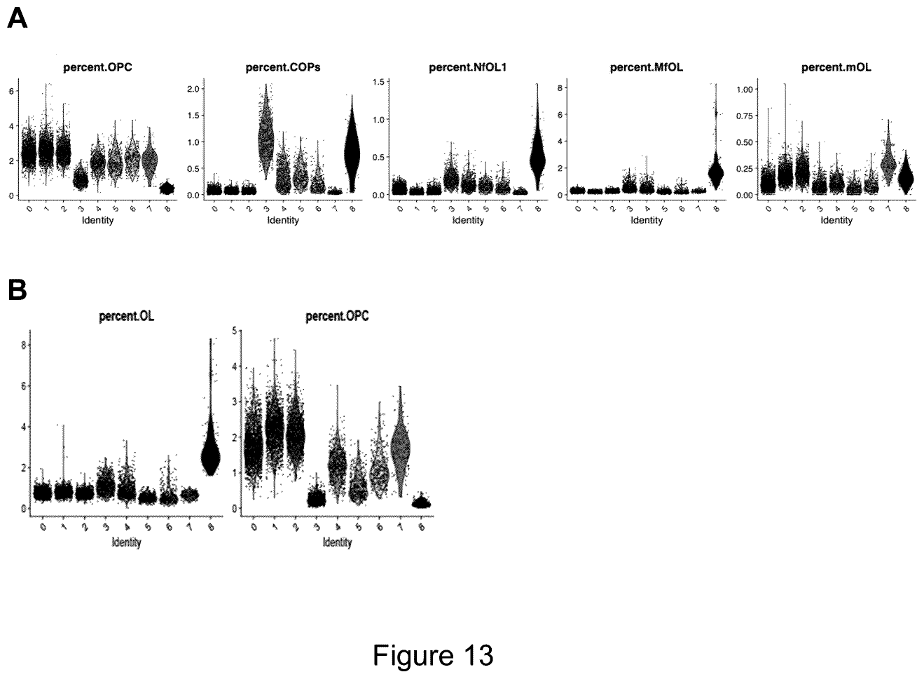

To investigate astrocyte to OLC conversion, postnatal, cortical astrocyte cultures from Ai14 mice were established. To understand the purity of our cultures, the numbers of contaminating OLCs were quantified. Quantification of SOX10+, 04+ and MBP⁺OLCs showed less than 2.5% in our cultures (FIG. 12A).

To examine the reprogramming potential of Olig2, Sox10, and Nkx6.2, Ai14 astrocytes were transduced with LV-GFAP: Olig2, LV-GFAP: : Sox10, LV-GFAP: Nkx6.2 or a control LV-GFAP: : Cre (FIG. 8A). When the numbers of tdTomato⁺ cells that co-expressed OPC (PDGFRa), COP (04) or OL (MBP) markers at 8 and 10 DPT were then quantifed, no differences were seen in any of the TF-transduced cultures compared to controls (FIG. 8B). By 12 DPT, Olig2-cultures showed an increase in the percentage of tdTomato⁺PDGFRa+ OPCs (p=0.0309, H-statistic: 7.514), whereas Sox10- and Nkx6.2-cultures showed an increase in tdTomato⁺MBP⁺OLs (Sox10: p=0.0004, Nkx6.2: p=0.0108, F statistic=13.28, degrees of freedom=3) (FIG. 8B-C). No differences were seen in the percentage of tdTomato⁺O4+ COPs in any condition at 12DPT (FIG. 8B). However, by 14DPT an increase in the percentage of tdTomato⁺O4+ COPs in Sox10-cultures was observed when compared to controls (p=0.0213, F statistic=3.512, degrees of freedom=3) (FIG. 8B-C). Finally, to understand whether a longer time in culture could increase the number of MBP⁺OLs, cells were cultured for an additional 8 days and the percent of tdTomato⁺MBP⁺ cells at 22 DPT was analyzed. No differences were seen in tdTomato⁺MBP⁺OLs in any condition compared to controls (FIG. 12B). Altogether, these findings suggest that Olig2, Sox10 and Nkx6.2 increase different types of iOLCs at different times relative to controls in Ai14 astrocyte cultures.

Canonical OLC Cluster Found in TF-Treated Cells Following scRNA-Seq

To further characterize Olig2-, Sox10-, Nkx6.2- and control cultures, scRNA-seq was performed at 14DPT (FIG. 8A). Clustering analysis showed the appearance of nine clusters (FIG. 8D). Proportion analysis was then used to identify clusters that were unique to TF-treated samples. Clusters 3 and 8 were predominantly comprised of cells from TF-induced cultures (FIG. 8E-F). The top 10 genes marking each cluster in FIG. 8D are highlighted in FIG. 8G. Canonical OLC genes such as Mbp, Plp1, Bcas1 and Cldn11 were expressed in clusters 3 and 8 (FIG. 8G), further suggesting that the increase in iOLCs is TF-dependent.

The molecular profiles of clusters 3 and 8 were compared to established OL lineage datasets. When using OLC specific annotations of mouse fetal and adult OLCs derived from Marques et al (33) to bin the data it was found that cluster 3 was characterized by COP signatures, while cluster 8 was characterized by COP, newly formed oligodendrocyte (nfOL) and myelin forming oligodendrocyte (mfOL) signatures (FIG. 13A). When using OLC annotations of in vitro OLCs derived from Dugas et al (34) to bin the data, it was again observed that cluster 8 was characterized by OL signatures, but cluster 3 was not. (FIG. 13B). Altogether, these findings further support the observation of increased OLC generation in TF-induced Ai14 cultures (FIG. 8B).

Bonafide Astrocyte to OLC Conversion is Confirmed with Astrocyte Fate Mapping.

Contaminating OLCs were previously observed in the cultures and the presence of tdTomato⁺OLC marker⁺ cells was previously observed in Cre controls. Therefore, to understand the origin of these newly generated OLCs, astrocyte fate mapping was performed using Ai14;Aldh1l1-CreERT2 cultures, the current gold standard in the field (57). Prior to transduction, post-natal cortical Ai14;Aldh1l1-CreERT2 astrocyte cultures were treated with 1 uM 4-OHT to permanently label all Aldh1l1 expressing cells with tdTomato (FIG. 9A). Cultures were transduced with LV-GFAP: : Sox10-zsGreen, LV-GFAP: : Olig2-zsGreen, LV-GFAP:: Nkx6.2-zsGreen or a LV-GFAP: : zsGreen control (FIG. 9A). Any OLCs that were directly converted by a TF from these Aldh1l1 expressing astrocytes would therefore express zsGreen and tdTomato (FIG. 9A). To understand whether the OPCs generated from Olig2 overexpression were the product of astrocyte reprogramming, the percentage of PDGFRa⁺zsGreen⁺tdTomato⁺ cells at was quantified 12DPT. An increase in PDGFRa⁺zsGreen⁺tdTomato⁺OPCs was observed compared to controls, confirming the astrocytic origin of iOPCs (FIG. 9B,E). To assess the origin of Sox10-induced COPs, the percentage of O4+zsGreenttdTomato⁺ cells was quantified at 14DPT. No increase was observed in Sox10 cultures compared to controls (FIG. 9C). Finally, to determine the origin of OLs generated by Sox10 or Nkx6.2, the percentage of MBP⁺zsGreen⁺tdTomato⁺OLs was quantified at 12DPT. An increase of MBP⁺zsGreenttdTomato⁺OLs generated by Sox10 was observed, but not from Nkx6.2 (Figure D,F). This confirms that Olig2 and Sox10 reprogram astrocytes to iOPCs and iOLs, respectively but that the Nkx6.2-derived OLs and Sox10-derived COPs we previously observed (FIG. 8B) may not have been the result of Aldh1l1 positive astrocyte conversion under the conditions used (but may have been the result of Aldh1lq negative astrocyte conversion).

To further confirm these findings, a live cell imaging experiment was performed. Ai14;Aldh1l1-CreERT2 Sox10-treated and control cultures were imaged from 7DPT to 12DPT then fixed and stained for OLC markers. tdTomato⁺zsGreen⁺OLC marker⁺ cells were then tracked retrospectively to confirm their origin from a Aldh1l1-tdTomato⁺ cell. tdTomato⁺zsGreen⁺OLC marker⁺OLs could be tracked back to a tdTomato⁺ cell with a characteristic astrocyte morphology (FIG. 9G). Taken together, these findings further confirm bonafide astrocyte to iOLC conversion.

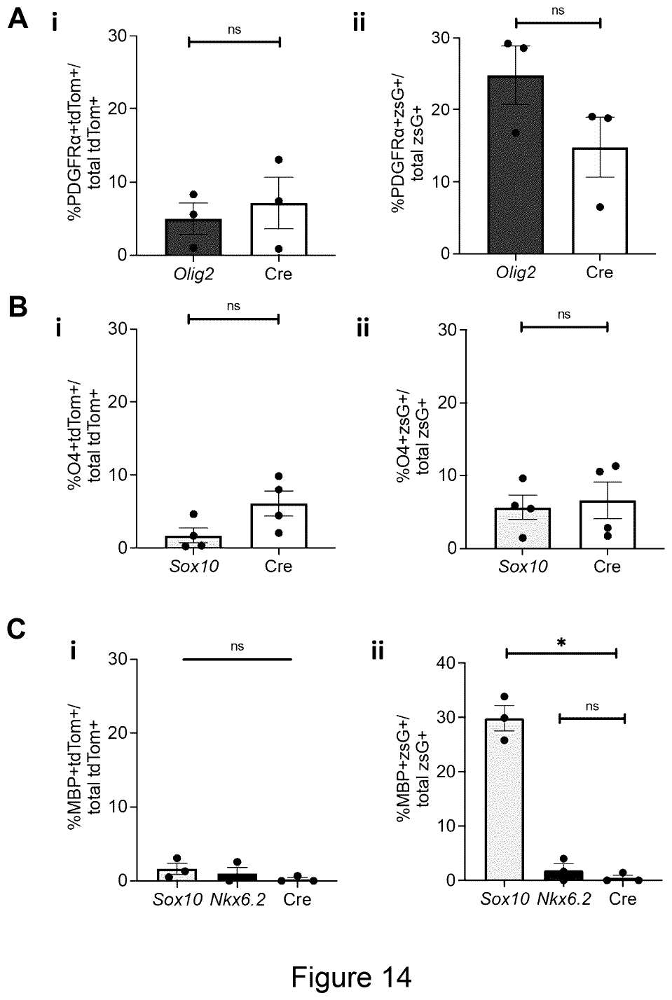

The numbers of Aldh1l1⁺ non-transduced (tdTomato⁺ only) cells and Aldh1l1neg virally transduced (zsGreen⁺ only) cells were also examined (FIG. 14). No difference in the percentage of OLC marker⁺tdTomato⁺zsGreenneg was seen between TF-treated and control cultures (FIG. 14). However, when zsGreen⁺tdTomato^{neg} cells were examined, a surprising increase in the percentage of MBP⁺zsGreen⁺tdTomato^{neg} OLs was observed at 12DPT in Sox10-treated cells compared to controls (FIG. 14C). When the live cell imaging experiment was analyzed to understand the origin of these cells, examples of tdTomato^{neg}zsGreen⁺OLCmarker⁺ cells were found that arose from a tdTomato^{neg} cell with an astrocyte morphology (FIG. 9H), suggesting conversion of other astrocyte-like Aldh111^{neg} cells, which further indicated the existence of an astrocyte or astrocyte-like population that expresses low Aldh1l1 but has high reprogramming efficiency.

Characterization of Sox10-Mediated DLR Using scRNA-Seq

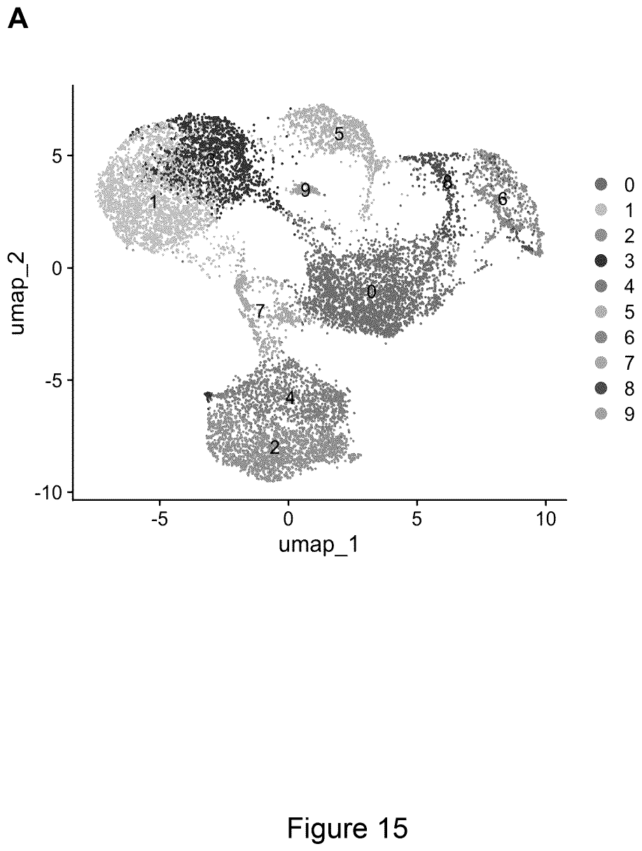

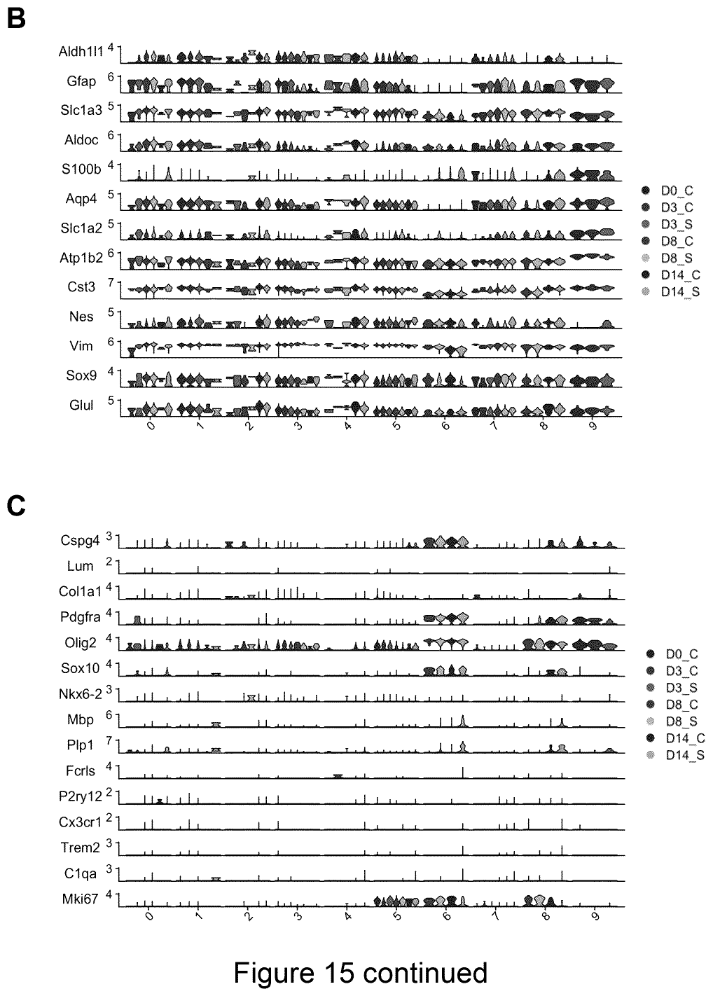

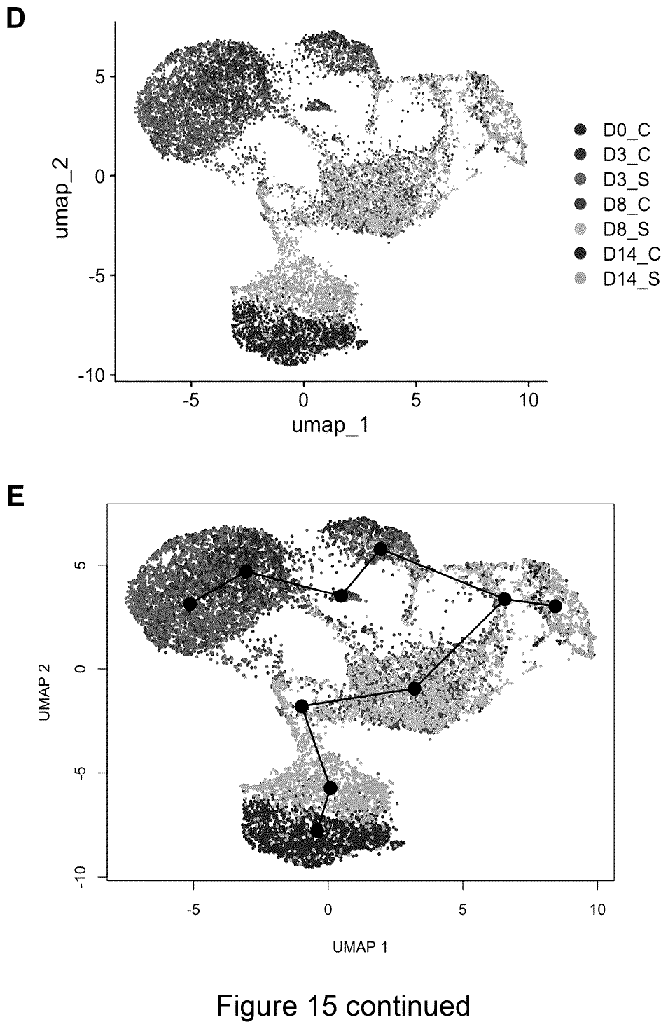

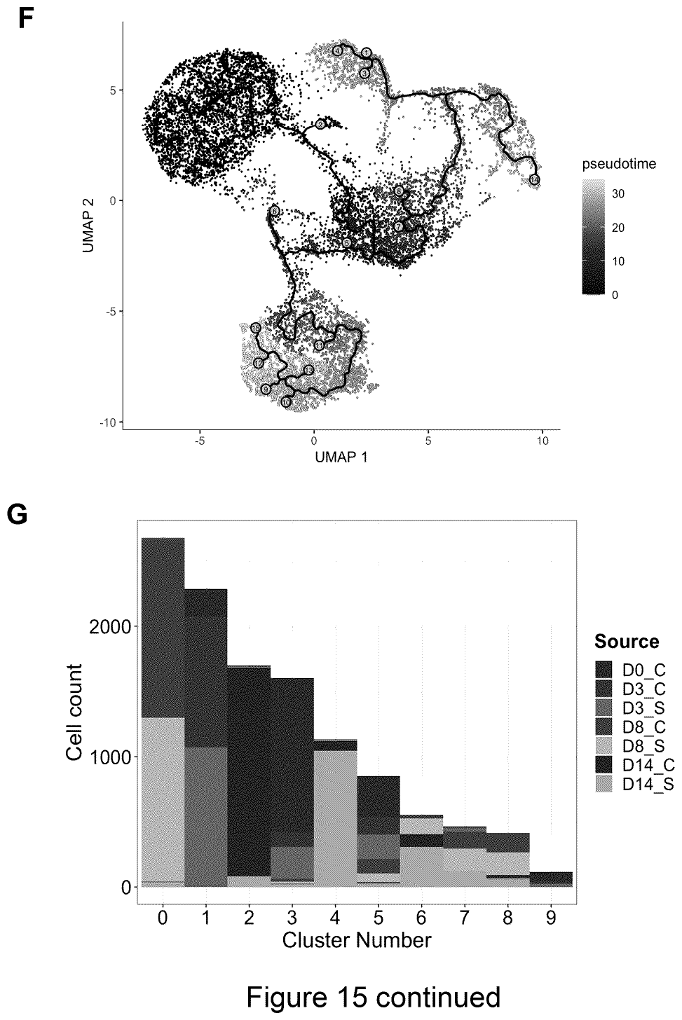

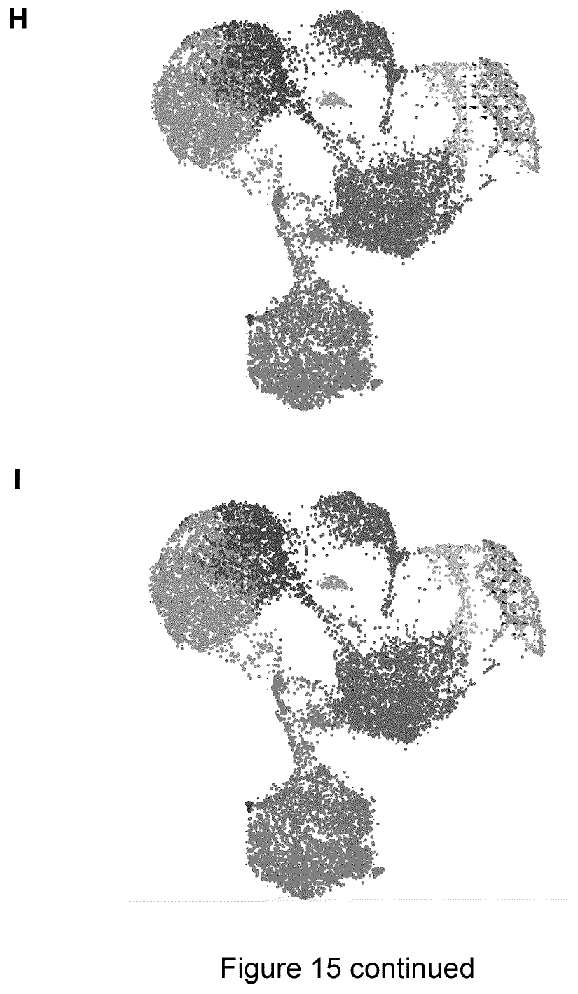

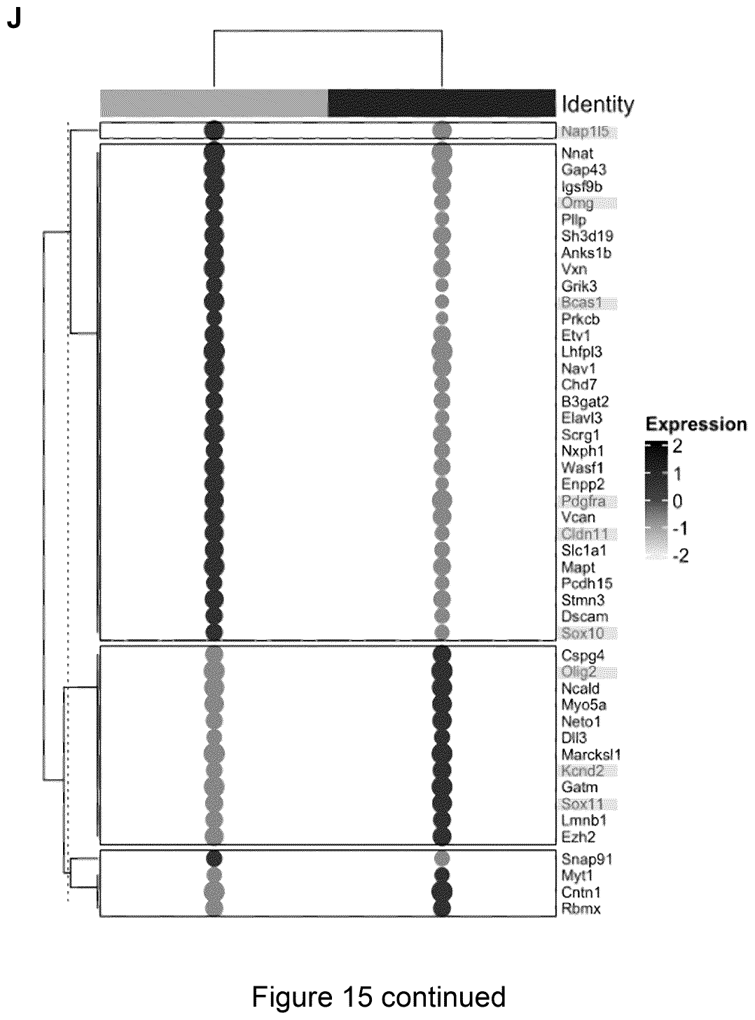

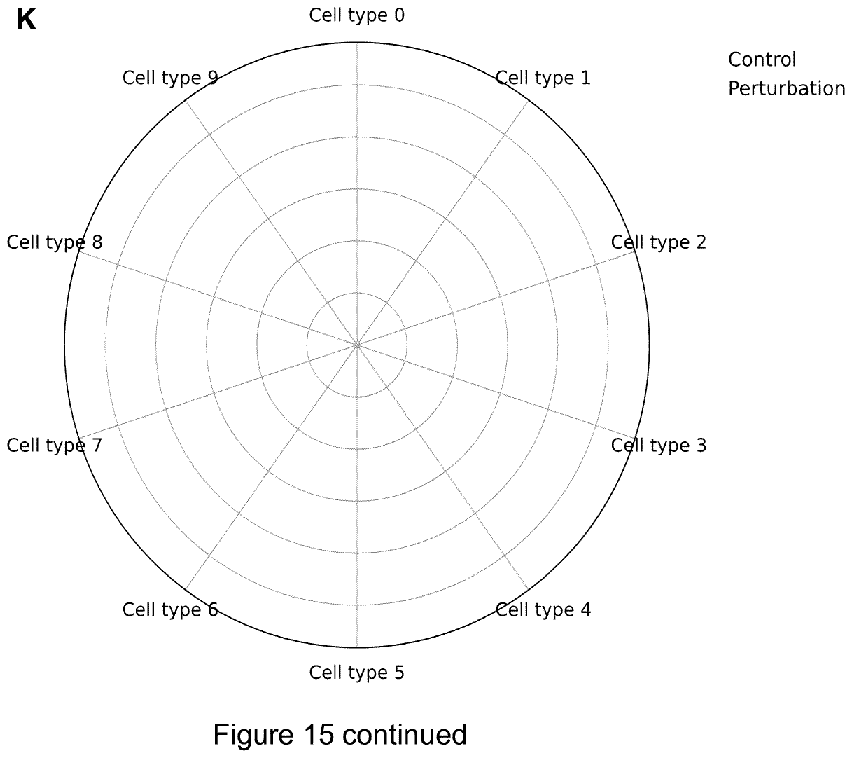

To better resolve the process of reprogramming, scRNA-sequencing was performed at three time points (do prior to transduction, 3DPT, and 8DPT) on Cre-control and Sox10-treated cultures. For analysis, cells from DO, 3DPT, 8DPT and 14DPT were combined (FIG. 11B). Cells with microglia and VLMC markers were excluded (FIG. 11C). Clustering analysis of the remaining cells showed the appearance of 10 distinct clusters (FIG. 15A). These clusters were characterized based on expression of genes associated with astrocytes, oligodendrocytes, and NG2 cells, as well as proliferation markers (FIG. 15B-C).

First, the molecular identities of cells in the starting cultures were examined. DO cells were found in clusters 3, 5 and 9 and showed expression of canonical astrocyte markers, including Slc1a3, Aqp4, Atp1b2, Glul and Gfap (FIG. 15B, D). Of interest, clusters 3 and 5 showed expression of Aldh1l1, but this gene was absent in cluster 9 (FIG. 15B). This discrepancy in Aldh1l1 expression, may explain the conversion of tdTomato^{neg} cells seen in the fate mapping experiments (FIG. 14). Altogether, this suggests that early postnatal cultures are heterogeneous and comprised of 3 distinct astrocyte-like populations (clusters) and provides additional support for the existence of an Aldh1l1 negative astrocyte/astrocyte-like population that can be reprogrammed.

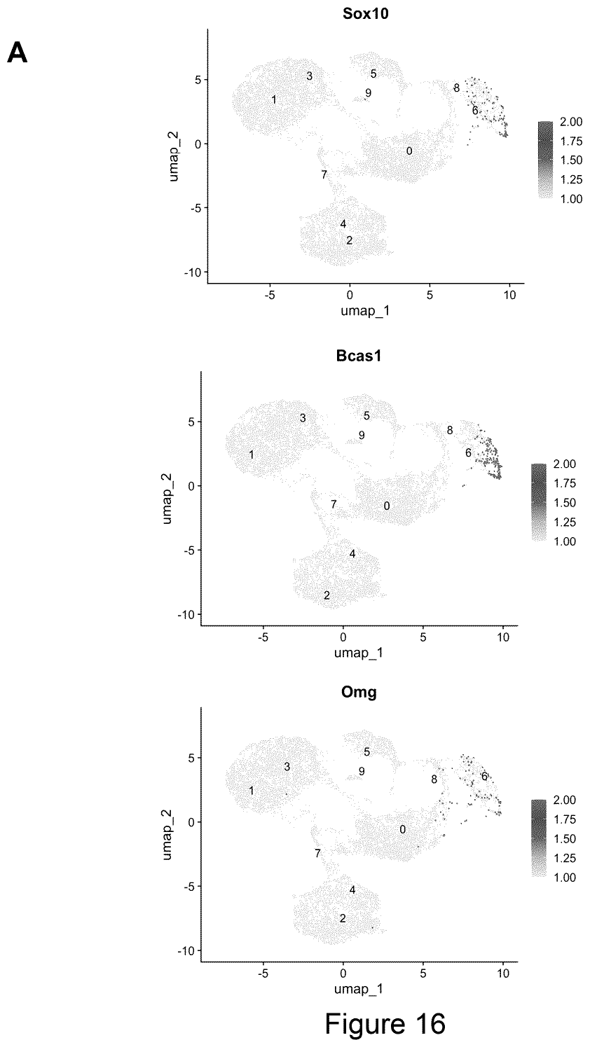

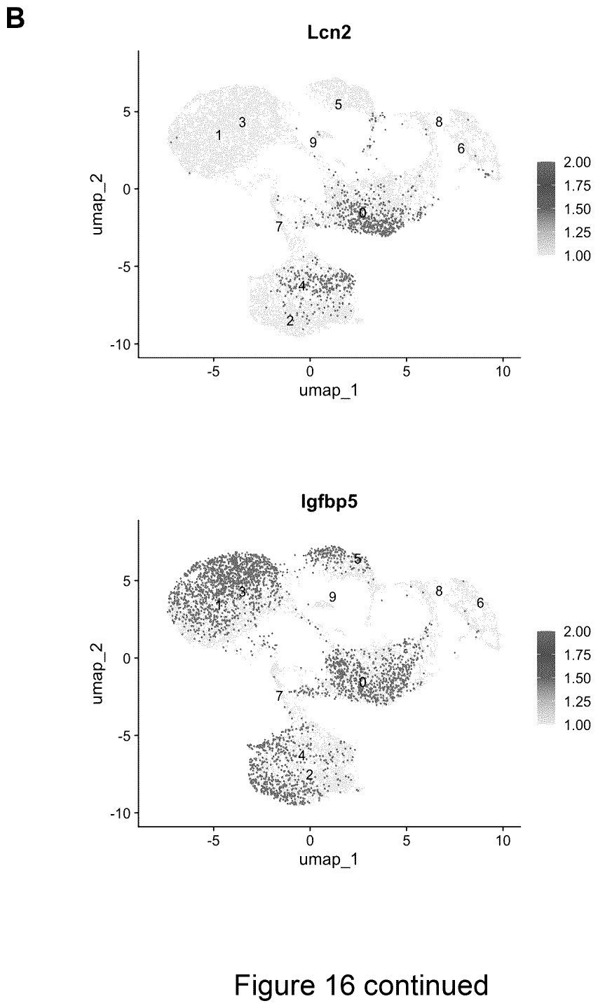

To understand the cell fate transitions that occur over the reprogramming timecourse, trajectory analysis was performed with Slingshot (43) (FIG. 15E) and Monocle3 (59-63) (FIG. 15F). Slingshot and Monocle3 both reconstructed trajectories with clusters 6 and 4 as terminal branches (FIG. 15E-F). The origin of cells comprising each cluster was then examined. As expected, the clusters formulating earlier roots of the Monocle3 branches (clusters 0, 1, 3) were primarily comprised of DO, 3DPT, and 8DPT timepoints (FIG. 15G). Clusters 6 and 4, were predominantly comprised of 14DPT samples, and in particular, cells from Sox10-treated samples (D14_S) (FIG. 15G). Analysis of the differentially expressed genes (DEGs) at these branches showed that cluster 6 and branch 14 of the Monocle3 trajectory were enriched in the OLC genes Sox10 (11-13,56) (p- and q-value=0), Bcas1 (66) (p- and q-value=0), and Omg (67,68) (p- and q-value=0 (FIG. 16A)). In contrast, cluster 4 and the Monocle trajectory branch 11 showed expression of the genes Lcn2 (p- and q-value=0) and Igfbp5 (p- and q-value=0), (FIG. 16B) previously shown to be expressed in reactive astrocytes (26,69,70). Altogether, these analyses suggest that the trajectory to branch 14/cluster 6 represents the path of astrocyte to OLC conversion.

To determine how Sox10 would influence the gene regulatory network of cells in clusters 6 and 4, in silico perturbation of Sox10 was performed using CellOracle (64), in silico knock out (KO) of Sox10, predicted a large shift away from a cluster 6 identity and little change away from the identity of cluster 4 (FIG. 15H). In agreement, in silico knock in (KI) of Sox10 showed a large shift towards a cluster 6 identify and little change towards a cluster 4 identity (FIG. 15I). This suggests that leaf 14 and cluster 6 represent cells with a gene regulatory network most affected by Sox10.

As cluster 6 was comprised of both Sox10-treated and control cells, a differential gene expression analysis of 14DPT control and Sox10-treated cells within this cluster was performed. 175 DEGs were found, with the top genes (expressed in at least 50% of Cluster 6, Sox10-treated cells and with a difference of at least 0.5 pct between control and Sox10-treated cultures) clustered and provided in a dotplot (FIG. 15J). This analysis highlighted that genes including Omg, Bcas1, Pdgfra, Cldn11 (71), and Sox10 are enriched in Sox10-treated cells compared to control cells, suggesting that Sox10-treated cells are more representative of OLCs than the control cells exposed to OPC and OL media alone.

### Understanding the Genetic Drivers of Astrocyte to OLC DLR

The present analysis showed that Sox10 was important for determining the OLC identity of cluster 6. Sox10 was specifically chosen based on its role in OLC fate specification in development (11,12). To understand whether there might be other [better] candidate genes that would promote an OLC identity, an unbiased, deep learning perturbation model, Fatecode (65), was used to predict genes that would allow cells to shift from a cluster 4 identity (astrocytes at 14DPT, not fully reprogrammed) to a cluster 6 identity (OLCs at 14DPT, end state of DLR). Following training of the Fatecode model on the present Sox10 and control treated DLR dataset, perturbation on node 16 of the latent layer was identified as one which increased the number of cells in cluster 6 whilst simultaneously reducing the number of cells in clusters 0, 2, 4, 5 and 7. (FIG. 15K). Given that the total number of cells in the dataset does not change, this suggests that the perturbation on node 16 of the latent layer was pushing cells that did not reprogram (astrocytes at 8 and 14DPT) towards a reprogrammed (OLCs at 14DPT) fate. To identify the genes driving this shift, the absolute value of gene expression changes was ranked to obtain a list of 3000 genes involved in this perturbation, with the top 40 genes listed in Table 2. Strikingly, it was observed that Sox10 was ranked as the ninth most correlated gene and the top TF driving this shift (Table 2). This supports finding that Sox10 drives astrocyte to OLC DLR. Additionally, we observed genes important for myelination (Plp1, Mbp) (72), as well as other TFs previously identified with OLC differentiation and myelination (Sox3 (73), Klf9 (74)) as integral to this shift. Taken together, these findings further support the importance of Sox10 in reprogramming astrocytes to OLCs and identify additional genes and TFs that may be candidates for future testing for astrocyte to OLC reprogramming. However, these findings are theoretical only and do not provide sufficient data to support a prediction that such genes and TFs will be useful in astrocyte to OLC reprogramming. Given the complexities of reprogramming, in vitro experiments would be required to evaluate such a use and provide an indication of feasibility of use of these candidate.

TABLE 2
Top 40 genes associated with Fatecode perturbation
on node 16 of the latent layer

| Rank | Gene Name |
|---|---|
| 1 | Plp1 |
| 2 | Anks1b |
| 3 | Pllp |
| 4 | Acot1 |
| 5 | Armh4 |
| 6 | Mbp |
| 7 | Tdg |
| 8 | Lrrtm2 |
| 9 | Sox10 |
| 10 | Chd7 |
| 11 | Bzw2 |
| 12 | B3gat2 |
| 13 | Insig1 |
| 14 | S100b |
| 15 | Idh2 |
| 16 | Kcnd2 |
| 17 | Bex2 |
| 18 | Sall3 |
| 19 | Chl1 |
| 20 | Pals2 |
| 21 | Neto1 |
| 22 | Sox12 |
| 23 | Wasf1 |
| 24 | Sox3 |
| 25 | Plppr5 |
| 26 | Spry2 |
| 27 | Fnbp1l |
| 28 | Grm5 |
| 29 | Ppfibp1 |
| 30 | Camsap2 |
| 31 | Ppp1r16b |
| 32 | Ptpre |
| 33 | Serinc5 |
| 34 | B3gat1 |
| 35 | Lrrc17 |
| 36 | Elovl4 |
| 37 | Mmp15 |
| 38 | Trib2 |
| 39 | Klf9 |
| 40 | Hipk1 |

### DISCUSSION

In this Example, astrocyte to OLC conversion with Sox10 or Olig2 has been shown using a battery of experimental tools, including astrocyte fate mapping, live cell imaging, a scRNA-seq timecourse and unbiased deep learning. While previous studies demonstrated the generation of iOLCs from different types of somatic cells using combinations of Sox10, Olig2 and Nkx6.2 (6,7, 36), this is the first study comparing the individual reprogramming ability of each these TFs in astrocytes. Using Aldh1l1-based fate-mapping, it was found that that Sox10 converts Aldh1l1+astrocytes to MBP⁺ oligodendrocytes, whereas Olig2 converts Aldh1l1+astrocytes to PDGFRa+ OPCs.

In a previous study, Khanghahi et al. investigated Sox10 mediated astrocyte to OLC conversion. Ectopic expression of Sox10 in astrocytes in vitro led to an increase in OPCs at 21DPT (20), a different OLC type and longer time to conversion than observed by the present inventors. Although similar experimental designs were used in both studies (cortical P3-P5 astrocytes cultured in OPC media in Khanghahi et al. versus P0-P5 cortical astrocytes cultured in OPC followed by OL media in our study), these discrepancies may be due to the use of different viral delivery strategies and metrics of reprogramming. In the present Example, lentiviral delivery of Sox10-P2A-zsGreen under the control of the long (2178 bp) hGFAP promoter (76) was used. In addition, Ai14;Aldh1l1-CreERT2 mice were used and only the iOLCs with an astrocyte origin were quantified (based on tdTomato⁺OLC marker⁺ expression). In contrast, in the Khanghahi et al. study, the authors used a SFFV promoter to deliver Sox10-IRES-GFP and reported the number of GFP+iOLCs in their Sox10-transduced cultures. Without an astrocyte specific promoter and stringent lineage tracking it is not possible to conclude that their iOPCs were the result of astrocyte conversion whatsoever. The non-specific delivery strategy likely instead hit a contaminating, perhaps more distantly related cell that would need more time to convert.

In this regard, a recent study suggested that the conversion reported in studies of in vivo astrocyte to neuron DLR (3,77,78) were not true reprogramming, but rather the result of erroneous labelling of endogenous neurons due to technical confounds (57). As a result, the DLR community has advocated for the stringent validation of DLR paradigms. In this example, astrocyte to iOLC conversion was first examined in Ai14 cells. This enabled the permanent labeling of transduced cells and therefore, the tracking of those cells through the DLR timecourse. To then validate the findings, Aldh1l1-astrocyte fate mapping was used, which highlighted a lack of conversion with Nkx6.2.

When performing the lineage tracing experiments, a subpopulation of cells (Aldh1l1-tdTomato^{neg}zsGreen⁺OLCmarker⁺) was also discovered that was converted by Sox10 at a high reprogramming efficiency (FIG. 14C). scRNA-seq analysis of cells prior to conversion also showed a cluster with low Aldh11 expression compared to the other astrocyte clusters, but with similar levels of Gfap expression (FIG. 15B), which could explain its transduction by the LV-GFAP:: Sox10. These Aldh111^{lo}Gfap^{hi} cells were predominantly characterized by mature astrocyte markers but also showed expression of genes found in NG2 glia (Cspg, Pdgfra) (FIG. 15B-C). Curiously, this population did not show expression of proliferation marker Mki67 (FIG. 15C), in contrast to previous studies showing that astrocyte-like NG2 glia are proliferative (79, 80). Of interest, one study reported high co-expression of Gfap Cspg4 and Pdgfra in early postnatal astrocytes (81). This leads to the intriguing idea that these cells represent a population of Aldh111^{lo} expressing astrocytes, which could preclude our understanding of the true extent of astrocyte reprogramming possible when using Aldh1l1-fate-mapping. The presence of these Aldh11/° cells shows that astrocyte cultures are heterogeneous and that different types of astrocytes or astrocyte-like cells may be suitable targets for DLR.

Conversion of Aldh1l1^{Pos} astrocytes to PDGFRattdTomato⁺zsGreen⁺ iOPCs using Olig2 occurred at a relatively low rate, with an average conversion efficiency of 16.75% cells. This conversion was even lower for the generation of mature MBP⁺tdTomato⁺zsGreen⁺OLs, with an average conversion efficiency of 2.83%. This may suggest that although Sox10 generates more mature OLCs, it can only do this in a select number of ‘elite’ donor cells. Alternatively, the absence of a substrate to myelinate in the cultures used in this Example may preclude the true reprogramming ability of Sox10. OL survival in vitro and in vivo has been shown to be dependent on the presence of axons (82). Furthermore, previous studies have shown that mRNA expression of myelin genes is increased when OLs are cultured in the presence of neurons (83), and differentiation of OPCs can be induced with bead or nanofiber scaffolding (84,85). However, the present Example demonstrated successful DLR for production of induced oligodendrocyte lineage cells (iOLCs) from donor astrocytes using a vector encoding a TF, where the single TF is Olig2 or Sox10, and the nucleic acid encoding the single TF is operably linked to an astrocyte-specific promoter sequence.

### REFERENCES

1. N. Heins, P. Malatesta, F. Cecconi, M. Nakafuku, K. L. Tucker, M. A. Hack, P. Chapouton, Y. A. Barde, M. Gootz, Glial cells generate neurons: the role of the transcription factor Pax6. Nat. Neurosci. 5, 308-315 (2002).
2. U. Pfisterer, A. Kirkeby, O. Torper, J. Wood, J. Nelander, A. Dufour, A. Björklund, O. Lindvall, J. Jakobsson, M. Parmar, Direct conversion of human fibroblasts to dopaminergic neurons. Proc. Natl. Acad. Sci. U.S.A 108, 10343-10348 (2011).
3. Z. Guo, L. Zhang, Z. Wu, Y. Chen, F. Wang, G. Chen, In vivo direct reprogramming of reactive glial cells into functional neurons after brain injury and in an Alzheimer's disease model. Cell Stem Cell. 14, 188-202 (2014).
4. S. A. Back, B. H. Han, N. L. Luo, C. A. Chricton, S. Xanthoudakis, J. Tam, K. L. Arvin, D. M. Holtzman, Selective vulnerability of late oligodendrocyte progenitors to hypoxia-ischemia. J. Neurosci. 22, 455-463 (2002).
5. T. Kuhlmann, V. Miron, Q. Cuo, C. Wegner, J. Antel, W. Bru«C k, T. Kuhlmann, Differentiation block of oligodendroglial progenitor cells as a cause for remyelination failure in chronic multiple sclerosis (2008), doi: 10.1093/brain/awn096.
6. F. J. Najm, A. M. Lager, A. Zaremba, K. Wyatt, A. V. Caprariello, D. C. Factor, R. T. Karl, T. Maeda, R. H. Miller, P. J. Tesar, Transcription factor-mediated reprogramming of fibroblasts to expandable, myelinogenic oligodendrocyte progenitor cells. Nat. Biotechnol. 31, 426-433 (2013).
7. K. P. Kim, C. Li, D. Bunina, H. W. Jeong, J. Ghelman, J. Yoon, B. Shin, H. Park, D. W. Han, J. B. Zaugg, J. Kim, T. Kuhlmann, R. H. Adams, K. M. Noh, S. A. Goldman, H. R. Schöler, Donor cell memory confers a metastable state of directly converted cells. Cell Stem Cell. 28, 1291-1306.e10 (2021).
8. Q. Zhou, S. Wang, D. J. Anderson, Identification of a novel family of oligodendrocyte lineage-specific basic helix-loop-helix transcription factors. Neuron. 25, 331-343 (2000).
9. C. A. G. Marshall, B. G. Novitch, J. E. Goldman, Olig2 directs astrocyte and oligodendrocyte formation in postnatal subventricular zone cells. J. Neurosci. 25, 7289-7298 (2005).
10. K. Tatsumi, A. Isonishi, M. Yamasaki, Y. Kawabe, S. Morita-Takemura, K. Nakahara, Y. Terada, T. Shinjo, H. Okuda, T. Tanaka, A. Wanaka, Olig2-Lineage astrocytes: A distinct subtype of astrocytes that differs from GFAP astrocytes. Front. Neuroanat. 12, 8 (2018), doi: 10.3389/FNANA.2018.00008/FULL.
11. Z. Liu, X. Hu, J. Cai, B. Liu, X. Peng, M. Wegner, M. Qiu, Induction of oligodendrocyte differentiation by Olig2 and Sox10: Evidence for reciprocal interactions and dosage-dependent mechanisms. Dev. Biol. 302, 683-693 (2007).
12. C. D. Pozniak, A. J. Langseth, G. J. P. Dijkgraaf, Y. Choe, Z. Werb, S. J. Pleasure, Sox10 directs neural stem cells toward the oligodendrocyte lineage by decreasing Suppressor of Fused expression. Proc. Natl. Acad. Sci. U.S.A 107, 21795-21800 (2010).
13. N. Takada, S. Kucenas, B. Appel, Sox10 is necessary for oligodendrocyte survival following axon wrapping. Glia. 58, 996-1006 (2010).
14. R. Awatramani, S. S. Scherer, J. B. Grinspan, E. Collarini, R. Skoff, D. O'Hagan, J. Garbern, J. Kamholz, Evidence that the homeodomain protein Gtx is involved in the regulation of oligodendrocyte myelination. J. Neurosci. 17, 6657-6668 (1997).
15. J. Cai, Q. Zhu, K. Zheng, H. Li, Y. Qi, Q. Cao, M. Qiu, Co-localization of Nkx6.2 and Nkx2.2 homeodomain proteins in differentiated myelinating oligodendrocytes. Glia. 58, 458-468 (2010).
16. S. W. Levison, J. E. Goldman, Both oligodendrocytes and astrocytes develop from progenitors in the subventricular zone of postnatal rat forebrain. Neuron. 10, 201-212 (1993).
17. A. Kriegstein, A. Alvarez-Buylla, The Glial Nature of Embryonic and Adult Neural Stem Cells. Annu. Rev. Neurosci. 32, 149-184 (2009).
18. G. Masserdotti, S. Gascón, M. Götz, Direct neuronal reprogramming: Learning from and for development. Dev. 143 (2016), pp. 2494-2510.
19. E. Hörmanseder, Epigenetic memory in reprogramming. Curr. Opin. Genet. Dev. 70, 24-31 (2021).
20. A. M. Khanghahi, L. Satarian, W. Deng, H. Baharvand, M. Javan, In vivo conversion of astrocytes into oligodendrocyte lineage cells with transcription factor Sox10; Promise for myelin repair in multiple sclerosis. PLOS One. 13 (2018), doi: 10.1371/journal.pone.0203785.
21. X. Hu, S. Qin, X. Huang, Y. Yuan, Z. Tan, Y. Gu, X. Cheng, D. Wang, X. F. Lian, C. He, Z. Su, Region-Restrict Astrocytes Exhibit Heterogeneous Susceptibility to Neuronal Reprogramming. Stem Cell Reports. 12, 290-304 (2019).
22. N. Behrangi, P. Lorenz, M. Kipp, Oligodendrocyte Lineage Marker Expression in eGFP-GFAP Transgenic Mice. J. Mol. Neurosci. (2020), doi: 10.1007/s12031-020-01771-w.
23. R. P. Gorter, W. Baron, Matrix metalloproteinases shape the oligodendrocyte (niche) during development and upon demyelination. Neurosci. Lett. 729, 134980 (2020).
24. E. Ercan, J. M. Han, A. Di Nardo, K. Winden, M. J. Han, L. Hoyo, A. Saffari, A. Leask, D. H. Geschwind, M. Sahin, Neuronal CTGF/CCN2 negatively regulates myelination in a mouse model of tuberous sclerosis complex. J. Exp. Med. 214, 681-697 (2017).
25. S. R. Clarke, A. K. Shetty, J. L. Bradley, D. A. Turner, Reactive astrocytes express the embryonic intermediate neurofilament nestin. Neuroreport. 5, 1885-1888 (1994).
26. S. A. Liddelow, K. A. Guttenplan, L. E. Clarke, F. C. Bennett, C. J. Bohlen, L. Schirmer, M. L. Bennett, A. E. Münch, W. S. Chung, T. C. Peterson, D. K. Wilton, A. Frouin, B. A. Napier, N. Panicker, M. Kumar, M. S. Buckwalter, D. H. Rowitch, V. L. Dawson, T. M. Dawson, B. Stevens, B. A. Barres, Neurotoxic reactive astrocytes are induced by activated microglia. Nature. 541, 481-487 (2017).
27. J. D. Rothstein, L. Martin, A. I. Levey, M. Dykes-Hoberg, L. Jin, D. Wu, N. Nash, R. W. Kuncl, Localization of neuronal and glial glutamate transporters. Neuron. 13, 713-725 (1994).
28. K. P. Lehre, L. M. Levy, O. P. Ottersen, J. Storm-Mathisen, N. C. Danbolt, Differential expression of two glial glutamate transporters in the rat brain: quantitative and immunocytochemical observations. J. Neurosci. 15, 1835-1853 (1995).
29. X. Y. Xiong, Y. Tang, Q. W. Yang, Metabolic changes favor the activity and heterogeneity of reactive astrocytes. Trends Endocrinol. Metab. 33, 390-400 (2022).
30. R. A. Sobel, A. S. Ahmed, White Matter Extracellular Matrix Chondroitin Sulfate/Dermatan Sulfate Proteoglycans in Multiple Sclerosis. J. Neuropathol. Exp. Neurol. 60, 1198-1207 (2001).
31. J. D. Figueroa, M. Serrano-Illan, J. Licero, K. Cordero, J. D. Miranda, M. De Leon, Fatty Acid Binding Protein 5 Modulates Docosahexaenoic Acid-Induced Recovery in Rats Undergoing Spinal Cord Injury. J. Neurotrauma. 33, 1436-1449 (2016).
32. A. Schulte, L. Bieniussa, R. Gupta, S. Samtleben, T. Bischler, K. Doering, P. Sodmann, H. Rittner, R. Blum, Homeostatic calcium fluxes, ER calcium release, SOCE, and calcium oscillations in cultured astrocytes are interlinked by a small calcium toolkit. Cell Calcium. 101, 102515 (2022).
33. S. Marques, A. Zeisel, S. Codeluppi, D. Van Bruggen, A. M. Falcão, L. Xiao, H. Li, M. Häring, H. Hochgerner, R. A. Romanov, D. Gyllborg, A. B. Muñoz-Manchado, G. La Manno, P. Lönnerberg, E. M. Floriddia, F. Rezayee, P. Ernfors, E. Arenas, J. Hjerling-Leffler, T. Harkany, W. D. Richardson, S. Linnarsson, G. Castelo-Branco, Oligodendrocyte heterogeneity in the mouse juvenile and adult central nervous system. Science (80-.). 352, 1326-1329 (2016).
34. J. C. Dugas, Y. C. Tai, T. P. Speed, J. Ngai, B. A. Barres, Functional genomic analysis of oligodendrocyte differentiation. J. Neurosci. 26, 10967-10983 (2006).
35. M. Lattke, R. Goldstone, J. K. Ellis, S. Boeing, J. Jurado-Arjona, N. Marichal, J. I. MacRae, B. Berninger, F. Guillemot, Extensive transcriptional and chromatin changes underlie astrocyte maturation in vivo and in culture. Nat. Commun. 2021 121. 12, 1-18 (2021).
36. N. Yang, J. B. Zuchero, H. Ahlenius, S. Marro, Y. H. Ng, T. Vierbuchen, J. S. Hawkins, R. Geissler, B. A. Barres, M. Wernig, Generation of oligodendroglial cells by direct lineage conversion. Nat. Biotechnol. 31, 434-439 (2013).
37. C. N. Svendsen, M. V. Sofroniew, Lineage tracing: The gold standard to claim direct reprogramming in vivo. Mol. Ther. 30, 988-989 (2022).
38. K. D. Mccarthy, J. De Vellis, Preparation of separate astroglial and oligodendroglial cell cultures from rat cerebral tissue. J. Cell Biol. 85, 890-902 (1980).
39. N. L. Bray, H. Pimentel, P. Melsted, L. Pachter, Near-optimal probabilistic RNA-seq quantification. Nat. Biotechnol. 2016 345. 34, 525-527 (2016).
40. P. Melsted, A. S. Booeshaghi, L. Liu, F. Gao, L. Lu, K. H. (Joseph) Min, E. da Veiga Beltrame, K. E. Hjörleifsson, J. Gehring, L. Pachter, Modular, efficient and constant-memory single-cell RNA-seq preprocessing. Nat. Biotechnol. 39, 813-818 (2021).
41. Y. Hao, S. Hao, E. Andersen-Nissen, W. M. Mauck, S. Zheng, A. Butler, M. J. Lee, A. J. Wilk, C. Darby, M. Zager, P. Hoffman, M. Stoeckius, E. Papalexi, E. P. Mimitou, J. Jain, A. Srivastava, T. Stuart, L. M. Fleming, B. Yeung, A. J. Rogers, J. M. McElrath, C. A. Blish, R. Gottardo, P. Smibert, R. Satija, Integrated analysis of multimodal single-cell data. Cell. 184, 3573-3587.e29 (2021).
42. Z. Gu, R. Eils, M. Schlesner, Complex heatmaps reveal patterns and correlations in multidimensional genomic data. Bioinformatics. 32, 2847-2849 (2016).
43. K. Street, D. Risso, R. B. Fletcher, D. Das, J. Ngai, N. Yosef, E. Purdom, S. Dudoit, Slingshot: cell lineage and pseudotime inference for single-cell transcriptomics. BMC Genomics. 19 (2018), doi: 10.1186/S12864-018-4772-0.
44. M. Kuhn, H. Wickham, Tidymodels: a collection of packages for modeling and machine learning using tidyverse principles (2020), (available at https://www.tidymodels.org/).
45. M. Kuhn, D. Vaughan, Parsnip: A Common API to Modeling and Analysis Functions•parsnip (2022), (available at https://parsnip.tidymodels.org/).
46. H. Wickham, ggplot2: Elegant Graphics for Data Analysis (Springer-Verlag, New York, 2016), vol. 2.
47. RStudio Team, RStudio: Integrated Development for R. (2020), (available at http://www.rstudio.com/.).
48. GraphPad Software, GraphPad Prism (2022).
49. Bajohr, J. & Faiz, M. Direct Lineage Reprogramming in the CNS. in Cell Biology and Translational Medicine, Volume 6: Stem Cells: Their Heterogeneity, Niche and Regenerative Potential (ed. Turksen, K.) 31-48 (Springer International Publishing, Cham, 2020). doi: 10.1007/5584_2019_374.
50. Heinrich, C. et al. Directing astroglia from the cerebral cortex into subtype specific functional neurons. PLOS Biol 8, e1000373 (2010).
51. Liu, Y. et al. Ascl1 Converts Dorsal Midbrain Astrocytes into Functional Neurons In Vivo. J Neurosci 35, 9336-9355 (2015).
52. Bussmann, L. H. et al. A Robust and Highly Efficient Immune Cell Reprogramming System. Cell Stem Cell 5, 554-566 (2009).
53. Xie, H., Ye, M., Feng, R. & Graf, T. Stepwise reprogramming of B cells into macrophages. Cell 117, 663-676 (2004).
54. Wang, H. et al. Region-specific distribution of Olig2-expressing astrocytes in adult mouse brain and spinal cord. Molecular Brain 14, 36 (2021).
55. Buffo, A. et al. Expression pattern of the transcription factor Olig2 in response to brain injuries: Implications for neuronal repair. Proc Natl Acad Sci USA 102, 18183-18188 (2005).
56. Stolt, C. C. et al. Terminal differentiation of myelin-forming oligodendrocytes depends on the transcription factor Sox10. Genes Dev 16, 165-170 (2002).
57. Wang, L.-L. et al. Revisiting astrocyte to neuron conversion with lineage tracing in vivo. Cell 184, 5465-5481.e16 (2021).
58. Marsh, S. scCustomize: Custom Visualizations & Functions for Streamlined Analyses of Single Cell Sequencing. (2021).
59. Trapnell, C. et al. The dynamics and regulators of cell fate decisions are revealed by pseudotemporal ordering of single cells. Nat Biotechnol 32, 381-386 (2014).
60. Qiu, X. et al. Reversed graph embedding resolves complex single-cell trajectories. Nat Methods 14, 979-982 (2017).
61. Cao, J. et al. The single cell transcriptional landscape of mammalian organogenesis. Nature 566, 496-502 (2019).
62. Traag, V. A., Waltman, L. & van Eck, N. J. From Louvain to Leiden: guaranteeing well-connected communities. Sci Rep 9, 5233 (2019).
63. Levine, J. H. et al. Data-driven phenotypic dissection of AML reveals progenitor-like cells that correlate with prognosis. Cell 162, 184-197 (2015).
64. Kamimoto, K. et al. Dissecting cell identity via network inference and in silico gene perturbation. Nature 614, 742-751 (2023).
65. Sadria, M., Layton, A., Goyal, S. & Bader, G. D. Fatecode: Cell fate regulator prediction using classification autoencoder perturbation. 2022.12.16.520772 Preprint at https://doi.org/10.1101/2022.12.16.520772 (2022).
66. Fard, M. K. et al. BCAS1 expression defines a population of early myelinating oligodendrocytes in multiple sclerosis lesions. Sci Transl Med 9, eaam7816 (2017).
67. Vourc'h, P. & Andres, C. Oligodendrocyte myelin glycoprotein (OMgp): evolution, structure and function. Brain Res Brain Res Rev 45, 115-124 (2004).
68. Mikol, D. D. & Stefansson, K. A phosphatidylinositol-linked peanut agglutinin-binding glycoprotein in central nervous system myelin and on oligodendrocytes. J Cell Biol 106, 1273-1279 (1988).
69. Bi, F. et al. Reactive astrocytes secrete Icn2 to promote neuron death. Proceedings of the National Academy of Sciences 110, 4069-4074 (2013).
70. Lee, W.-H., Wang, G.-M., Seaman, L. B. & Vannucci, S. J. Coordinate IGF-I and IGFBP5 Gene Expression in Perinatal Rat Brain after Hypoxia-Ischemia. J Cereb Blood Flow Metab 16, 227-236 (1996).
71. Morita, K., Sasaki, H., Fujimoto, K., Furuse, M. & Tsukita, S. Claudin-11/OSP-based Tight Junctions of Myelin Sheaths in Brain and Sertoli Cells in Testis. J Cell Biol 145, 579-588 (1999).
72. Kister, A. & Kister, I. Overview of myelin, major myelin lipids, and myelin-associated proteins. Front. Chem. 10, (2023).
73. Hoffmann, S. A. et al. Stem cell factor Sox2 and its close relative Sox3 have differentiation functions in oligodendrocytes. Development 141, 39-50 (2014).
74. Dugas, J. C., Ibrahim, A. & Barres, B. A. The T3-induced gene KLF9 regulates oligodendrocyte differentiation and myelin regeneration. Mol Cell Neurosci 50, 45-57 (2012).
75. Sock, E. & Wegner, M. Using the lineage determinants Olig2 and Sox10 to explore transcriptional regulation of oligodendrocyte development. Dev Neurobiol 81, 892-901 (2021).
76. Lee, Y., Messing, A., Su, M. & Brenner, M. GFAP promoter elements required for region-specific and astrocyte-specific expression. Glia 56, 481-493 (2008).
77. Chen, Y.-C. et al. A NeuroD1 AAV-Based Gene Therapy for Functional Brain Repair after Ischemic Injury through In Vivo Astrocyte-to-Neuron Conversion. Molecular Therapy 28, 217-234 (2020).
78. Qian, H. et al. Reversing a model of Parkinson's disease with in situ converted nigral neurons. Nature 582, 550-556 (2020).
79. Janeckova, L. et al. Astrocyte-like subpopulation of NG2 glia in the adult mouse cortex exhibits characteristics of neural progenitor cells. Glia 72, 245-273 (2024).
80. Kirdajova, D. et al. Transient astrocyte-like NG2 glia subpopulation emerges solely following permanent brain ischemia. Glia 69, 2658-2681 (2021).
81. Rusnakova, V. et al. Heterogeneity of Astrocytes: From Development to Injury-Single Cell Gene Expression. PLOS ONE 8, e69734 (2013).
82. Barres, B. A., Jacobson, M. D., Schmid, R., Sendtner, M. & Raff, M. C. Does oligodendrocyte survival depend on axons? Current Biology 3, 489-497 (1993).
83. Macklin, W. b., Weill, C. I. & Deininger, P. I. Expression of myelin proteolipid and basic protein mRNAS in cultured cells. Journal of Neuroscience Research 16, 203-217 (1986).
84. Rosenberg, S. S., Kelland, E. E., Tokar, E., De La Torre, A. R. & Chan, J. R. The geometric and spatial constraints of the microenvironment induce oligodendrocyte differentiation. Proc Natl Acad Sci USA 105, 14662-14667 (2008).
85. Lee, S. et al. A culture system to study oligodendrocyte myelination-processes using engineered nanofibers. Nat Methods 9, 917-922 (2012).
86. Bush, T. G. et al. Leukocyte infiltration, neuronal degeneration, and neurite outgrowth after ablation of scar-forming, reactive astrocytes in adult transgenic mice. Neuron 23, 297-308 (1999).
87. Davis, N. et al. Pharmacological ablation of astrocytes reduces AB degradation and synaptic connectivity in an ex vivo model of Alzheimer's disease. J Neuroinflammation 18, 73 (2021).
88. Gascón, S., Masserdotti, G., Russo, G. L. & Götz, M. Direct Neuronal Reprogramming: Achievements, Hurdles, and New Roads to Success. Cell Stem Cell 21, 18-34 (2017).

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

We claim:

1. An in vivo method for production of induced oligodendrocyte lineage cells (iOLCs) from donor astrocytes comprising contacting the donor astrocytes with a polynucleotide comprising a nucleic acid encoding a single transcription factor (TF), wherein the single TF is Olig2, Sox10, or Nkx6.2, wherein the nucleic acid encoding the single TF is operably linked to an astrocyte-specific promoter sequence.

2. The method of claim 1, wherein the donor astrocytes are GFAP+cortical astrocytes.

3. The method of claim 1, wherein the polynucleotide is within a vector, which is a viral vector, such as an adenoviral vector, an adeno-associated viral vector or a lentiviral vector, or a non-viral vector.

4. The method of claim 1, wherein the polynucleotide is packaged in a nanoparticle, such as a lipid nanoparticle.

5. The method of claim 1, wherein the single TF is Olig2 and the iOLCs are induced oligodendrocyte progenitor cells.

6. The method of claim 1, wherein the single TF is Sox10 and the iOLCs are myelinating olidodendrocytes and/or pre-myelinating oligodendrocyte precursor cells.

7. The method of claim 1, wherein the single TF is is Nkx6.2 and the iOLCs are myelinating olidodendrocytes.

8. A vector comprising a nucleic acid encoding a single transcription factor (TF), wherein the single TF is Olig2, Sox10, or Nkx6.2, and wherein the nucleic acid encoding the single TF is operably linked to an astrocyte-specific promoter sequence.

9. The vector of claim 8, wherein the vector is a viral vector, such as an adenoviral vector, an adeno-associated viral vector or a lentiviral vector.

10. The vector of claim 8, wherein the astrocyte-specific promoter sequence is a glial fibrillary acidic protein (GFAP) promoter.

11. A viral particle comprising the vector of claim 8.

12. A host cell comprising the vector of claim 8.

13. A pharmaceutical composition comprising a nucleic acid encoding a single transcription factor (TF), wherein the single TF is Olig2, Sox10, or Nkx6.2, and wherein the nucleic acid encoding the single TF is operably linked to an astrocyte-specific promoter sequence and a pharmaceutically acceptable carrier or excipient.

14. The pharmaceutical composition of claim 13, wherein the nucleic acid is in a viral vector, a non-viral vector or a viral particle.

15. A method for prevention or treatment of a disease or disorder that affects astrocyte and/or oligodendrocyte lineage cells in a subject, said method comprising administering to the subject a nucleic acid for expression of a single transcription factor (TF), wherein the single TF is Olig2, Sox10, or Nkx6.2, in astrocytes within the subject.

16. The method according to claim 15, wherein the disease or disorder is a myelin-related disease or disorder.

17. The method of claim 15, wherein the nucleic acid is in a vector and wherein the nucleic acid encoding the single TF is operably linked to an astrocyte-specific promoter sequence.

18. The method of claim 15, wherein the nucleic acid is in a pharmaceutical composition and the method comprises administering the composition to the subject via direct intracranial administration, intracranial injection, intracerebroventricular injection, intracisternal injection, intrathecal injection, intravenous injection, intraperitoneal injection, intraarterial, delivery via nanoparticles, and/or administration using focused ultrasound.


SEQUENCE LISTING (7 sequences)

SEQ ID NO: 1 - DNA, 969 bp, Mus musculus
  source 1..969: mol_type = other DNA
atggactcggacgccagcctggtgtctagtcgcccatcgtccccagaacccgatgatctttttctgcccgcccggagcaaggggggaagcagcagcggcttcacaggagggactgtgtcctcgtccacgccgagcgactgcccgccagagctgagctccgagctacgaggggccatgggcgcgtcaggcgcgcatcccggggacaaactgggcggcggtggcttcaagtcatcttcctccagcacctcctcgtccacgtcctcggcggccacgtcttccaccaagaaagacaagaagcagatgactgagcccgagctgcagcagctgcgcctgaagatcaacagccgcgaacgcaagcgcatgcacgacctcaacatcgccatggacggcctgcgggaggtcatgccttacgcgcacgggccgtcggtgcgcaagctctccaagatcgccacgctgctgctggcgcgaaactacatcctcatgctcaccaactcgctggaggagatgaagcgactggtgagcgagatctacgggggtcaccacgccggcttccacccatcggcctgtggcgggctggcacactcggcgcctctacccaccgccacggcgcaccccgcggccgccgcgcacgccgcgcaccacccggccgtgcaccaccccatcctccctcccgcagccgccgccgccgccgcagccgccgccgccgcagcggtgtccagcgcctctctaccgggctccgggctgtcctcggtcggatccatccggcctccgcacggcctgctcaagtcaccgtcggcagccgcagccgcccctctcggaggcgggggtggaggcagcggtggcagcggtggcttccagcactggggcggcatgccgtgcccctgcagcatgtgccaggtgccgccgccgcaccaccacgtgtcggctatgggcgccggcaccttgccgcgcctcacctccgacgccaag

SEQ ID NO: 2 - DNA, 1398 bp, Mus musculus
  source 1..1398: mol_type = other DNA
atggccgaggaacaagacctatcagaggtggagctgagccctgtgggctcggaggaaccccgctgcctgtccccaggcagcgcgccgtcgctgggacccgacggcggcggcggtggctcgggcttgcgagccagcccggggcccggtgaactgggcaaggtcaagaaggaacagcaggacggcgaggcggacgatgacaagttccccgtgtgcatccgcgaggcggtcagccaggtgctcagcggctacgactggacgctggtgcccatgcccgtgcgcgtcaacggtgccagcaagagcaagccgcacgtcaagaggcccatgaacgccttcatggtgtgggcacaggcggcacgcagaaagctagccgaccagtaccctcacctccacaatgctgagctcagcaagacactaggcaagctctggaggttgctgaacgaaagtgacaagcgccccttcattgaggaggctgagcggctccggatgcagcacaaaaaggaccatccggactacaagtaccaacctcggcggcggaagaacgggaaggcagcccagggggaggcagaatgcccaggcggggaagccgagcaaggaggggctgctgctattcaggctcactacaagagtgcccacctggaccaccggcacccagaagaaggctcccccatgtcagatgggaacccagagcacccctcaggccagagccatggccccccaacccctccaaccaccccaaagacagagctgcagtccggcaaggcagaccccaaaagggatgggcgctccttgggggagggcgggaagccccacatcgacttcggcaacgtggacatcggggagatcagccacgaggtaatgtccaacatggaaacctttgatgtgactgagctggaccaatacctgccacccaacgggcacccaggccatgtgggtagctactcggcagctggctatgggctgggcagtgccctggctgtggccagtggacactctgcctggatctccaagccaccaggtgtggctctgcccacggtctcgccccctggtgtggatgccaaagcccaggtgaagacagagaccacaggcccccagggacccccacactacaccgaccagccgtccacttcccagatcgcctacacctccctcagtctgccccactacggctccgccttcccctccatctcacgaccccagtttgactattctgaccatcagccctcaggaccctattatggccatgcaggccaggcctctggcctctattcagccttttcttacatggggccctcccagcggcccctctacactgccatctctgaccccagcccctcggggccccaatcccacagtcccacacactgggagcagccagtatatacgactctatcccgacct

SEQ ID NO: 3 - DNA, 831 bp, Mus musculus
  source 1..831: mol_type = other DNA
atggacgctaaccgcccgggtgcgtttgtgctgagcagcgcgcctttagccgcgctgcacaacatggctgagatgaagacgtcgctgttcccctacgcgctgcagggcccggcgggcttcaagacacccgccctaggcagccttggcgcgcagttgcctctaggcactccgcacggcatcagcgacatcctgggacggccggtgggcgcagcgggtggcggcctcctaggaagtctgccccgtctcaacgggctcgcctcgtctgcaggtgtctacttcgggcccgcagccgccgtggctcggggctaccccaagccgctggcggaactgcctgggcgcccgcccatcttctggcctggggtggtgcagggctctccctggagggacccgcgactggccggctccgcccaagccggcggggtcctggataaggatggcaagaagaaacactcgcggccgactttctccggccagcagatcttcgcgctggagaagactttcgagcagaccaagtatttggcaggcccagagcgcgcgcggcttgcctactctctgggcatgaccgagagccaagtgaaggtgtggttccagaatcggcggaccaagtggcgcaagcggcacgcggcagagatggcgtcggctaaaaagaagcaagactcggatgccgagaagctgaaggtgggtggctcagacgcggaggacgatgacgaatacaaccggcccctggaccccaactccgatgacgagaagatcacgcggcttctcaaaaagcacaaaccctcgaacttggcgctcgttagcccgtgtggtggcagcgcgggggacgccttg

SEQ ID NO: 4 - DNA, 10455 bp, synthetic construct
  source 1..10455: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttggagctcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgcgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggcaagtttgtacaaaaaagcaggctgccaccatggactcggacgccagcctggtgtctagtcgcccatcgtccccagaacccgatgatctttttctgcccgcccggagcaaggggggaagcagcagcggcttcacaggagggactgtgtcctcgtccacgccgagcgactgcccgccagagctgagctccgagctacgaggggccatgggcgcgtcaggcgcgcatcccggggacaaactgggcggcggtggcttcaagtcatcttcctccagcacctcctcgtccacgtcctcggcggccacgtcttccaccaagaaagacaagaagcagatgactgagcccgagctgcagcagctgcgcctgaagatcaacagccgcgaacgcaagcgcatgcacgacctcaacatcgccatggacggcctgcgggaggtcatgccttacgcgcacgggccgtcggtgcgcaagctctccaagatcgccacgctgctgctggcgcgaaactacatcctcatgctcaccaactcgctggaggagatgaagcgactggtgagcgagatctacgggggtcaccacgccggcttccacccatcggcctgtggcgggctggcacactcggcgcctctacccaccgccacggcgcaccccgcggccgccgcgcacgccgcgcaccacccggccgtgcaccaccccatcctccctcccgcagccgccgccgccgccgcagccgccgccgccgcagcggtgtccagcgcctctctaccgggctccgggctgtcctcggtcggatccatccggcctccgcacggcctgctcaagtcaccgtcggcagccgcagccgcccctctcggaggcgggggtggaggcagcggtggcagcggtggcttccagcactggggcggcatgccgtgcccctgcagcatgtgccaggtgccgccgccgcaccaccacgtgtcggctatgggcgccggcaccttgccgcgcctcacctccgacgccaagggaagcggagccacgaacttctctctgttaaagcaagcaggagatgttgaagaaaaccccgggcctatgggcccaaagaagaagagaaaggtttcgaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 5 - DNA, 10884 bp, synthetic construct
  source 1..10884: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttggagctcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgcgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggcaagtttgtacaaaaaagcaggctgccaccatggccgaggaacaagacctatcagaggtggagctgagccctgtgggctcggaggaaccccgctgcctgtccccaggcagcgcgccgtcgctgggacccgacggcggcggcggtggctcgggcttgcgagccagcccggggcccggtgaactgggcaaggtcaagaaggaacagcaggacggcgaggcggacgatgacaagttccccgtgtgcatccgcgaggcggtcagccaggtgctcagcggctacgactggacgctggtgcccatgcccgtgcgcgtcaacggtgccagcaagagcaagccgcacgtcaagaggcccatgaacgccttcatggtgtgggcacaggcggcacgcagaaagctagccgaccagtaccctcacctccacaatgctgagctcagcaagacactaggcaagctctggaggttgctgaacgaaagtgacaagcgccccttcattgaggaggctgagcggctccggatgcagcacaaaaaggaccatccggactacaagtaccaacctcggcggcggaagaacgggaaggcagcccagggggaggcagaatgcccaggcggggaagccgagcaaggaggggctgctgctattcaggctcactacaagagtgcccacctggaccaccggcacccagaagaaggctcccccatgtcagatgggaacccagagcacccctcaggccagagccatggccccccaacccctccaaccaccccaaagacagagctgcagtccggcaaggcagaccccaaaagggatgggcgctccttgggggagggcgggaagccccacatcgacttcggcaacgtggacatcggggagatcagccacgaggtaatgtccaacatggaaacctttgatgtgactgagctggaccaatacctgccacccaacgggcacccaggccatgtgggtagctactcggcagctggctatgggctgggcagtgccctggctgtggccagtggacactctgcctggatctccaagccaccaggtgtggctctgcccacggtctcgccccctggtgtggatgccaaagcccaggtgaagacagagaccacaggcccccagggacccccacactacaccgaccagccgtccacttcccagatcgcctacacctccctcagtctgccccactacggctccgccttcccctccatctcacgaccccagtttgactattctgaccatcagccctcaggaccctattatggccatgcaggccaggcctctggcctctattcagccttttcttacatggggccctcccagcggcccctctacactgccatctctgaccccagcccctcggggccccaatcccacagtcccacacactgggagcagccagtatatacgactctatcccgacctggaagcggagccacgaacttctctctgttaaagcaagcaggagatgttgaagaaaaccccgggcctatgggcccaaagaagaagagaaaggtttcgaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 6 - DNA, 10317 bp, synthetic construct
  source 1..10317: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttggagctcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgcgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggcaagtttgtacaaaaaagcaggctgccaccatggacgctaaccgcccgggtgcgtttgtgctgagcagcgcgcctttagccgcgctgcacaacatggctgagatgaagacgtcgctgttcccctacgcgctgcagggcccggcgggcttcaagacacccgccctaggcagccttggcgcgcagttgcctctaggcactccgcacggcatcagcgacatcctgggacggccggtgggcgcagcgggtggcggcctcctaggaagtctgccccgtctcaacgggctcgcctcgtctgcaggtgtctacttcgggcccgcagccgccgtggctcggggctaccccaagccgctggcggaactgcctgggcgcccgcccatcttctggcctggggtggtgcagggctctccctggagggacccgcgactggccggctccgcccaagccggcggggtcctggataaggatggcaagaagaaacactcgcggccgactttctccggccagcagatcttcgcgctggagaagactttcgagcagaccaagtatttggcaggcccagagcgcgcgcggcttgcctactctctgggcatgaccgagagccaagtgaaggtgtggttccagaatcggcggaccaagtggcgcaagcggcacgcggcagagatggcgtcggctaaaaagaagcaagactcggatgccgagaagctgaaggtgggtggctcagacgcggaggacgatgacgaatacaaccggcccctggaccccaactccgatgacgagaagatcacgcggcttctcaaaaagcacaaaccctcgaacttggcgctcgttagcccgtgtggtggcagcgcgggggacgccttgggaagcggagccacgaacttctctctgttaaagcaagcaggagatgttgaagaaaaccccgggcctatgggcccaaagaagaagagaaaggtttcgaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 7 - DNA, 10185 bp, synthetic construct
  source 1..10185: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttggagctcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgcgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggcaagtttgtacaaaaaagcaggctgccaccatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatggaagcggagccacgaacttctctctgttaaagcaagcaggagatgttgaagaaaaccccgggcctatgggcccaaagaagaagagaaaggtttcgaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt